(19) United States
(12) Patent Application Publication
(10) Pub. No.: US 2026/0206768 A1
(43) Pub. Date: Jul. 23, 2026

(54) COMBINATION OF VARIOUS NITROGEN FIXING BACTERIA WITH VARIOUS BIOLOGICAL PRODUCTS TO ACHIEVE SYNERGISTIC EFFECTS

(71) Applicant: Pivot Bio, Inc., Minnetonka, MN (US)

(72) Inventors: Chad KIMMELSHUE, Richmond Heights, MO (US); Trevor JONES, West Des Moines, IA (US); Keith O'BRYAN, Omaha, NE (US); Daniel POSTON, Owens Cross Roads, AL (US); John HENKHAUS, St. Louis, MO (US); Ernest SANDERS, Wright City, MO (US); Alvin TAMSIR, Alamo, CA (US)

(73) Assignee: Pivot Bio, Inc., Minnetonka, MN (US)

(21) Appl. No.: 19/140,733

(22) PCT Filed: Dec. 11, 2023
(86) PCT No.: PCT/US2023/083413

§ 371 Date: Jun. 18, 2025

Related U.S. Application Data
(60) Provisional application No. 63/435,193, filed on Dec. 23, 2022.

(51) Int. Cl. A01N 63/20; A01P 1/00; A01P 21/00; C05F 11/08; C12N 1/20; C12N 9/00; C12R 1/22

(52) U.S. Cl. CPC A01N 63/20; A01P 1/00; A01P 21/00; C05F 11/08; C12N 1/20; C12N 9/00; C12R 2001/22

(57) ABSTRACT

The present disclosure provides compositions, kits, and systems comprising engineered bacteria and agricultural biologicals, and methods of use thereof to treat a plant and improve one or more plant beneficial traits, such as plant yield, nutrient uptake, and resistance to an abiotic or biotic stress. Use of the compositions, kits, and systems disclosed herein may further 5 decrease usage of fertilizers. The disclosure further teaches methods of using engineered bacteria with agricultural biologicals to treat a plant.

### CROSS-REFERENCE TO RELATED APPLICATIONS

This application claims the benefit of Provisional Patent Application No. 63/435,193, filed on Dec. 23, 2022, which is hereby incorporated by reference in its entirety for all purposes.

### FIELD

The present disclosure relates to compositions, methods, kits, and systems comprising microbes and biologicals to treat a plant. The compositions, methods, kits, and systems disclosed herein may be used, for example, to increase nitrogen fixation, improve plant yield, nutrient uptake, resistance to abiotic and biotic stressors, and may have other beneficial effects on agricultural crops.

### BACKGROUND

Application of plant beneficial microbes, such as nitrogen-fixing bacteria, to crops can increase agricultural yield, while potentially decreasing the use of fertilizers. These beneficial microbes can be cultured and transplanted to the soil near the root structure of the plant, or alternatively may be formulated in a seed coating. Agricultural biologicals, such as biostimulants, biopesticides, and biofertilizers can also increase yield and reduce the use of fertilizer, and further can increase resistance to abiotic and biotic stressors such as deficient or excessive water and high salinity.

In order to meet the world's growing food supply needs—while also balancing resource utilization and providing minimal impacts upon environmental systems—a better approach to growing crops is urgently needed. Thus, there is a need in the art for compositions and methods that utilize the synergistic effects of nitrogen fixing bacteria and agricultural biologicals.

### SUMMARY

The disclosure provides a composition comprising: a plurality of engineered bacteria having at least one genetic variation introduced into at least one gene, or non-coding polynucleotide, of the nitrogen fixation or assimilation genetic regulatory network; and b) an agricultural biological. In some embodiments, the engineered bacteria are non-intergeneric remodeled bacteria. In some embodiments, the engineered bacteria are transgenic bacteria. The disclosure further teaches methods of applying the composition to a plant or to an area in which a plant will be grown or is growing.

The disclosure further provides a method of treating a plant, comprising: applying a plurality of engineered bacteria having at least one genetic variation introduced into at least one gene, or non-coding polynucleotide, of the nitrogen fixation or assimilation genetic regulatory network to a plant or to an area in which a plant will be grown or is growing; and applying an agricultural biological to the plant or to an area in which a plant will be grown or is growing. In some embodiments, the engineered bacteria are non-intergeneric remodeled bacteria. In some embodiments, the engineered bacteria are transgenic bacteria. In some embodiments, the plurality of engineered bacteria and the agricultural biological are applied simultaneously. In some embodiments, the plurality of engineered bacteria and the agricultural biological are applied separately.

The disclosure further provides a kit comprising: a) a plurality of engineered bacteria having at least one genetic variation introduced into at least one gene, or non-coding polynucleotide, of the nitrogen fixation or assimilation genetic regulatory network; and b) an agricultural biological.

The disclosure further provides a farm administration system, comprising: a) a plurality of engineered bacteria having at least one genetic variation introduced into at least one gene, or non-coding polynucleotide, of the nitrogen fixation or assimilation genetic regulatory network; b) an agricultural biological; and c) instructions for using a) and b) to treat a plant.

### DETAILED DESCRIPTION

### Definitions

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Throughout this application, the term “about” is used to indicate that a value includes the inherent variation of error for the device, or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

As used herein “plant” refers to any part of the plant, including plant cells and plant parts during any aspect of the growing cycle, including seeds, seedlings, plants, leaves, roots, root hairs, rhizomes, stems, seed, ovules, pollen, flowers, fruit, cuttings, tubers, bulbs, etc.

“Plant productivity” refers generally to any aspect of growth or development of a plant that is a reason for which the plant is grown. For food crops, such as grains or vegetables, “plant productivity” can refer to the yield of grain or fruit harvested from a particular crop. As used herein, improved plant productivity refers broadly to improvements in yield of grain, fruit, flowers, or other plant parts harvested for various purposes, improvements in growth of plant parts, including stems, leaves and roots, promotion of plant growth, maintenance of high chlorophyll content in leaves, increasing fruit or seed numbers, increasing fruit or seed unit weight, and similar improvements of the growth and development of plants. In some embodiments, plant productivity is determined by comparing the productivity (e.g., yield) of a treated plant or plant part (e.g., via a seed coating, in-furrow application, or foliar spray as described herein), vs. an untreated plant or plant part.

Microbes in and around food crops can influence the traits of those crops. Plant traits that may be influenced by microbes include: yield (e.g., grain production, biomass generation, fruit development, flower set); nutrition (e.g., nitrogen, phosphorus, potassium, iron, micronutrient acquisition); abiotic stress management (e.g., drought tolerance, salt tolerance, heat tolerance); and biotic stress management (e.g., pest, weeds, insects, fungi, and bacteria). Strategies for altering crop traits include: increasing key metabolite concentrations; changing temporal dynamics of microbe influence on key metabolites; linking microbial metabolite production/degradation to new environmental cues; reducing negative metabolites; and improving the balance of metabolites or underlying proteins.

As used herein, “in planta” refers to in the plant, on the plant, or intimately associated with the plant, depending upon context of usage (e.g. endophytic, epiphytic, or rhizospheric associations). As used herein, the term “plant” can include plant parts, tissue, leaves, roots, root hairs, rhizomes, stems, seeds, ovules, pollen, flowers, fruit, etc.

As used herein, “exogenous nitrogen” refers to non-atmospheric nitrogen readily available in the soil, field, or growth medium that is present under non-nitrogen limiting conditions, including ammonia, ammonium, nitrate, nitrite, urea, uric acid, ammonium acids, etc.

As used herein, “non-nitrogen limiting conditions” refers to non-atmospheric nitrogen available in the soil, field, media at concentrations greater than about 4 mM nitrogen, as disclosed by Kant et al. (2010. J. Exp. Biol. 62 (4): 1499-1509), which is incorporated herein by reference for all purposes.

A “wild type microbe,” e.g., a “wild type bacterium,” as used herein refers to a microbe that has not been genetically modified. Wild type microbes may be isolated and cultivated from a natural source. Wild type microbes may be selected for specific naturally occurring traits.

A “diazotroph” is a microbe that fixes atmospheric nitrogen gas into a more usable form, such as ammonia. A diazotroph is a microorganism that is able to grow without external sources of fixed nitrogen. All diazotrophs contain iron-molybdenum or -vanadium nitrogenase systems.

In some embodiments, the increase of nitrogen fixation and/or the production of 1% or more of the nitrogen in the plant are measured relative to control plants, which have not been exposed to the bacteria of the present disclosure. All increases or decreases in bacteria are measured relative to control bacteria. All increases or decreases in plants are measured relative to control plants.

As used herein, a “water-soluble film package”, interchangeably used herein with “water-soluble package” refers to an encasement that is capable of disintegrating upon contact with a liquid, and is composed of a water-soluble film.

As used herein, an “intergeneric microorganism” is a microorganism that is formed by the deliberate combination of genetic material originally isolated from organisms of different taxonomic genera. An “intergeneric mutant” can be used interchangeably with “intergeneric microorganism”. An exemplary “intergeneric microorganism” includes a microorganism containing a mobile genetic element which was first identified in a microorganism in a genus different from the recipient microorganism. Further explanation can be found, inter alia, in 40 C.F.R. § 725.3.

In aspects, microbes taught herein are “non-intergeneric,” which means that the microbes are not intergeneric.

As used herein, an “intragenic” microorganism, is a microorganism that is engineered to comprise a genetic edit, or genetic modification, or genetic element, or genetic material (e.g. a nucleic acid sequence), that has been sourced from within the organism's own species.

As used herein, a “transgenic” microorganism, is a microorganism that is engineered to comprise a genetic edit, or genetic modification, or genetic element, or genetic material (e.g. a nucleic acid sequence), that has been sourced from outside the organism's taxonomic species.

As used herein, in the context of non-intergeneric microorganisms, the term “remodeled” is used synonymously with the term “engineered”. Consequently, a “non-intergeneric remodeled microorganism” has a synonymous meaning to “non-intergeneric engineered microorganism,” and will be utilized interchangeably. Further, the disclosure may refer to an “engineered strain” or “engineered derivative” or “engineered non-intergeneric microbe,” these terms are used synonymously with “remodeled strain” or “remodeled derivative” or “remodeled non-intergeneric microbe.”

As used herein, “introduced genetic material” means genetic material that is added to, and remains as a component of, the genome of the recipient.

As used herein, the term “engineered” or “engineered microbe” refers to any man-made manipulation of a genome, for example by: insertion, deletion, or substitution of nucleic acid, or manipulation of chromosome number. The manipulation may be induced, random, or targeted, and is synonymous with “genetically engineered.” The engineered manipulation of the genome may occur by any genetic engineering technique provided herein and/or known in the art, e.g. non-limiting examples may include: mutagenesis, mutagenesis induced by selection, mutagenesis induced by chemical mutagens, endonuclease facilitated gene editing, CRISPR, TALEN, ZFNs, homologous recombination, etc.

As used herein, “applying,” “coating,” and “treating” agricultural plant seeds and tissues with the dispersion of microbes and/or biological includes any means by which the plant seeds or tissues are made to come into contact (i.e. exposed) with a dispersion of microbes and/or biological. In some embodiments, “applying” refers to placing or distributing the dispersion of microbes and/or biological onto an area, volume, or quantity of agricultural plant seed or tissue (for example as a seed coat). Consequently, “applying” includes any of the following means of exposure: spraying, dripping, submerging, hand broadcast, machine spreading, brushing, machine broadcasting, and the like, onto agricultural plant seeds and tissues.

As used herein, “application log loss” refers to a measurement of microbial adherence to seed or plant propagating material and is calculated by using the following equation: LOG((Day 0 treatment titers in CFU/ml)×(ml/seed application rate))−LOG(Day 0 CFU/seed). Conceptionally this can be thought of as LOG (of theoretical microbial load per seed as determined by treatment titer and rate of application)−LOG(of actual microbial titer, 25 empirically measured). Lower log loss values indicate higher microbial adherence.

As used herein, when the disclosure discuses a particular microbial deposit by accession number, it is understood that the disclosure also contemplates a microbial strain having all of the identifying characteristics of said deposited microbe, and/or a mutant thereof.

In certain aspects of the disclosure, the isolated microbes exist as “isolated and biologically pure cultures.” It will be appreciated by one of skill in the art, that an isolated and biologically pure culture of a particular microbe, denotes that said culture is substantially free of other living organisms and contains only the individual microbe in question. As used herein, “cultured microorganism” or “cultured microbes” refers to microbes that have been isolated and cultured. The culture can contain varying concentrations of said microbe. The present disclosure notes that isolated and biologically pure microbes often “necessarily differ from less pure or impure materials.” See, e.g. In re Bergstrom, 427 F.2d 1394, (CCPA 1970) (discussing purified prostaglandins), see also, In re Bergy, 596 F.2d 952 (CCPA 1979) (discussing purified microbes), see also, Parke-Davis & Co. v. H.K. Mulford & Co., 189 F. 95 (S.D.N.Y. 1911) (Learned Hand discussing purified adrenaline), aff'd in part, rev'd in part, 196 F. 496 (2d Cir. 1912), each of which are incorporated herein by reference. Furthermore, in some aspects, the disclosure provides for certain quantitative measures of the concentration, or purity limitations, that must be found within an isolated and biologically pure microbial culture. The presence of these purity values, in certain embodiments, is a further attribute that distinguishes the presently disclosed microbes from those microbes existing in a natural state. See, e.g., Merck & Co. v. Olin Mathieson Chemical Corp., 253 F.2d 156 (4th Cir. 1958) (discussing purity limitations for vitamin B12 produced by microbes), incorporated herein by reference.

Microbes of the present disclosure may include spores and/or vegetative cells. In some embodiments, microbes of the present disclosure include microbes in a viable but non-culturable (VBNC) state.

As used herein, a “seed treatment” refers to a substance that may be applied to agricultural seeds. The seed treatment may provide one or more benefits to the seed and/or plant resulting from the seed. Without limitation, seed treatments may include the dispersion of microbes disclosed herein, biologicals disclosed herein, compositions disclosed herein, pesticides, herbicides, insecticides, nematicides, plant-growth promoting factors, fertilizers, and the like. A seed treatment may also be a seed coating.

Certain embodiments of the disclosure refer to seed “pre-treatment.” As used herein the term “pre-treatment” refers to the order of application, where “pre-treatments” are necessarily layered closer to the application locus (e.g., closer to the surface or center of a seed), with subsequent treatments covering over them. Pre-treatment may refer to a single previous seed coating application (e.g. an herbicide), or may be used to collectively refer to all previous treatments adhering to the locus of application (e.g. a seed).

The term “colony forming unit” or “CFU” as used herein is a unit used to estimate the number of viable microbial cells in a sample. Viable is defined as the ability to multiply under the controlled conditions. In some embodiments, counting colony-forming units involves culturing the microbes and counting only viable cells (e.g., cells capable of growing colonies), in contrast with microscopic examination which counts all cells, living or dead.

As used herein, a “chemical buffer,” “buffer solution,” “buffering agent,” or “buffer,” also known as a “pH buffer” or “hydrogen ion buffer,” consists of a mixture of a weak acid and its conjugate base, or a weak base and its conjugate acid.

As used herein, “neutral pH” refers to a pH value of between 6 and 7.5.

As used herein, a “dispersing agent” or “dispersant” is a substance that, when added to a solution or suspension of solid or liquid particles in a liquid, is capable of promoting the separation of the particles and thus, prevent clumping or settling of the particles. In some embodiments, a dispersing agent added to a suspension of microbes disclosed herein can improve and/or stabilize the suspension by promoting the separation of the microbes, and preventing the clumping or settling of the microbes. In some embodiments, addition of a dispersing agent to a dispersion of microbes can promote rehydration, viability, and/or shelf-life of the microbes. In some embodiments, the dispersing agent is a biologically compatible dispersing agent, such as, for example, non-ionic, anionic, amphoteric, or cationic dispersing and emulsifying agents.

As used herein, the term “polymer” includes copolymers. As used herein, “water-soluble polymer” refers to any synthetic, semisynthetic, or natural polymer that dissolves, disperses, or swells in water at least under some conditions, so as to be able to release ingredients admixed with the polymer and/or coated by the polymer into an aqueous solution.

As used herein, “CWT” or “centum weight” is used in the context that is known in the art, as hundredweight for seed. Thus, an amount of treatment per CWT would refer to the amount used to treat 100 pounds of seed.

As used herein, “seed coating” refers to any coating on a seed or plant propagating material.

As used herein, “reconstituted” refers to previously lyophilized microorganisms that have been formulated back to a liquid formulation, but which have not been permitted to grow/culture, since being formulated into the liquid formulation (e.g., through the addition of an aqueous solution). In some embodiments, reconstituted microbes are different from microbes that have not been lyophilized, or which have been allowed to substantially grow/divide (e.g., cultured) since being formulated into liquid suspension.

Embodiments of the present disclosure define compositions based on their % content. In some embodiments the % content is (v/v), which is calculated based on the volume of the recited ingredient divided by the volume of the composition. In some embodiments the % content is (w/v), which is calculated based on the weight (in grams) of the recited ingredient divided by the volume (in liter) of the composition. In some embodiments the % content is (w/w), which is calculated based on the weight of the recited ingredient divided by the weight of the composition.

As used herein, “agricultural biological” or “biological” refers to a category of products derived from naturally occurring microorganisms, plant extracts, or other organic matter.

As used herein, a “biostimulant” refers to a substance or micro-organism that, when applied to seeds, plants, or the rhizosphere, stimulates natural processes within the plant or the plant microbiome (including the entirety of the phytomicrombiome, e.g. the phyllosphere and rhizosphere) to enhance or benefit nutrient uptake, nutrient efficiency, tolerance to abiotic stress, or crop quality and yield.

As used herein, “biochemical pesticides” refers to naturally occurring substances that control pests by non-toxic mechanisms.

As used herein, “microbial pesticides” refer to pesticides having a microorganism (e.g., a bacterium, fungus, virus or protozoan) as the active ingredient.

As used herein, “Plant-Incorporated-Protectants” (PIPs) refers to pesticidal substances that plants produce from genetic material that has been added to the plant.

As used herein, “farm administration system” refers to a system in which the recited elements of the system may be obtained separately from different parties and combined as disclosed herein.

### Overview

The present disclosure provides compositions, kits, and systems comprising engineered bacteria and agricultural biologicals, and methods of use thereof to treat a plant. In some embodiments, the compositions, kits, and systems are effective at improving one or more plant beneficial traits, such as plant yield, nutrient uptake, and resistance to an abiotic or biotic stress. In some embodiments, use of the compositions, kits, and systems disclosed herein decrease usage of fertilizers. The disclosure further teaches methods of using engineered bacteria with agricultural biologicals to treat a plant.

### Agricultural Biologicals

In some embodiments, the present disclosure relates to a composition comprising a plurality of engineered bacteria having at least one genetic variation introduced into at least one gene, or non-coding polynucleotide, of the nitrogen fixation or assimilation genetic regulatory network; and an agricultural biological.

Agricultural biologicals are products that are created from, or derived from, living organisms, plant extracts, beneficial insects, or other organic matter. In recent years they have become a valuable tool in sustainable agriculture. They can be grouped into three main categories: 1) biostimulants, 2) biopesticides, and 3) biofertilizers. Additional names in the art include, for example, bioeffector, biocontrol agent, bioherbicide, bioactivity, biorational insecticide, and biodigester.

### 1) Biostimulants

Biostimulants are substances or microorganisms that, when applied to seeds, plants, or the rhizosphere, stimulates natural processes within the plant or the plant microbiome (including the entirety of the phytomicrombiome, e.g. the phyllosphere and rhizosphere) to enhance or benefit nutrient uptake, nutrient efficiency, tolerance to abiotic stress, or crop quality and yield. In some embodiments, the agricultural biological is a biostimulant. In some embodiments, the biostimulant comprises humic substances, hormones, cell signaling molecules, seaweed extract, and/or amino acids.

In some embodiments, the agricultural biological is a seaweed extract from Reynoutria sachalinensis and/or Ascophyllum nodosum.

In some embodiments, the agricultural biological is selected from auxins, cytokinins, gibberellins, abscisic acid, ethylene, brassinosteroids, jasmonic acid, strigolactones, chemical mimics of strigolactone, and combinations thereof.

In some embodiments, the biostimulant comprises a strigolactone or chemical mimics of strigolactone. Such compounds are described in PCT/US2016/029080, filed Apr. 23, 2016, and entitled: Methods for Hydraulic Enhancement of Crops, and US2021/0329917, published Oct. 28, 2021 and entitled: Compounds and Methods for Increasing Soil Nutrient Availability, which are hereby incorporated by reference. They are further described in U.S. Pat. No. 9,994,557, issued on Jun. 12, 2018, and entitled: Strigolactone Formulations and Uses Thereof, which is hereby incorporated by reference.

In some embodiments, the biostimulant is a compound of Formula (1), a salt, solvate, polymorph, diastereoisomer, stereoisomer, or isomer thereof:

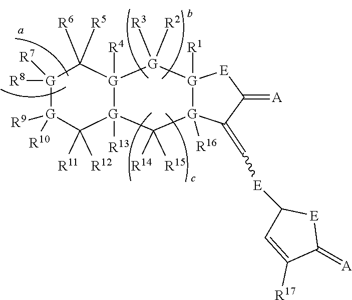

wherein: a, b, and c are one of the following:
   i.a is 0 or 2, and b and c are each independently 0, 1, or 2;
   ii.a is 1, b is 0, and c is 0 or 2;
   iii.a is 1, b is 1, and c is 1 or 2; or
   iv.a is 1, b is 2, and c is 0, 1, or 2;
each A is independently O, or S;
each E is independently O, S, or —NR¹⁸,
each G is independently C;
R⁵, R⁶, R¹¹, R¹², R¹⁴, R¹⁵, and R¹⁷ are each independently H, alkyl, haloalkyl, amino, halo, or —OR¹⁸ or a lone electron pair;
R¹ and R¹⁶ are each independently H, alkyl, haloalkyl, amino, halo, or —OR¹⁸, or R⁴ and R¹³ together form a direct bond to provide a double bond;
Each R¹⁸ is independently H, alkyl, haloalkyl, aryl, heteroaryl, —C(O)R¹⁹ or

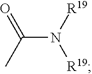

and
each R¹⁹ is independently H, alkyl, haloalkyl, aryl, or heteroaryl.

In some embodiments, the biostimulant is a compound of Formula (2):

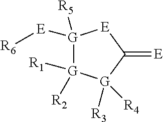

Or any salt, solvate, isomer, or tautomer thereof, wherein:
each E is independently O, S, or —NR₇,
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈,

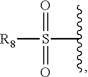

or a lone electron pair, wherein

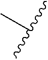

indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R2 and R3 together form a bond, or form a substituted or unsubstituted aryl; and
R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

In some embodiments, the biostimulant is a compound of Formula (3), wherein Formula (3) comprises:

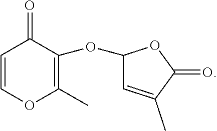 [SMILES: CC1=CC(Oc2c(C)occc2=O)OC1=O]

In some embodiments, the biostimulant comprises a maltol compound. In some embodiments, the biostimulant comprises a lactone compound. In some embodiments, the biostimulant comprises a maltol lactone compound.

In some embodiments, the biostimulant is a commercial product from Table 1.

TABLE 1
Examples of Commercial Biostimulants

| Commercial product | Active ingredients |
|---|---|
| Accomplish ® Max | Ascophyllum nodosum |
| Maritime ® | Ascophyllum nodosum |
| Ferticell ® Calcium 880 Plus | Ascophyllum nodosum |
| Ferticell ® Microelements 1-0-0 | Ascophyllum nodosum |
| Ferticell ® Nutri-Plus 2.5-0-0 | Ascophyllum nodosum |
| Ferticell ® Universal 0-0-1 | Ascophyllum nodosum |
| Norkelp | Ascophyllum nodosum |
| PhycoTerra ® | Ascophyllum nodosum |
| Toggle | Ascophyllum nodosum |
| N-Ext RGS ™ | Ascophyllum nodosum, fulvic acid, humic acids |
| Organic Bactor Boost 0.5-0-0.5 | Ascophyllum nodosum, humic acids, |
| | Azorhizobium Caulinodans, Azotobacter |
| | Chroococcum, Bacillus Subtilis, Bradyrhizobium |
| | Japonicum, Ensifer Meliloti, Enterobacter |
| | Paenibacillus, Brasiliensis Pseudomonas, |
| | Rhizobium Legumin, R. Phaseoli, Ascophyllum |
| | Nodosum, Stenotrophomanas, Trichoderma |
| | Virens, T. Viride |
| Sosdia ™ | Proline and potassium |
| Source ® | Strigolactone |
| Pacesetter | Reynoutria sachalinensis |

### 2) Biopesticides

Biopesticides include any naturally occurring substance that controls pests, known as biochemical pesticides, microorganisms that control pests, known as microbial pesticides, and pesticidal substances produced by plants containing added genetic material-known as plant-incorporated protectants or PIPs. Biopesticides can also include semiochemicals, peptides, proteins and nucleic acids such as double-stranded DNA, single-stranded DNA, double-stranded RNA, single-stranded RNA and hairpin DNA or RNA.

TABLE 2a
Examples of Biopesticides

| Source | Type | Organism | Pest Type |
|---|---|---|---|
| Bacteria | Insecticide | Bacillus thuringiensis | caterpillars, fungi |
| | | var kurstaki | (Botrytis) Elm Leaf |
| | | B. thuringiensis var | Beetle, Alfalfa |
| | | tenebrionis | weevil |
| | Fungicide | Bacillus subtilis | Botrytis spp. |
| Fungi | Insecticide | Beauveria bassiana | Whitefly |
| | Fungicide | Coniothyrium minitans | Sclerotinia spp. |
| | | Trichoderma harzianum | S sclerotiorum |
| | Herbicide | Chondrostereum | cut stumps of |
| | | purpureum | hardwood trees and |
| | | | shrubs |
| | Nematicide | Paecilomyces lilacinus | plant-parasitic |
| | | | nematodes in soil |
| Virus | Insecticide | Cydia pomonella | codling moth |
| | | granulovirus | |
| Oomycetes | Herbicide | Phytophthora palmivora | Morenia orderata |
| Neem (Azadirachta | Insecticide | Azadirachtin | aphids, scale, thrips, |
| indica) | | | whitefly, |
| | | | leafhoppers, weevils |
| Plant extracts | Fungicide | Reynoutria sachalinensis | powdery mildew, |
| | | (giant knotweed) extract | downy mildew, |
| | | | Botrytis, late blight, |
| | | | citrus canker |
| | Herbicide | Plant essential oils | Ragwort, many |
| | | | arthropods |
| | Nematicide | Quillaja saponaria | plant parasitic |
| | | | nematodes |
| Talaromyces flavus; | Biopesticides | | Glomerella |
| Clitoria ternatea | | | cingulata and |
| (butterfly pea); | | | Colletotrichum |
| Trichoderma | | | acutatum; |
| harzianum; Bacillus | | | Helicoverpa spp.; |
| thuringiensis var. | | | Fusarium |
| tenebrionis; | | | oxysporum |
| Lactobacillus casei | | | Agelastica alni; |
| fermentation | | | Spodoptera litura, |
| products | | | Helicoverpa |
| | | | armigera, Aphis |
| | | | gossypii; |
| | | | Xanthomonas |
| | | | fragariae; |
| | | | Spodoptera littoralis |
| | | | and others |
| Semiochemical | Attractant | Citronellol | tetranychid mites |
| | | Multi-component sex | codling moth |
| | | pheromone, such as | |
| | | (E,E)-8,10-dodecadien- | |
| | | 1-ol | |
| Arbuscular | Filamentous | Nostoc piscinale; | |
| Mycorrhizal Fungi | cyanobacterium; | Chlamydopodium | |
| | Single-celled | fusiforme; Chlorella | |
| | green algae | vulgaris | |
| | | Anabaena laxa and | Increase in |
| | | Calothrix elenkinii | fungicidal activity |
| Nanobiopesticide | Silver | | Alternaria alternata, |
| | nanobiopesticide | | A. solani |
| | Sargassum | | Ariadne merione, a |
| | muticum derived | | Lepidopteran pest |
| | NPs | | |
| | Caulerpa | | Culex |
| | scalpelliformis | | quinquefasciatus |
| | and | | |
| | Mesocyclops | | |
| | longisetus- | | |
| | derived NPs | | |

Table 2a adapted from Kumar J, et al., An Overview of Some Biopesticides and Their Importance in Plant Protection for Commercial Acceptance. Plants (Basel). 2021 Jun. 10; 10(6): 1185)

In some embodiments, the agricultural biological is a biochemical pesticide. Biochemical pesticides control pests by non-toxic mechanisms, such as insect sex pheromones that interfere with mating, and plant extracts that attract an insect pest to a trap or repel an insect pest. Examples of plant extracts used as biochemical pesticides are neem and lemongrass oil. A biochemical pesticide may also be an insect growth regulator, and inhibit processes required for survival of the insect.

Plants produce a wide variety of secondary metabolites that deter herbivores from feeding on them. Some of these can be used as biopesticides. They include, for example, pyrethrins, which are fast-acting insecticidal compounds produced by Chrysanthemum cinerariaefolium. They have low mammalian toxicity but degrade rapidly after application. This short persistence prompted the development of synthetic pyrethrins (pyrethroids). The most widely used botanical compound is neem oil, an insecticidal chemical extracted from seeds of Azadirachta indica. Two highly active pesticides are available based on secondary metabolites synthesized by soil actinomycetes, but they have been evaluated by regulatory authorities as if they were synthetic chemical pesticides. Spinosad is a mixture of two macrolide compounds from Saccharopolyspora spinosa. It has a very low mammalian toxicity and residues degrade rapidly in the field. Farmers and growers used it widely following its introduction in 1997 but resistance has already developed in some important pests such as western flower thrips. Abamectin is a macrocyclic lactone compound produced by Streptomyces avermitilis. It is active against a range of pest species but resistance has developed to it also, for example, in tetranychid mites.

Peptides and proteins from a number of organisms have been found to possess pesticidal properties. Perhaps most prominent are peptides from spider venom (King, G. F. and Hardy, M. C. (2013) Spider-venom peptides: structure, pharmacology, and potential for control of insect pests. Annu. Rev. Entomol. 58:475-496). A unique arrangement of disulfide bonds in spider venom peptides render them extremely resistant to proteases. As a result, these peptides are highly stable in the insect gut and hemolymph and many of them are orally active. The peptides target a wide range of receptors and ion channels in the insect nervous system. Other examples of insecticidal peptides include: sea anemone venom that act on voltage-gated Na+ channels (Bosmans, F. and Tytgat, J. (2007) Sea anemone venom as a source of insecticidal peptides acting on voltage-gated Na+ channels. Toxicon. 49 (4): 550-560); the PA1b (Pea Albumin 1, subunit b) peptide from Legume seeds with lethal activity on several insect pests, such as mosquitoes, some aphids and cereal weevils (Eyraud, V. et al. (2013) Expression and Biological Activity of the Cystine Knot Bioinsecticide PAlb (Pea Albumin 1 Subunit b). PLoS ONE 8 (12): e81619); and an internal 10 kDa peptide generated by enzymatic hydrolysis of Canavalia ensiformis (jack bean) urease within susceptible insects (Martinelli, A. H. S., et al. (2014) Structure-function studies on jaburetox, a recombinant insecticidal peptide derived from jack bean (Canavalia ensiformis) urease. Biochimica et Biophysica Acta 1840:935-944). Examples of commercially available peptide insecticides include Spear™-T for the treatment of thrips in vegetables and ornamentals in greenhouses, Spear™-P to control the Colorado Potato Beetle, and Spear™-C to protect crops from lepidopteran pests (Vestaron Corporation, Kalamazoo, MI). A novel insecticidal protein from Bacillus bombysepticus, called parasporal crystal toxin (PC), shows oral pathogenic activity and lethality towards silkworms and Cry1Ac-resistant Helicoverpa armigera strains (Lin, P. et al. (2015) PC, a novel oral insecticidal toxin from Bacillus bombysepticus involved in host lethality via APN and BtR-175. Sci. Rep. 5:11101).

A semiochemical is a chemical signal produced by one organism that causes a behavioral change in an individual of the same or a different species. The most widely used semiochemicals for crop protection are insect sex pheromones, some of which can now be synthesized and are used for monitoring or pest control by mass trapping, lure-and-kill systems and mating disruption. Worldwide, mating disruption is used on over 660,000 ha and has been particularly useful in orchard crops.

In some embodiments, the agricultural biological is a microbial pesticide. Microbial pesticides comprise a microorganism as the active ingredient. The microorganism may be a bacterium, fungus, virus, or protozoan.

An example microbial pesticide are some species and strains of Bacillus thuringiensis (Bt), which can control for example, moths, flies, and mosquitoes. Other microbial pesticides may be obtained from species of Bacillus, Pseudomonas, Yersinia, Chromobacterium, Beauveria, Metarhizium, Verticillium, Lecanicillium, Hirsutella, Paecilomyces, baculoviruses, arbuscular mycorrhizal fungi, Heterorhabditis, and Steinernema. In some embodiments, the microbial pesticide is derived from Bacillus thuringiensis.

The most widely used microbial biopesticide is the insect pathogenic bacteria Bacillus thuringiensis (Bt), which produces a protein crystal (the Bt δ-endotoxin) during bacterial spore formation that is capable of causing lysis of gut cells when consumed by susceptible insects. Microbial Bt biopesticides consist of bacterial spores and 8-endotoxin crystals mass-produced in fermentation tanks and formulated as a sprayable product. Bt does not harm vertebrates and is safe to people, beneficial organisms and the environment. Thus, Bt sprays are a growing tactic for pest management on fruit and vegetable crops where their high level of selectivity and safety are considered desirable, and where resistance to synthetic chemical insecticides is a problem. Bt sprays have also been used on commodity crops such as maize, soybean and cotton, but with the advent of genetic modification of plants, farmers are increasingly growing Bt transgenic crop varieties.

Other microbial insecticides include products based on entomopathogenic baculoviruses. Baculoviruses that are pathogenic to arthropods belong to the virus family and possess large circular, covalently closed, and double-stranded DNA genomes that are packaged into nucleocapsids. More than 700 baculoviruses have been identified from insects of the orders Lepidoptera, Hymenoptera, and Diptera. Baculoviruses are usually highly specific to their host insects and thus, are safe to the environment, humans, other plants, and beneficial organisms. Over 50 baculovirus products have been used to control different insect pests worldwide. In the US and Europe, the Cydia pomonella granulovirus (CpGV) is used as an inundative biopesticide against codlingmoth on apples. Washington State, as the biggest apple producer in the US, uses CpGV on 13% of the apple crop. In Brazil, the nucleopolyhedrovirus of the soybean caterpillar Anticarsia gemmatalis was used on up to 4 million ha (approximately 35%) of the soybean crop in the mid-1990s. Viruses such as Gemstar® (Certis USA) are available to control larvae of Heliothis and Helicoverpa species.

At least 170 different biopesticide products based on entomopathogenic fungi have been developed for use against at least five insect and acarine orders in glasshouse crops, fruit and field vegetables as well as commodity crops. The majority of products are based on the ascomycetes Beauveria bassiana or Metarhizium anisopliae. M. anisopliae has also been developed for the control of locust and grasshopper pests in Africa and Australia and is recommended by the Food and Agriculture Organization of the United Nations (FAO) for locust management.

A number of microbial pesticides are registered in the United States. See for example Kabaluk et al. 2010 (Kabaluk, J. T. et al. (ed.). 2010. The Use and Regulation of Microbial Pesticides in Representative Jurisdictions Worldwide. IOBC Global. 99pp. Microbial pesticides registered in selected countries are listed in Annex 4 of Hoeschle-Zeledon et al. 2013 (Hoeschle-Zeledon, I., P. Neuenschwander and L. Kumar. (2013). Regulatory Challenges for biological control. SP-IPM Secretariat, International Institute of Tropical Agriculture (IITA), Ibadan, Nigeria. 43 pp.), each of which is incorporated herein in its entirety.

In some embodiments, the biopesticide is a commercial product from Table 2b.

TABLE 2b
Examples of Commercial Biopesticides

| Commercial product | Active ingredients |
|---|---|
| Yield Shield | Bacillus pumilus |
| HiStick ® N/T | Bacillus subtilis |
| ActinoGrow ® ST, Actinovate ® | Streptomyces lydicus |
| Poncho ®, Votivo ® | Bacillus firmus |
| RootShield ® WP, PlantShield ® HC | Trichoderma harzianum T-22 |
| SoilGard ® | Gliocladium virens |
| BIO-TAM 2.0 ® | Trichoderma asperellum, Trichoderma gamsii |
| Serenade ®, Cease ® | Bacillus subtilis 713 |
| DoubleNickel ® 55 | Bacillus amyloliquefaciens D747 |
| Sonata ® | Bacillus pumilus 2808 |
| Stargus ™, Amplitude ™ | Bacillus amyloliquefaciens F727 |
| LifeGard ™ WG | Bacillus mycoides isolate J |
| Bexfond ™ | Bacillus amyloliquefaciens subspecies plantarum |
| | strain FZB42 |
| Hearken ™ | Helicoverpa armigera nucleopolyhedrovirus |
| | strain BV-0003 |

In some embodiments, the agricultural biological is a Plant-Incorporated-Protectant (PIP). PIPs are pesticidal substances produced by genetically engineered plants. For example, a plant may be engineered to produce one or more of the pesticidal Cry proteins from Bacillus thuringiensis.

As used herein, “transgenic insecticidal trait” refers to a trait exhibited by a plant that has been genetically engineered to express a nucleic acid or polypeptide that is detrimental to one or more pests. In one embodiment, the plants of the present disclosure are resistant to attach and/or infestation from any one or more of the pests of the present disclosure. In one embodiment, the trait comprises the expression of vegetative insecticidal proteins (VIPs) from Bacillus thuringiensis, lectins and proteinase inhibitors from plants, terpenoids, cholesterol oxidases from Streptomyces spp., insect chitinases and fungal chitinolytic enzymes, bacterial insecticidal proteins and early recognition resistance genes. In another embodiment, the trait comprises the expression of a Bacillus thuringiensis protein that is toxic to a pest. In one embodiment, the Bt protein is a Cry protein (crystal protein). Bt crops include Bt corn, Bt cotton and Bt soy. Bt toxins can be from the Cry family (see, for example, Crickmore et al., 1998, Microbiol. Mol. Biol. Rev. 62:807-812), which are particularly effective against Lepidoptera, Coleoptera and Diptera.

Bt Cry and Cyt toxins belong to a class of bacterial toxins known as pore-forming toxins (PFT) that are secreted as water-soluble proteins undergoing conformational changes in order to insert into, or to translocate across, cell membranes of their host. There are two main groups of PFT: (i) the α-helical toxins, in which α-helix regions form the trans-membrane pore, and (ii) the β-barrel toxins, that insert into the membrane by forming a β-barrel composed of βsheet hairpins from each monomer. See, Parker M W, Feil S C, “Pore-forming protein toxins: from structure to function,” Prog. Biophys. Mol. Biol. 2005 May; 88 (1): 91-142. The first class of PFT includes toxins such as the colicins, exotoxin A, diphtheria toxin and also the Cry three-domain toxins. On the other hand, aerolysin, α-hemolysin, anthrax protective antigen, cholesterol-dependent toxins as the perfringolysin O and the Cyt toxins belong to the β-barrel toxins. Id. In general, PFT producing-bacteria secrete their toxins and these toxins interact with specific receptors located on the host cell surface. In most cases, PFT are activated by host proteases after receptor binding inducing the formation of an oligomeric structure that is insertion competent. Finally, membrane insertion is triggered, in most cases, by a decrease in pH that induces a molten globule state of the protein. Id.

The development of transgenic crops that produce Bt Cry proteins has allowed the substitution of chemical insecticides by environmentally friendly alternatives. In transgenic plants the Cry toxin is produced continuously, protecting the toxin from degradation and making it reachable to chewing and boring insects. Cry protein production in plants has been improved by engineering cry genes with a plant biased codon usage, by removal of putative splicing signal sequences and deletion of the carboxy-terminal region of the protoxin. See, Schuler T H, et al., “Insect-resistant transgenic plants,” Trends Biotechnol. 1998; 16:168-175. The use of insect resistant crops has diminished considerably the use of chemical pesticides in areas where these transgenic crops are planted. See, Qaim M, Zilberman D, “Yield effects of genetically modified crops in developing countries,” Science. 2003 Feb. 7; 299 (5608): 900-2.

Known Cry proteins include: 8-endotoxins including but not limited to: the Cry1, Cry2, Cry3, Cry4, Cry5, Cry6, Cry7, Cry8, Cry9, Cry10, Cry11, Cry12, Cry13, Cry14, Cry15, Cry16, Cry17, Cry18, Cry19, Cry20, Cry21, Cry22, Cry23, Cry24, Cry25, Cry26, Cry27, Cry 28, Cry 29, Cry 30, Cry31, Cry32, Cry33, Cry34, Cry35, Cry36, Cry37, Cry38, Cry39, Cry40, Cry41, Cry42, Cry43, Cry44, Cry45, Cry 46, Cry47, Cry49, Cry 51, Cry52, Cry 53, Cry 54, Cry55, Cry56, Cry57, Cry58, Cry59. Cry60, Cry61, Cry62, Cry63, Cry64, Cry65, Cry66, Cry67, Cry68, Cry69, Cry70 and Cry71 classes of 8-endotoxin genes and the B. thuringiensis cytolytic cyt1 and cyt2 genes.

TABLE 2c
List of exemplary Plant-incorporated Protectants, which
can be combined with microbes of the disclosure

| | | Pesticide |
|---|---|---|
| | Company and Trade | Registration |
| Plant-Incorporated Protectants (PIPs) | Names | Numbers |
| Potato | Potato | |
| Cry3A Potato PC Code 006432 | Naturemark | 524-474 |
| | New Leaf Monsanto | |
| Cry3A & PLRV Potato | Monsanto | 524-498 |
| PC Codes 006432, 006469 | New Leaf Plus | |
| Corn | | |
| Cry1Ab Corn Event 176 PC Code 006458 | Mycogen Seeds/Dow | 68467-1 |
| | Agro | 66736-1 |
| | Syngenta Seeds | |
| Cry1Ab Corn Event Bt11 EPA PC Code | Agrisure CB (with | 67979-1 |
| 006444 OECD Unique Identifier SYN- | Yieldgard) | 65268-1 |
| BTØ11-1, | Attribute Insect | |
| | Protected Sweet Corn | |
| | Syngenta Seeds | |
| Cry1Ab Corn Event MON 801 | Monsanto | 524-492 |
| Cry1Ab corn Event MON 810 PC Code | Monsanto | 524-489 |
| 006430 OECD Unique Identifier MON- | | |
| ØØ81Ø-6 | | |
| Cry1Ac Corn PC Code 006463 | Dekalb Genetics c/o | 69575-2 |
| | Monsanto | |
| | BT-XTRA | |
| Cry1F corn Event TC1507 PC Code | Mycogen Seeds/Dow | 68467-2 |
| 006481 OECD Unique Identifier DAS- | Agro | 29964-3 |
| Ø15Ø7-1 | Pioneer Hi- | |
| | Bred/Dupont | |
| moCry1F corn Event DAS-Ø6275-8 PC | Mycogen Seeds/Dow | 68467-4 |
| Code 006491 OECD Unique Identifier | Agro | |
| DAS-Ø6275-8 | | |
| Cry9C Corn | Aventis | 264-669 |
| | StarLink | |
| Cry3Bb1 corn Event MON863 PC Code | Monsanto | 524-528 |
| 006484 | YielGard RW | |
| OECD Unique Identifier MON-ØØ863-5 | | |
| Cry3Bb1 corn Event MON 88017 PC | Monsanto | 524-551 |
| Code 006498 | YieldGrad VT | |
| OECD Unique Identifier MON-88Ø17-3 | Rootworm | |
| Cry34Ab1/Cry35Ab1 corn Event DAS- | Mycogen Seeds/Dow | 68467-5 |
| 591227-7 | Agro | 29964-4 |
| PC Code 006490 | Pioneer Hi- | |
| OECD Unique Identifier DAS-59122-7 | Bred/Dupont Herculex | |
| | Rootworm | |
| Cry34Ab1/Cry35Ab1 and Cry1F corn | Pioneer Hi- | 29964-17 |
| Event 4114 | Bred/Dupont | |
| PC Codes 006555, 006556 | | |
| mCry3A corn Event MIR 604 | Syngenta Seeds | 67979-5 |
| PC Code 006509 OECD Unique Identifier | Agrisure RW | |
| SYN-IR604-8 | | |
| Cry1A.105 and Cry2Ab2 corn Event | Monsanto | 524-575 |
| MON 89034 PC Codes 006515 and | Genuity VT Double | |
| 006514 | Pro | |
| Vip3Aa20 corn Event MIR 162 | Syngenta Seeds | 67979-14 |
| PC Code 006599 OECD Unique Identifier | Agrisure Viptera | |
| SYN-IR162-4 | | |
| eCry3.1Ab corn in Event 5307 PC Code | Syngenta | 67979-22 |
| 016483 OECD Unique Identifier SYN- | | |
| Æ53Æ7-1 | | |
| Stacked Events and Seed Blend Corn | | |
| MON863 × MON810 with Cry3Bb1 + | Monsanto YieldGard | 524-545 |
| Cry1Ab | Plus | |
| DAS-59122-7 × TC1507 with | Mycogen Seeds/Dow | 68467-6 |
| Cry34Ab1/Cry35Ab1 + Cry1F | Agro Pioneer Hi- | 29964-5 |
| | Bred/Dupont | |
| | Herculex Xtra | |
| MON 88017 × MON 810 with Cry1AB + | Monsanto | 524-552 |
| Cry3Bb | YieldGard VT Triple | |
| | YieldGard VT Plus | |
| MIR 604 × Bt11 with mCry3A + Cry1Ab | Syngenta | 67979-8 |
| | Agrisure CB/RW | |
| | Agrisure 3000GT | |
| Mon 89034 × Mon 88017 with Cry1A.105 + | Monsanto | 524-576 |
| Cry2Ab2 + Cry3Bb1 | Genuity VT Triple | |
| | PRO | |
| Bt11 × MIR 162 with Cry1Ab + | Syngenta Seeds | 67979-12 |
| Vip3Aa20 | Agrisure 2100 | |
| Bt11 × MIR 162 × MIR 604 with Cry1Ab + | Syngenta Seeds | 67979-13 |
| Vip3Aa20 + mCry3A | Agrisure 3100 | |
| MON 89034 × TC1507 × MON 88017 × | Monsanto Company | 524-581 |
| DAS-59122-7 with Cry1A.105 + | Mycogen Seeds/Dow | 68467-7 |
| Cry2Ab2 + Cry1F + Cry3Bb1 + | Agro | |
| Cry34Ab1/Cry35Ab1 | Genuity SmartStax | |
| | SmartStax | |
| MON 89034 × TC1507 × MON 88017 × | Monsanto Company | 524-595 |
| DAS-59122-7 Seed Blend | Mycogen Seeds/Dow | 68467-16 |
| | Agro | |
| | Genuity SmartStax | |
| | RIB Complete | |
| | SmartStax Refuge | |
| | Advanced; Refuge | |
| | Advanced Powered by | |
| | SmartStax | |
| Seed Blend of Herculex Xtra + Herculex I | Pioneer Hi- | 29964-6 |
| | Bred/Dupont | |
| | Optimum AcreMax1 | |
| | Insect Protection | |
| Seed Blend of Herculex RW + Non-Bt | Pioneer Hi- | 29964-10 |
| corn | Bred/Dupont | |
| | Optimum AcreMax | |
| | RW | |
| (Cry1F × Cry34/35 × Cry1Ab) - seed | Pioneer Hi- | 29964-11 |
| blend | Bred/Dupont | |
| | Optimum AcreMax | |
| | Xtra | |
| (Cry1F × Cry1Ab) - seed blend | Pioneer Hi- | 29964-12 |
| | Bred/Dupont | |
| | Optimum AcreMax | |
| | Insect Protection | |
| (Cry1F × mCry3A) | Pioneer Hi- | 29964-13 |
| | Bred/Dupont | |
| | Optimum Trisect | |
| (Cry1F × Cry34/35 × Cry1Ab × mCry3A) | Pioneer Hi- | 29964-14 |
| | Bred/Dupont | |
| | Optimum Intrasect | |
| | Xtreme | |
| 59122 × MON 810 × MIR 604 (Cry34/35 × | Pioneer Hi- | 29964-15 |
| Cry1Ab × mCry3A) | Bred/Dupont | |
| Optimum AcreMax Xtreme (Cry1F × | Pioneer Hi- | 29964-16 |
| Cry34/35 × Cry1Ab × mCry3A) - seed | Bred/Dupont | |
| blend | Optimum AcreMax | |
| | Xtreme (seed blend) | |
| MON 810 × MIR 604 (Cry1Ab × | Pioneer Hi- | 29964-18 |
| mCry3A) | Bred/Dupont | |
| 1507 × MON810 × MIR 162 (Cry1F × | Pioneer Hi- | 29964-19 |
| Cry1Ab × Vip3Aa20) | Bred/Dupont | |
| | Optimum Intrasect | |
| | Leptra | |
| 1507 × MIR 162 (Cry1F × Vip30Aa20) | Pioneer Hi- | 29964-20 |
| | Bred/Dupont | |
| 4114 × MON 810 × MIR 604 (Cry34/35 × | Pioneer Hi- | 29964-21 |
| Cry1F × Cry1Ab × mCry3A) - seed blend | Bred/Dupont | |
| 4114 × MON 810 × MIR 604 (Cry34/35 × | Pioneer Hi- | 29964-22 |
| Cry1F × Cry1Ab × mCry3A) | Bred/Dupont | |
| 1507 × MON810 × MIR 604 (Cry1F × | Pioneer Hi- | 29964-23 |
| Cry1Ab × mCry3A) - seed blend | Bred/Dupont | |
| | Optimum AcreMax | |
| | Trisect | |
| 1507 × MON810 × MIR 604 (Cry1F × | Pioneer Hi- | 29964-24 |
| Cry1Ab × mCry3A) | Bred/Dupont | |
| | Optimum Intrasect | |
| | Trisect | |
| 4114 × MON 810 (Cry34/35 × Cry1F × | Pioneer Hi- | 29964-25 |
| Cry1Ab) | Bred/Dupont | |
| 1507 × MON810 × MIR 162 (Cry1F × | Pioneer Hi- | 29964-26 |
| Cry1Ab × Vip3Aa20) - seed blend | Bred/Dupont | |
| | Optimum AcreMax | |
| | Leptra | |
| SmartStax Intermediates (8 products) | Monsanto | 524-583, 524-584, |
| | | 524-586, 524 -587, |
| | | 524-588, 524-589, 524-590 |
| MON 89034 × 1507 (Cry1A.105 × | Monsanto | 524-585 |
| Cry2Ab2 × Cry1F) | Genuity PowerCore | |
| MON 89034 (Cry1A.105 × Cry2Ab2) - | Monsanto | 524-597 |
| seed blend | Genuity VT Double | |
| | PRO RIB Complete | |
| MON 89034 × 88017 RIB Complete | Monsanto | 524-606 |
| (Cry1A.105 × Cry2Ab2 × Cry3Bb1) - | Genuity VT Triple | |
| seed blend | PRO RIB Complete | |
| MON 89034 × 1507 (Cry1A.105 × | Monsanto | 524-612 |
| Cry2Ab2 × Cry1F) - seed blend | Genuity PowerCore | |
| | RIB Complete | |
| Bt11 × MIR 162 × 1507 (Cry1Ab × | Syngenta Seeds | 67979-15 |
| Vip3Aa20 × Cry1F) | Agrisure Viptera 3220 | |
| | Refuge Renew | |
| Bt11 × 59122-7 × MIR 604 × 1507 | Syngenta Seeds | 67979-17 |
| (Cry1Ab × Cry34/35 × mCry3A × Cry1F) | Agrisure 3122 | |
| Bt11 × MIR 162 × TC1507 (Cry1Ab × | Syngenta Seeds | 67979-19 |
| Vip3Aa20 × Cry1F) - seed blend | Agisure Viptera 3220 | |
| | (E-Z Refuge) (Refuge | |
| | Advanced) | |
| Bt11 × DAS 59122-7 × MIR604 × | Syngenta Seeds | 67979-20 |
| TC1507 (Cry1Ab × Cry34/35 × mCry3A × | Agisure Viptera 3122 | |
| Cry1F) - seed blend | (E-Z Refuge) (Refuge | |
| | Advanced) | |
| Bt11 × MIR 162 × MIR 604 × TC1507 × | Syngenta Seeds | 67979-23 |
| 5307 (Cry1Ab × Vip3Aa20 × mCry3A × | Agrisure Duracade | |
| Cry1F × eCry3.1Ab) | (Refuge Renew) 5222 | |
| Bt11 × MIR 604 × TC1507 × 5307 | Syngenta Seeds | 67979-24 |
| (Cry1Ab × mCry3A x Cry1F × | Agrisure Duracade | |
| eCry3.1Ab) | (Refuge Renew) 5122 | |
| Bt11 × MIR 604 × TC1507 × 5307 | Syngenta Seeds | 67979-25 |
| (Cry1Ab × mCry3A × Cry1F × | Agisure Duracade | |
| eCry3.1Ab) - seed blend | 5122 E-Z Refuge | |
| Bt11 × MIR 162 × MIR 604 × TC1507 × | Syngenta Seeds | 67979-26 |
| 5307 (Cry1Ab × Vip3Aa20 × mCry3A × | Agisure Duracade | |
| Cry1F × eCry3.1Ab) - seed blend | 5222 E-Z Refuge | |
| Bt11 × MIR 162 × MIR 604 × TC1507 × | Syngenta Seeds | 67979-27 |
| 5307 (Cry1Ab × Vip3Aa20 × mCry3A × | Agrisure Duracade | |
| Cry1F × eCry3.1Ab) | (Refuge Renew) 5022 | |
| MIR604 × DAS-59122-7 × TC1507 | Syngenta Seeds | 67979-29 |
| (mCry3A × Cry34/35 × Cry1F) | | |
| SmartStax Intermediates (8 products) | Mycogen Seeds/Dow | 68467-8, 68467-9, |
| | Agro | 68467-10, 68467-11, |
| | | 68467-13, 68467-14, |
| | | 68467-15 |
| MON 89034 × 1507 (Cry1A.105 × | Mycogen Seeds/Dow | 68467-12 |
| Cry2Ab2 × Cry1F) | Agro | |
| | PowerCore; | |
| | PowerCore Enlist | |
| MON 89034 × 1507 (Cry1A.105 × | Mycogen Seeds/Dow | 68467-21 |
| Cry2Ab2 × Cry1F) - seed blend | Agro | |
| | PowerCore Refuge | |
| | Advanced; Refuge | |
| | Advanced Powered by | |
| | PowerCore | |
| 1507 × MON 810 | Pioneer Hi- | 29964-7 |
| | Bred/Dupont | |
| | Optimum Intrasect | |
| 59122 × 1507 × MON 810 | Pioneer Hi- | 29964-8 |
| | Bred/Dupont | |
| 59122 × MON 810 | Pioneer Hi- | 29964-9 |
| | Bred/Dupont | |
| Cotton | | |
| Cry1Ac Cotton | Monsanto | 524-478 |
| | BollGard | |
| Cry1Ac and Cry2Ab2 in Event 15985 | Monsanto | 524-522 |
| Cotton PC Codes 006445, 006487 | BollGard II | |
| Bt cotton Event MON531 with Cry1Ac | Monsanto | 524-555 |
| (breeding nursery use only) | | |
| Bt cotton Event MON15947 with | Monsanto | 524-556 |
| Cry2Ab2 (breeding nursery use only) | | |
| COT102 × MON 15985 (Vip3Aa19 × | Monsanto | 524-613 |
| Cry1Ac × Cry2Ab2) | Bollgard III | |
| Cry1F and Cry1Ac (Events DAS-21023-5 × | Mycogen Seeds/Dow | 68467-3 |
| DAS-24236-5) Cotton PC Codes | Agro | |
| 006512, 006513 | Widestrike | |
| Event 3006-210-23 (Cry1Ac) | Mycogen Seeds/Dow | 68467-17 |
| | Agro | |
| Event 281-24-236 (Cry1F) | Mycogen Seeds/Dow | 68467-18 |
| | Agro | |
| WideStrike × COT102 (Cry1F × Cry1Ac × | Mycogen Seeds/Dow | 68467-19 |
| Vip3Aa19) | Agro | |
| | WideStrike 3 | |
| Vip3Aa19 and FLCry1Ab (Events | Syngenta Seeds | 67979-9 |
| Cot102 × Cot67B) Cotton PC Codes | (Formally VipCot) | |
| 016484, 016486 OECD Unique Identifier | | |
| SYN-IR102-7 × SYN-IR67B-1 | | |
| COT102 (Vip3Aa19) | Syngenta Seeds | 67979-18 |
| COT67B (FLCry1Ab) | Syngenta Seeds | 67979-21 |
| T304-40 (Cry1Ab) | Bayer CropScience | 264-1094 |
| GHB119 (Cry2Ae) | Bayer CropScience | 264-1095 |
| T304-40 × GHB 119 (Cry1Ab × Cry2Ae) | Bayer CropScience | 264-1096 |
| OECD Unique Identifier: BCS-GHØØ4-7 × | TwinLink | |
| BCS-GHØØ5-8 | | |
| Soybean | | |
| Cry1Ac in Event 87701 Soybean PC | Monsanto | 524-594 |
| Code 006532 OECD Unique Identifier | Inacta | |
| Cry1A.105 and Cry2Ab2 in Event 87751 | Monsanto | 524-619 |
| Soybean PC Codes 006614, 006615 | | |
| OECD Unique Identifier MON-87751-7 | | |
| Cry1Ac × Cry1F in Event DAS 81419 | Mycogen Seeds/Dow | 68467-20 |
| Soybean PC Codes 006527, 006528 | Agro | |
| OECD Unique Identifier | | |
| DAS 81419 (Cry1Ac × Cry1F) | | |

### 3) Biofertilizers

Biofertilizers are microorganisms, such as bacteria, fungi, and algae, that provide plants with nutrients, or help them to absorb nutrients, thus improving plant yield. Types of biofertilizers include, but are not limited to, nitrogen fixation, phosphate solubilization, nutrient mobilization, plant growth-promoting bacteria, and plant growth-regulating bacteria.

In some embodiments, the agricultural biological is a biofertilizer selected from the group consisting of a bacterial, algal, and fungal biofertilizer. In some embodiments, the agricultural biological is a biofertilizer that comprises at least one of a nitrogen fixer, a phosphate solubilizer, a nutrient mobilizer, plant growth-promoting bacteria, and plant growth-regulating bacteria.

In some embodiments, the agricultural biological comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizoctonia, Rhizobium, Rhodopseudomonas, Sinorhizobium, Trichoderma, and combinations thereof.

In some embodiments, the agricultural biological comprises plant growth-promoting fungi and/or plant growth-promoting bacteria.

### Plant Growth-Promoting Fungi (PGPF)

PGPF species are beneficial to plants in several ways. For example, they can solubilize and mineralize nutrients making them accessible to plants; they regulate hormones; they produce compounds that suppress plant pathogens and alleviate abiotic stressors. PGPR species have been identified in species of the genera Aspergillus, Penicillium, Phoma, Fusarium, Trichoderma, Piriforma, and Glomus.

### Aspergillus

Species of the fungi Aspergillus can protect plants and promote plant growth via production of pytases, auxins, gibberellins, and many secondary metabolites. The phytases for example, aid in phosphate solubilization. Some species of Aspergillus also are antagonist to plant pathogens (see for example, Nayak S. et al., (2020). Beneficial Role of Aspergillus sp. in Agricultural Soil and Environment, Frontiers in Soil and Environmental Microbiology (pp. 17-36)). Species of Aspergillus that have plant beneficial activity that may be included with the compositions, methods, kits, and systems disclosed herein include, but are not limited to, A. aculeatus, A. brasiliensis, A. clavatus, A. flavus, A. fumigatus, A. mellus, A. niger, A. nidulans, A. oryzae, A. sydowii, A. terreus, A. tubingensis, A. ustus, and A. sp. versicolor.

### Penicillium

Many species of Penicillium have positive interactions with plants and can promote plant growth by supplying soluble phosphorus, indole-3-acetic acid, and gibberellic acid, and can also provide protection by acting as an antagonist to pathogens and/or activating plant defense signaling, and tolerance to abiotic stressors related to temperature, heavy metals, salt, and water. In some embodiments, the agricultural biological comprises a species of Penicillium selected from P. bilaiae. P. brevicompactum, P. brocae, P. canescens, P. cecidicola, P. citrinum, P. coffeae, P. commune, P. crustosum, P. funiculosum, P. janthinellum, P. monteilii, P. olsonii, P. oxalicum, P. radicum, P. ruqueforti, P. sclerotiorum, P. simplicissimum, and P. steckii

### Trichoderma

Species of Trichoderma are present in soils all over the world. They have been shown to form mutualistic relationships with several plant species, regulating the rate of plant growth and suppressing the growth of plant pathogens through competition, antibiotic production, and chitinase secretion. The fungi further secrets organic acids that solubilize phosphates and mineral ions, such as iron, magnesium, and manganese. In some embodiments, the agricultural biological comprises a species of Trichoderma selected from T. harzianum, T. atroviride, T. asperellum, T. virens, T longipile, T. tomentosum, T. viride, T. afroharzianum, and T. hamatum.

### Mycorrhizal Fungi and Glomus

Mycorrhizal fungi enhance plant access to soil nutrients and water. There are two functional types, arbuscular mycorrhizal fungi (AMF) and ectomycorrhizal fungi (EMF) which partner with plants having different nutrient acquisition strategies (for example, fast N cycling vs. slow N cycling). An example genus of AMF is Glomus. In some embodiments, the agricultural biological is a mycorrhizal fungi selected from Glomus intraradices, Glomus mosseae, Glomus aggregatum, Glomus etunicatum, Glomus clarus, and Rhizophagus intraradices.

### Plant Growth-Promoting Rhizobacteria (PGPR)

PGPR species promote plant growth via direct mechanisms (for example, by improving nutrient acquisition and regulating phytohormones) and indirect mechanisms (for example, by inducing resistance to stressors or competing with a pathogen). PGPR species have been identified in species of the genera Acinetobacter, Aeromonas, Agrobacterium, Allorhizobium, Arthrobacter, Azoarcus, Azorhizobium, Azospirillum, Azotobacter, Bacillus, Bradyrhizobium, Burkholderia, Caulobacter, Chromobacterium, Delftia, Enterobacter, Flavobacterium, Frankia, Gluconacetobacter, Klebsiella, Mesorhizobium, Methylobacterium, Micrococcus, Paenibacillus, Pantoea, Pseudomonas, Rhizobium, Serratia, Streptomyces, and Thiobacillus.

### Azospirillum

Azospirillum species have been shown to increase the yield, drought tolerance, and salt tolerance of crops such as corn, wheat, rice, and sugarcane (see for example G. F. Vogel, et al., Agronomic performance of Azospirillum brasilense on wheat crops, Appl. Res. Agrotechnol., 6 (2013), pp. 111-119; J. E. Garcia, et al., In vitro PGPR properties and osmotic tolerance of different Azospirillum native strains and their effects on growth of maize under drought stress, Microbiological Research 202 (2017) pp 21-29). Azospirillum further promotes plant growth through production of auxins, cytokinins, and gibberellins. In some embodiments, the agricultural biological comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

### Pseudomonas

Pseudomonas species are present in both the rhizosphere as well as the within plant tissues. They have been extensively studied for their roles in plant growth promotion, control of pests and pathogens, and nutrient solubilization (Kumar A., et al., Role of Pseudomonas sp. in Sustainable Agriculture and Disease Management, (2017) pp 195-215). In some embodiments, the agricultural biological comprises a species of Pseudomonas selected from P. aeruginosa, P. aureofaciens, P. cepacia (formerly known as Burkholderia cepacia), P. chlororaphis, P. corrugata, P. fluorescens, P. proradix, P. putida, P. rhodesiae, P. syringae, P. protegens, P. chlororaphis, P. segetis, and P. segetis strain P6.

### Bacillus

Bacillus is a diverse group of bacteria in the soil ecosystem, playing roles in nutrient cycling and imparting plant beneficial traits such as stress tolerance (see for example A. K. Saxena et al., “Bacillus species in soil as a natural resource for plant health and nutrition.” 2019. J of App. Microbiology, 128 (5): 1583-1594). In some embodiments, the agricultural biological comprises a species of Bacillus selected from B. subtilis, B. velezensis, B. siamensis, B. cereus, B. thuringiensis, B. thuringiensis subsp. israelensis, B. thuringiensis subsp. tenebrionis strain SA-10, B. thuringiensis subsp. aizawai, Bacillus thuringiensis strain VBTS 2528, B. licheniformis, B. pumilus, Bacillus pumilus strain QST 2808, B. altitudinis, B. stratosphericus, B. aerius, B. safensis, B. australimaris, B. amyloliquefaciens, B. methylotrophicus, B. megaterium, B. simplex, B. sp. AQ175 (ATCC Accession No. 55608), B. sp. AQ 177 (ATCC Accession No. 55609), B. sp. AQ178 (ATCC Accession No. 53522), B. sphaericus, B. bombysepticus, B. firmus, B. coagulans, B. azotofixans, and B. macerans.

### Methylobacterium

Methylobacterium are a genus of non-pathogenic bacteria found in a wide range of environments. A number of species of Methylobacterium have been shown to promote plant growth through their production of plant hormones such as cytokinins, abscisic acid, and indole-3-acetic acid. Of note, they are able to produce high levels of cytokinins and the active trans-Zeatin form (see for example, Palberg, D., et al. A survey of Methylobacterium species and strains reveals widespread production and varying profiles of cytokinin phytohormones. BMC Microbiol 22, 49 (2022)). In some embodiments, the agricultural biological is a species of Methylobacterium selected from M. gregans, M. adhaesivum, M. aerolatum, M. ajmalii, M. aquaticum, M. aminovorans, M. brachiatum, M. brachythecii, M. bullatum, M. cerastii, M. crusticola, M. currus, M. dankookense, M. durans, M. extorquens, M. frigidaeris, M. fujisawaense, M. funariae, M. gnaphalii, M. goesingense, M. gossipicola, M. haplocladii, M. hispanicum, M. indicum, M. iners, M. isbiliense, M. jeotgali, M. komagatae, M. longum, M. marchantiae, M. mesophylicum, M. nodulans, M. nonmethylotrophicum, M. organophillum, M. oryzae, M. oryzihabitans, M. oxalidis, M. persicinum, M. phyllosphaerae, M. phyllostachyos, M. planium, M. platani, M. pseudosasicola, M. radiotolerans corrig., M. rhodinum, M. segetis, M. soli, M. symbioticum, M. tardum, M. tarhaniae, M. terrae, M. terricola, M. thuringiense, M. trifolii, M. thiyocyanatum, M. variabile, M. zatmanii.

### Gluconacetobacter

Species of Gluconacetobacter can establish symbiotic relationships with plants and promote growth and nitrogen fixation. In some embodiments, the agricultural biological is a species of Gluconacetobacter selected from G. azotocaptans, G. diazotrophicus, G. johannae, and G. sacchari

### Combinations of Different Species of Fungi and Bacteria

In some embodiments, the agricultural biological is a combination of different species of bacteria, fungi and algae.

In some embodiments, the agricultural biological comprises a species of Pseudomonas and Bacillus.

In some embodiments, the agricultural biological comprises Azotobacter vinelandii and Clostridium pasteurianum.

In some embodiments, the agricultural biological comprises a mycorrhizal fungi, Beauveria bassiana, Azospirillum sp., Azotobacter sp., and Rhodopseudomonas palustris.

In some embodiments, the agricultural biological comprises a mycorrhizal fungi, Clostridium sp., and Azotobacter sp.

In some embodiments, the agricultural biological comprises Bacillus amyloliquefaciens and Trichoderma virens.

In some embodiments, the agricultural biological comprises Ochrobactrum anthropic, Bacillus Subtillus, and Bacillus simplex.

In some embodiments, the biofertilizer is a commercial product from Table 3.

TABLE 3
Example Commercial Biofertilizers

| Commercial product | Active ingredients |
|---|---|
| Accomplish ® LM | Bacillus pumilus, B. megaterium, |
| | B. licheniformis |
| SoilBuilder | Bacillus licheniformis |
| Titan XC | Bacillus licheniformis |
| Rootella ®X | Glomus intraradices |
| Terrasym 450 | Methylotrophs, Methylobacterium |
| | gregans |
| Envita | Gluconacetobacter diazotrophicus |
| iNvigorate | Azotobacter vinelandii, Clostridium |
| | pasteurianum |
| PrimAgro N | Bacillus subtilis |
| EndoFuse | Glomus intraradices, G. mosseae, G. |
| | aggregatum, G. etunicatum |
| MycoGold | Beauveria Bassiana, Azospirillum, Azotobacter |
| BioFlex | Azotobacter, Clostridium, Mycorrhizae |
| Rhyzo-link | Bacillus subtilis, Bacillus methylotrophicus, |
| | Bacillus amyloliquefaciens, Bacillus |
| | megaterium, Bacillus licheniformis |
| QuickRoots | Bacillus amyloliquefaciens, and Trichoderma |
| | virens |
| SabrEx for Corn | Trichoderma |
| Biotrinsic M33 FP + M34 FP and W10 | Trichoderma harzianum, Trichoderma atroviride |
| MicroAZ | Ochrobactrum anthropi and Bacillus Subtilis, |
| | Bacillus simplex |
| JumpStart | Azospirillum brasilensis |
| Utrisha ™ | Methylobacterium symbioticum SB23 |

### Engineered Microbes

In some embodiments, the microbes are genetically engineered. In some embodiments, the microbes are non-intergeneric remodeled microbes. In some embodiments, the microbes are transgenic. The term “non-intergeneric” indicates that the genetic variations introduced into the host do not contain nucleic acid sequences from outside the host genus. In some embodiments, the microbes are intragenic. Therefore, in some embodiments, the microbes are not transgenic. For example, for non-transgenic microbes with varied promoters, promoters for promoter swapping are selected from within the microbe's genome, or genus.

Microbes may be obtained from any general terrestrial environment, including its soils, plants, fungi, animals (including invertebrates) and other biota, including the sediments, water and biota of lakes and rivers; from the marine environment, its biota and sediments (for example, sea water, marine muds, marine plants, marine invertebrates (for example, sponges), marine vertebrates (for example, fish); the terrestrial and marine geosphere (regolith and rock, for example, crushed subterranean rocks, sand and clays); the cryosphere and its meltwater; the atmosphere (for example, filtered aerial dusts, cloud and rain droplets); urban, industrial and other man-made environments (for example, accumulated organic and mineral matter on concrete, roadside gutters, roof surfaces, and road surfaces).

The plants from which the microbes are obtained may be a plant having one or more desirable traits, for example a plant which naturally grows in a particular environment or under certain conditions of interest. By way of example, a certain plant may naturally grow in sandy soil or sand of high salinity, or under extreme temperatures, or with little water, or it may be resistant to certain pests or disease present in the environment, and it may be desirable for a commercial crop to be grown in such conditions, particularly if they are, for example, the only conditions available in a particular geographic location. By way of further example, the bacteria may be collected from commercial crops grown in such environments, or more specifically from individual crop plants best displaying a trait of interest amongst a crop grown in any specific environment: for example the fastest-growing plants amongst a crop grown in saline-limiting soils, or the least damaged plants in crops exposed to severe insect damage or disease epidemic, or plants having desired quantities of certain metabolites and other compounds, including fiber content, oil content, and the like, or plants displaying desirable colors, taste or smell. The bacteria may be collected from a plant of interest or any material occurring in the environment of interest, including fungi and other animal and plant biota, soil, water, sediments, and other elements of the environment as referred to previously.

The microbe may be isolated from plant tissue. This isolation can occur from any appropriate tissue in the plant, including for example root, stem and leaves, and plant reproductive tissues. Non-limiting examples of plant tissues include a seed, seedling, leaf, cutting, plant, bulb, tuber, root, and rhizomes. In some embodiments, microorganisms are isolated from a seed. In some embodiments, microorganisms are isolated from a root.

Persons having skill in the art will be familiar with techniques for recovering microbes from various environmental sources. For example, microbes useful in the compositions and methods disclosed herein can be obtained by extracting microbes from surfaces or tissues of native plants; grinding seeds to isolate microbes; planting seeds in diverse soil samples and recovering microbes from tissues; or inoculating plants with exogenous microbes and determining which microbes appear in plant tissues. The parameters for processing samples may be varied to isolate different types of associative microbes, such as rhizospheric, epiphytes, or endophytes. By way of example, some methods for isolation from plants include the sterile excision of the plant material of interest (e.g. root or stem lengths, leaves), surface sterilization with an appropriate solution (e.g. 2% sodium hypochlorite), after which the plant material is placed on nutrient medium for microbial growth. Alternatively, the surface-sterilized plant material can be crushed in a sterile liquid (usually water) and the liquid suspension, including small pieces of the crushed plant material spread over the surface of a suitable solid agar medium, or media, which may or may not be selective (e.g. contain only phytic acid as a source of phosphorus). This approach is especially useful for bacteria which form isolated colonies and can be picked off individually to separate plates of nutrient medium, and further purified to a single species by well-known methods. Alternatively, the plant root or foliage samples may not be surface sterilized but only washed gently thus including surface-dwelling epiphytic microorganisms in the isolation process, or the epiphytic microbes can be isolated separately, by imprinting and lifting off pieces of plant roots, stem or leaves onto the surface of an agar medium and then isolating individual colonies as above. This approach is especially useful for bacteria, for example. Alternatively, the roots may be processed without washing off small quantities of soil attached to the roots, thus including microbes that colonize the plant rhizosphere. Otherwise, soil adhering to the roots can be removed, diluted and spread out onto agar of suitable selective and non-selective media to isolate individual colonies of rhizospheric bacteria.

Microbes may also be sourced from a repository, such as environmental strain collections, instead of initially isolating from a first plant. The microbes can be genotyped and phenotyped, via sequencing the genomes of isolated microbes; profiling the composition of communities in planta; characterizing the transcriptomic functionality of communities or isolated microbes; or screening microbial features using selective or phenotypic media (e.g., nitrogen fixation or phosphate solubilization phenotypes). Selected candidate strains or populations can be obtained via sequence data; phenotype data; plant data (e.g., genome, phenotype, and/or yield data); soil data (e.g., pH, N/P/K content, and/or bulk soil biotic communities); or any combination of these

In some embodiments, the engineered microbes are selected from species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof. In some aspects, the engineered microbes are non-intergeneric. In some aspects, the engineered microbes are transgenic. In some aspects, the non-intergeneric remolded microbes comprise Kosakonia sacchari. In some aspects, the non-intergeneric remolded microbe is Kosakonia sacchari PTA-126743, as described in International Patent Publication No. WO2021222567. In some aspects, the non-intergeneric remolded microbe is Klebsiella variicola. In some aspects, non-intergeneric remolded microbe is Klebsiella variicola PTA-126740, as described in International Patent Publication No. WO2021222567.

While Kosakonia sacchari strain PTA-126743 and Klebsiella variicola strain PTA-126740 were used in the experiments disclosed herein, it will be understood by one skilled in the art that any type of engineered microbe may be used with the compositions, methods, kits, and systems disclosed herein. Non-limiting examples of other beneficial microbes are provided below in Table 4, for example.

TABLE 4
Microorganisms Deposited under the Budapest Treaty

| | Alternate | | | |
|---|---|---|---|---|
| Depository | designations | Taxonomy | Accession Number | Date of Deposit |
| NCMA | CI006, | Kosakonia sacchari | NCMA 201701001 | Jan. 6, 2017 |
| | PBC6.1 | (WT) | | |
| NCMA | CI019 | Rahnella aquatilis (WT) | NCMA 201701003 | Jan. 6, 2017 |
| NCMA | CM029 | Kosakonia sacchari | NCMA 201701002 | Jan. 6, 2017 |
| NCMA | CM037 | Kosakonia sacchari | NCMA 201708004 | Aug. 11, 2017 |
| NCMA | CM38, | Kosakonia sacchari | NCMA 201708003 | Aug. 11, 2017 |
| | PBC6.38 | | | |
| NCMA | CM094, | Kosakonia sacchari | NCMA 201708002 | Aug. 11, 2017 |
| | PBC6.94 | | | |
| NCMA | CI137, 137, | Klebsiella variicola | NCMA 201708001 | Aug. 11, 2017 |
| | PB137 | (WT) | | |
| NCMA | | Klebsiella variicola | NCMA 201712001 | Dec. 20, 2017 |
| NCMA | | Klebsiella variicola | NCMA 201712002 | Dec. 20, 2017 |
| ATCC | | Kosakonia sacchari | PTA-126575 | Dec. 23, 2019 |
| ATCC | | Kosakonia sacchari | PTA-126576 | Dec. 23, 2019 |
| ATCC | | Klebsiella variicola | PTA-126577 | Dec. 23, 2019 |
| ATCC | | Klebsiella variicola | PTA-126578 | Dec. 23, 2019 |
| ATCC | | Klebsiella variicola | PTA-126579 | Dec. 23, 2019 |
| ATCC | | Klebsiella variicola | PTA-126580 | Dec. 23, 2019 |
| ATCC | | Paenibacillus polymyxa | PTA-126581 | Dec. 23, 2019 |
| | | (WT) | | |
| ATCC | CI8 | Paraburkholderia | PTA-126582 | Dec. 23, 2019 |
| | | tropica (WT) | | |
| ATCC | CI3069 | Herbaspirillum | PTA-126583 | Dec. 23, 2019 |
| | | aquaticum (WT) | | |
| ATCC | | Metakosakonia intestini | PTA-126584 | Dec. 23, 2019 |
| ATCC | CI910 | Metakosakonia intestini | PTA-126585 | Dec. 23, 2019 |
| | | (WT) | | |
| ATCC | | Metakosakonia intestini | PTA-126586 | Dec. 23, 2019 |
| ATCC | | Metakosakonia intestini | PTA-126587 | Dec. 23, 2019 |
| ATCC | | Metakosakonia intestini | PTA-126588 | Dec. 23, 2019 |
| ATCC | | Klebsiella variicola | PTA-126740 | Mar. 25, 2020 |
| ATCC | | Kosakonia sacchari | PTA-126743 | Mar. 25, 2020 |

In some aspects, the engineered microbes of the disclosure are those from Table 4. In other aspects, the engineered microbes of the disclosure are derived from a microorganism of Table 4. For example, a strain, child, mutant, or derivative, of a microorganism from Table 4 are provided herein. The disclosure contemplates all possible combinations of microbes listed in Table 4, said combinations sometimes forming a microbial consortia. The microbes from Table 4, either individually or in any combination, can be combined with any plant, active molecule (synthetic, organic, etc.), adjuvant, carrier, supplement, biofilm, or biological in a microbial composition. In some aspects, the engineered microbes are a microbial composition comprising at least one of a polymer, sugar, biofilm, and isolated biofilm compositions. 10

In some embodiments, the engineered microbes of this disclosure are nitrogen fixing microbes, for example nitrogen fixing bacteria, nitrogen fixing archaea, nitrogen fixing fungi, nitrogen fixing yeast, nitrogen fixing algae, or nitrogen fixing protozoa. In some aspects, microbes useful in the compositions and methods disclosed herein are spore forming microbes, for example spore forming bacteria. In some aspects, bacteria useful in the compositions and methods disclosed herein are Gram positive bacteria or Gram negative bacteria. In some embodiments, the bacteria are endospore forming bacteria of the Firmicute phylum. In some embodiments, the bacteria are diazotrophs. In some embodiments, the bacteria are not diazotrophs.

In some embodiments, the compositions, methods, kits, and systems of the disclosure are used with an archaea, such as, for example, Methanothermobacter thermoautotrophicus, Methanosarcina barkeri, Methanospirillum hungatei, Methanobacterium bryantii, Methanococcus thermolithotrophicus, and Methanococcus maripaludis.

In some embodiments, the engineered microbes include, but are not limited to, Agrobacterium radiobacter, Bacillus acidocaldarius, Bacillus acidoterrestris, Bacillus agri, Bacillus aizawai, Bacillus albolactis, Bacillus alcalophilus, Bacillus alvei, Bacillus aminoglucosidicus, Bacillus aminovorans, Bacillus amylolyticus (also known as Paenibacillus amylolyticus) Bacillus amyloliquefaciens, Bacillus aneurinolyticus, Bacillus atrophaeus, Bacillus azotoformans, Bacillus badius, Bacillus cereus (synonyms: Bacillus endorhythmos, Bacillus medusa), Bacillus chitinosporus, Bacillus circulans, Bacillus coagulans, Bacillus endoparasiticus Bacillus fastidiosus, Bacillus firmus, Bacillus kurstaki, Bacillus lacticola, Bacillus lactimorbus, Bacillus lactis, Bacillus laterosporus (also known as Brevibacillus laterosporus), Bacillus lautus, Bacillus lentimorbus, Bacillus lentus, Bacillus licheniformis, Bacillus maroccanus, Bacillus megaterium, Bacillus metiens, Bacillus mycoides, Bacillus natto, Bacillus nematocida, Bacillus nigrificans, Bacillus nigrum, Bacillus pantothenticus, Bacillus popillae, Bacillus psychrosaccharolyticus, Bacillus pumilus, Bacillus siamensis, Bacillus smithii, Bacillus sphaericus, Bacillus subtilis, Bacillus thuringiensis, Bacillus uniflagellatus, Bradyrhizobium japonicum, Brevibacillus brevis, Brevibacillus laterosporus (formerly Bacillus laterosporus), Chromobacterium subtsugae, Delftia acidovorans, Lactobacillus acidophilus, Lysobacter antibioticus, Lysobacter enzymogenes, Paenibacillus alvei, Paenibacillus polymyxa, Paenibacillus popilliae (formerly Bacillus popilliae), Pantoea agglomerans, Pasteuria penetrans (formerly Bacillus penetrans), Pasteuria usgae, Pectobacterium carotovorum (formerly Erwinia carotovora), Pseudomonas aeruginosa, Pseudomonas aureofaciens, Pseudomonas cepacia (formerly known as Burkholderia cepacia), Pseudomonas chlororaphis, Pseudomonas fluorescens, Pseudomonas proradix, Pseudomonas putida, Pseudomonas syringae, Serratia entomophila, Serratia marcescens, Streptomyces colombiensis, Streptomyces galbus, Streptomyces goshikiensis, Streptomyces griseoviridis, Streptomyces lavendulae, Streptomyces prasinus, Streptomyces saraceticus, Streptomyces venezuelae, Xanthomonas campestris, Xenorhabdus luminescens, Xenorhabdus nematophila, Rhodococcus globerulus AQ719 (NRRL Accession No. B-21663), Bacillus sp. AQ175 (ATCC Accession No. 55608), Bacillus sp. AQ 177 (ATCC Accession No. 55609), Bacillus sp. AQ178 (ATCC Accession No. 53522), and Streptomyces sp. strain NRRL Accession No. B-30145. In some embodiments, the bacterium is Azotobacter chroococcum, Methanosarcina barkeri, Klesiella pneumoniae, Azotobacter vinelandii, Rhodobacter spharoides, Rhodobacter capsulatus, Rhodobcter palustris, Rhodosporillum rubrum, Rhizobium leguminosarum or Rhizobium etli.

In some embodiments, the engineered microbes used with the compositions, methods, kits, and systems of the present disclosure is from the genus Clostridium, for example Clostridium pasteurianum, Clostridium beijerinckii, Clostridium perfringens, Clostridium tetani, Clostridium acetobutylicum.

In some embodiments, the engineered microbes used with the compositions, methods, kits, and systems of the present disclosure are cyanobacteria. Examples of cyanobacterial genera include Anabaena (for example Anagaena sp. PCC7120), Nostoc (for example Nostoc punctiforme), or Synechocystis (for example Synechocystis sp. PCC6803).

In some embodiments, the engineered microbes used with the compositions, methods, kits, and systems of the present disclosure belong to the phylum Chlorobi, for example Chlorobium tepidum.

In some embodiments, the engineered microbes used with the compositions, methods, kits, and systems of the present disclosure comprise a gene homologous to a known NifH gene. Sequences of known NifH genes may be found in, for example, the Zehr lab NifH database, (wwwzehr.pmc.ucsc.edu/nifH_Database_Public/, Apr. 4, 2014), or the Buckley lab NifH database (www.css.cornell.edu/faculty/buckley/nifh.htm, and Gaby, John Christian, and Daniel H. Buckley. “A comprehensive aligned nifH gene database: a multipurpose tool for studies of nitrogen-fixing bacteria.” Database 2014 (2014): bau001.). In some aspects, the engineered microbes used with the compositions, methods, kits, and systems of the present disclosure comprise a sequence which encodes a polypeptide with at least 60%, 70%, 80%, 85%, 90%, 95%, 96%, 96%, 98%, 99% or more than 99% sequence identity to a sequence from the Zehr lab NifH database, (wwwzehr.pmc.ucsc.edu/nifH_Database_Public/, Apr. 4, 2014). In some aspects, the engineered microbes used with the compositions, methods, kits, and systems of the present disclosure comprise a sequence which encodes a polypeptide with at least 60%, 70%, 80%, 85%, 90%, 95%, 96%, 96%, 98%, 99% or more than 99% sequence identity to a sequence from the Buckley lab NifH database, (Gaby, John Christian, and Daniel H. Buckley. “A comprehensive aligned nifH gene database: a multipurpose tool for studies of nitrogen-fixing bacteria.” Database 2014 (2014): bau001.).

In some embodiments, the compositions, methods, kits, and systems described herein make use of bacteria that are able to self-propagate efficiently on the leaf surface, root surface, or inside plant tissues without inducing a damaging plant defense reaction, or bacteria that are resistant to plant defense responses. In some embodiments, the bacteria described herein are isolated by culturing a plant tissue extract or leaf surface wash in a medium with no added nitrogen.

In some embodiments, the engineered microbe is an endophyte or an epiphyte or a bacterium inhabiting the plant rhizosphere (rhizospheric bacteria). Endophytes are organisms that enter the interior of plants without causing disease symptoms or eliciting the formation of symbiotic structures, and are of agronomic interest because they can enhance plant growth and improve the nutrition of plants (e.g., through nitrogen fixation). The bacteria can be a seed-borne endophyte. Seed-borne endophytes include bacteria associated with or derived from the seed of a grass or plant, such as a seed-borne bacterial endophyte found in mature, dry, undamaged (e.g., no cracks, visible fungal infection, or prematurely germinated) seeds. The seed-borne bacterial endophyte can be associated with or derived from the surface of the seed; alternatively, or in addition, it can be associated with or derived from the interior seed compartment (e.g., of a surface-sterilized seed). In some aspects, a seed-borne bacterial endophyte is capable of replicating within the plant tissue, for example, the interior of the seed. Also, In some aspects, the seed-borne bacterial endophyte is capable of surviving desiccation.

Further, one skilled in the art will understand that assemblages of microbes, for example those that exhibit complementary colonization (different nutrient utilization, temporal occupation, oxygen adaptability, and/or spatial occupation), and/or different benefits to the seed or plant (nitrogen fixation, pest and/or pathogen control, etc.) can be used with the compositions, methods, kits, and systems disclosed herein to increase on-seed adherence and stability of the microbe(s).

Thus, the engineered microbes used in the compositions, methods, kits, and systems of the disclosure, can comprise a plurality of different microorganism taxa in combination. By way of example, the bacteria may include Proteobacteria (such as Pseudomonas, Enterobacter, Stenotrophomonas, Burkholderia, Rhizobium, Herbaspirillum, Pantoea, Serratia, Rahnella, Azospirillum, Azorhizobium, Azotobacter, Duganella, Delftia, Bradyrhizobiun, Sinorhizobium and Halomonas), Firmicutes (such as Bacillus, Paenibacillus, Lactobacillus, Mycoplasma, and Acetobacterium), and Actinobacteria (such as Streptomyces, Rhodacoccus, Microbacterium, and Curtobacterium). The bacteria used in the compositions, methods, kits, and systems of this disclosure may include nitrogen fixing bacterial consortia of two or more species. In some embodiments, one or more bacterial species of the bacterial consortia may be capable of fixing nitrogen. In some embodiments, one or more species of the bacterial consortia facilitate or enhance the ability of other bacteria to fix nitrogen. The bacteria which fix nitrogen and the bacteria which enhance the ability of other bacteria to fix nitrogen may be the same or different. In some aspects, a bacterial strain is able to fix nitrogen when in combination with a different bacterial strain, or in a certain bacterial consortia, but may be unable to fix nitrogen in a monoculture. Examples of bacterial genera which may be found in a nitrogen fixing bacterial consortia include, but are not limited to, Herbaspirillum, Azospirillum, Enterobacter, and Bacillus.

Engineered bacteria that can be used in the compositions and methods disclosed herein include Azotobacter sp., Bradyrhizobium sp., Klebsiella sp., and Sinorhizobium sp. In some aspects, the bacteria are selected from the group consisting of: Azotobacter vinelandii, Bradyrhizobium japonicum, Klebsiella pneumoniae, and Sinorhizobium meliloti. In some aspects, the bacteria are of the genus Enterobacter or Rahnella. In some aspects, the bacteria are of the genus Frankia, or Clostridium. Examples of bacteria of the genus Clostridium include, but are not limited to, Clostridium acetobutylicum, Clostridium pasteurianum, Clostridium beijerinckii, Clostridium perfringens, and Clostridium tetani. In some aspects, the bacteria are of the genus Paenibacillus, for example Paenibacillus azotofixans, Paenibacillus borealis, Paenibacillus durus, Paenibacillus macerans, Paenibacillus polymyxa, Paenibacillus alvei, Paenibacillus amylolyticus, Paenibacillus campinasensis, Paenibacillus chibensis, Paenibacillus glucanolyticus, Paenibacillus illinoisensis, Paenibacillus larvae subsp. Larvae, Paenibacillus larvae subsp. Pulvifaciens, Paenibacillus lautus, Paenibacillus macerans, Paenibacillus macquariensis, Paenibacillus macquariensis, Paenibacillus pabuli, Paenibacillus peoriae, or Paenibacillus polymyxa.

In some embodiments, the engineered bacteria for use in the present compositions and methods can be a member of one or more of the following taxa: Achromobacter, Acidithiobacillus, Acidovorax, Acidovoraz, Acinetobacter, Actinoplanes, Adlercreutzia, Aerococcus, Aeromonas, Afipia, Agromyces, Ancylobacter, Arthrobacter, Atopostipes, Azospirillum, Bacillus, Bdellovibrio, Beijerinckia, Bosea, Bradyrhizobium, Brevibacillus, Brevundimonas, Burkholderia, Candidatus Haloredivivus, Caulobacter, Cellulomonas, Cellvibrio, Chryseobacterium, Citrobacter, Clostridium, Coraliomargarita, Corynebacterium, Cupriavidus, Curtobacterium, Curvibacter, Deinococcus, Delftia, Desemzia, Devosia, Dokdonella, Dyella, Enhydrobacter, Enterobacter, Enterococcus, Erwinia, Escherichia, Escherichia/Shigella, Exiguobacterium, Ferroglobus, Filimonas, Finegoldia, Flavisolibacter, Flavobacterium, Frigoribacterium, Gluconacetobacter, Hafnia, Halobaculum, Halomonas, Halosimplex, Herbaspirillum, Hymenobacter, Klebsiella, Kocuria, Kosakonia, Lactobacillus, Leclercia, Lentzea, Luteibacter, Luteimonas, Massilia, Mesorhizobium, Methylobacterium, Microbacterium, Micrococcus, Microvirga, Mycobacterium, Neisseria, Nocardia, Oceanibaculum, Ochrobactrum, Okibacterium, Oligotropha, Oryzihumus, Oxalophagus, Paenibacillus, Panteoa, Pantoea, Pelomonas, Perlucidibaca, Plantibacter, Polynucleobacter, Propionibacterium, Propioniciclava, Pseudoclavibacter, Pseudomonas, Pseudonocardia, Pseudoxanthomonas, Psychrobacter, Rahnella, Ralstonia, Rheinheimera, Rhizobium, Rhodococcus, Rhodopseudomonas, Roseateles, Ruminococcus, Sebaldella, Sediminibacillus, Sediminibacterium, Serratia, Shigella, Shinella, Sinorhizobium, Sinosporangium, Sphingobacterium, Sphingomonas, Sphingopyxis, Sphingosinicella, Staphylococcus, Stenotrophomonas, Strenotrophomonas, Streptococcus, Streptomyces, Stygiolobus, Sulfurisphaera, Tatumella, Tepidimonas, Thermomonas, Thiobacillus, Variovorax, WPS-2 genera incertae sedis, Xanthomonas, and Zimmermannella.

In some embodiments, the engineered microbes are Gram-negative bacteria of a genus selected from the following list: Acetobacter, Achromobacter, Aerobacter, Anabaena, Azoarcus, Azomonas, Azorhizobium, Azospirillum, Azotobacter, Beijernickia, Bradyrhizobium, Burkholderia, Citrobacter, Derxia, Enterobacter, Herbaspirillum, Klebsiella, Kluyvera, Kosakonia, Nostoc, Mesorhizobium, Rahnella, Rhizobium, Rhodobacter, Rhodopseudomonas, Rhodospirillum, Serratia Sinorhizobium, Spirillum, Trichodesmium, and Xanthomonas.

In some embodiments, a bacterial species selected from at least one of the following genera are utilized: Enterobacter, Klebsiella, Kosakonia, and Rahnella. In some aspects, a combination of bacterial species from the following genera are utilized: Enterobacter, Klebsiella, Kosakonia, and Rahnella. In some aspects, the species utilized can be one or more of: Enterobacter sacchari, Klebsiella variicola, Kosakonia sacchari, and Rahnella aquatilis.

In some embodiments, the engineered microbe is a Gram positive microbe having a Molybdenum-Iron nitrogenase system comprising: nifH, nifD, nifK, nifB, nifE, nifN, nifX, hesA, nifV, nifW, nifU, nifS, nifI1, and nifI2. In some aspects, a Gram positive microbe may have a vanadium nitrogenase system comprising: vnfDG, vnfK, vnfE, vnfN, vupC, vupB, vupA, vnfV, vnfR1, vnfH, vnfR2, vnfA (transcriptional regulator). In some aspects, a Gram positive microbe may have an iron-only nitrogenase system comprising: anfK, anfG, anfD, anfH, anfA (transcriptional regulator). In some aspects, a Gram positive microbe may have a nitrogenase system comprising glnB, and glnK (nitrogen signaling proteins). Some examples of enzymes involved in nitrogen metabolism in Gram positive microbes include glnA (glutamine synthetase), gdh (glutamate dehydrogenase), bdh (3-hydroxybutyrate dehydrogenase), glutaminase, gltAB/gltB/gltS (glutamate synthase), asnA/asnB (aspartate-ammonia ligase/asparagine synthetase), and ansA/ansZ (asparaginase). Some examples of proteins involved in nitrogen transport in Gram positive microbes include amtB (ammonium transporter), glnK (regulator of ammonium transport), glnPHQ/glnQHMP (ATP-dependent glutamine/glutamate transporters), glnT/alsT/yrbD/yflA (glutamine-like proton symport transporters), and gltP/gltT/yhcl/nqt (glutamate-like proton symport transporters).

Examples of engineered Gram positive microbes for use within the present compositions include Paenibacillus polymixa, Paenibacillus riograndensis, Paenibacillus sp., Frankia sp., Heliobacterium sp., Heliobacterium chlorum, Heliobacillus sp., Heliophilum sp., Heliorestis sp., Clostridium acetobutylicum, Clostridium sp., Methanobacterium sp., Micrococcus sp., Mycobacterium flavum, Mycobacterium sp., Arthrobacter sp., Agromyces sp., Corynebacterium autitrophicum, Corynebacterium sp., Micromonospora sp., Propionibacteria sp., Streptomyces sp., and Microbacterium sp.

### Genetic Alterations to Microbes—Methods

In some embodiments, the microorganism which is combined with the compositions disclosed herein is genetically engineered to have improved nitrogen fixation capabilities. Thus, in some aspects, the microbes comprise one or more genetic variations introduced into one or more genes regulating nitrogen fixation. In some aspects, the genetic variation is introduced by targeted mutagenesis, insertion, or deletion of nucleic acid. In some aspects, the genetic variation is introduced via induced mutagenesis. In some aspects, the genetic variation is introduced via spontaneous mutation. In some aspects, the mutation is introduced without the use of an artificial means for inducing mutations (e.g., a mutagen known in the art and/or provided herein) in the genomic and extragenomic DNA such that the DNA of the microbes accumulate one or more mutations.

In general, the term “genetic variation” refers to any change introduced into a polynucleotide sequence relative to a reference polynucleotide, such as a reference genome or portion thereof, or reference gene or portion thereof. A genetic variation may be referred to as a “mutation,” and a sequence or organism comprising a genetic variation may be referred to as a “genetic variant” or “mutant”. Genetic variations can have any number of effects, such as the increase or decrease of some biological activity, including gene expression, metabolism, and cell signaling. Genetic variations can be specifically introduced to a target site, or introduced randomly. A variety of molecular tools and methods are available for introducing genetic variation. For example, genetic variation can be introduced via polymerase chain reaction mutagenesis, oligonucleotide-directed mutagenesis, saturation mutagenesis, fragment shuffling mutagenesis, homologous recombination, recombineering, lambda red mediated recombination, CRISPR/Cas9 systems, chemical mutagenesis, and combinations thereof. Chemical methods of introducing genetic variation include exposure of DNA to a chemical mutagen, e.g., ethyl methanesulfonate (EMS), methyl methanesulfonate (MMS), N-nitrosourea (EN U), N-methyl-N-nitro-N′-nitrosoguanidine, 4-nitroquinoline N-oxide, diethylsulfate, benzopyrene, cyclophosphamide, bleomycin, triethylmelamine, acrylamide monomer, nitrogen mustard, vincristine, diepoxyalkanes (for example, diepoxybutane), ICR-170, formaldehyde, procarbazine hydrochloride, ethylene oxide, dimethylnitrosamine, 7,12 dimethylbenz (a) anthracene, chlorambucil, hexamethylphosphoramide, bisulfan, and the like. Radiation mutation-inducing agents include ultraviolet radiation, γ-irradiation, X-rays, and fast neutron bombardment. Genetic variation can also be introduced into a nucleic acid using, e.g., trimethylpsoralen with ultraviolet light. Random or targeted insertion of a mobile DNA element, e.g., a transposable element, is another suitable method for generating genetic variation. Genetic variations can be introduced into a nucleic acid during amplification in a cell-free in vitro system, e.g., using a polymerase chain reaction (PCR) technique such as error-prone PCR. Genetic variations can be introduced into a nucleic acid in vitro using DNA shuffling techniques (e.g., exon shuffling, domain swapping, and the like). Genetic variations can also be introduced into a nucleic acid as a result of a deficiency in a DNA repair enzyme in a cell, e.g., the presence in a cell of a mutant gene encoding a mutant DNA repair enzyme is expected to generate a high frequency of mutations (i.e., about 1 mutation/100 genes-1 mutation/10,000 genes) in the genome of the cell. Examples of genes encoding DNA repair enzymes include but are not limited to Mut H, Mut S, Mut L, and Mut U, and the homologs thereof in other species (e.g., MSH 1 6, PMS 1 2, MLH 1, GTBP, ERCC-1, and the like). Example descriptions of various methods for introducing genetic variations are provided in e.g., Stemple (2004) Nature 5:1-7; Chiang et al. (1993) PCR Methods Appl 2 (3): 210-217; Stemmer (1994) Proc. Natl. Acad. Sci. USA 91:10747-10751; and U.S. Pat. Nos. 6,033,861, and 6,773,900.

Genetic variations introduced into microbes may be classified as transgenic, cisgenic, intragenomic, intrageneric, intergeneric, synthetic, evolved, rearranged, or SNPs.

Genetic variation may be introduced into numerous metabolic pathways within microbes to elicit improvements in the traits described herein. Representative pathways include sulfur uptake pathways, glycogen biosynthesis, the glutamine regulation pathway, the molybdenum uptake pathway, the nitrogen fixation pathway, ammonia assimilation, ammonia excretion or secretion, nNitrogen uptake, glutamine biosynthesis, annamox, phosphate solubilization, organic acid transport, organic acid production, agglutinins production, reactive oxygen radical scavenging genes, Indole Acetic Acid biosynthesis, trehalose biosynthesis, plant cell wall degrading enzymes or pathways, root attachment genes, exopolysaccharide secretion, glutamate synthase pathway, iron uptake pathways, siderophore pathway, chitinase pathway, ACC deaminase, glutathione biosynthesis, phosphorous signalig genes, quorum quenching pathway, cytochrome pathways, hemoglobin pathway, bacterial hemoglobin-like pathway, small RNA rsmZ, rhizobitoxine biosynthesis, lapA adhesion protein, AHL quorum sensing pathway, phenazine biosynthesis, cyclic lipopeptide biosynthesis, and antibiotic production.

CRISPR/Cas9 (Clustered regularly interspaced short palindromic repeats)/CRISPR-associated (Cas) systems can be used to introduce desired mutations. CRISPR/Cas9 provide bacteria and archaea with adaptive immunity against viruses and plasmids by using CRISPR RNAs (crRNAs) to guide the silencing of invading nucleic acids. The Cas9 protein (or functional equivalent and/or variant thereof, i.e., Cas9-like protein) naturally contains DNA endonuclease activity that depends on the association of the protein with two naturally occurring or synthetic RNA molecules called crRNA and tracrRNA (also called guide RNAs). In some cases, the two molecules are covalently link to form a single molecule (also called a single guide RNA (“sgRNA”). Thus, the Cas9 or Cas9-like protein associates with a DNA-targeting RNA (which term encompasses both the two-molecule guide RNA configuration and the single-molecule guide RNA configuration), which activates the Cas9 or Cas9-like protein and guides the protein to a target nucleic acid sequence. If the Cas9 or Cas9-like protein retains its natural enzymatic function, it will cleave target DNA to create a double-stranded break, which can lead to genome alteration (i.e., editing: deletion, insertion (when a donor polynucleotide is present), replacement, etc.), thereby altering gene expression. Some variants of Cas9 (which variants are encompassed by the term Cas9-like) have been altered such that they have a decreased DNA cleaving activity (in some cases, they cleave a single strand instead of both strands of the target DNA, while in other cases, they have severely reduced to no DNA cleavage activity). Further exemplary descriptions of CRISPR systems for introducing genetic variation can be found in, e.g. U.S. Pat. No. 8,795,965.

As a cyclic amplification technique, polymerase chain reaction (PCR) mutagenesis uses mutagenic primers to introduce desired mutations. PCR is performed by cycles of denaturation, annealing, and extension. After amplification by PCR, selection of mutated DNA and removal of parental plasmid DNA can be accomplished by: 1) replacement of dCTP by hydroxymethylated-dCTP during PCR, followed by digestion with restriction enzymes to remove non-hydroxymethylated parent DNA only; 2) simultaneous mutagenesis of both an antibiotic resistance gene and the studied gene changing the plasmid to a different antibiotic resistance, the new antibiotic resistance facilitating the selection of the desired mutation thereafter; 3) after introducing a desired mutation, digestion of the parent methylated template DNA by restriction enzyme Dpnl which cleaves only methylated DNA, by which the mutagenized unmethylated chains are recovered; or 4) circularization of the mutated PCR products in an additional ligation reaction to increase the transformation efficiency of mutated DNA. Further description of exemplary methods can be found in e.g. U.S. Pat. Nos. 7,132,265, 6,713,285, 6,673,610, 6,391,548, 5,789,166, 5,780,270, 5,354,670, 5,071,743, and US20100267147.

Oligonucleotide-directed mutagenesis, also called site-directed mutagenesis, typically utilizes a synthetic DNA primer. This synthetic primer contains the desired mutation and is complementary to the template DNA around the mutation site so that it can hybridize with the DNA in the gene of interest. The mutation may be a single base change (a point mutation), multiple base changes, deletion, or insertion, or a combination of these. The single-strand primer is then extended using a DNA polymerase, which copies the rest of the gene. The gene thus copied contains the mutated site, and may then be introduced into a host cell as a vector and cloned. Finally, mutants can be selected by DNA sequencing to check that they contain the desired mutation.

Genetic variations can be introduced using error-prone PCR. In this technique the gene of interest is amplified using a DNA polymerase under conditions that are deficient in the fidelity of replication of sequence. The result is that the amplification products contain at least one error in the sequence. When a gene is amplified and the resulting product(s) of the reaction contain one or more alterations in sequence when compared to the template molecule, the resulting products are mutagenized as compared to the template. Another means of introducing random mutations is exposing cells to a chemical mutagen, such as nitrosoguanidine or ethyl methanesulfonate (Nestmann, Mutat Res 1975 June; 28 (3): 323-30), and the vector containing the gene is then isolated from the host.

Saturation mutagenesis is another form of random mutagenesis, in which one tries to generate all or nearly all possible mutations at a specific site, or narrow region of a gene. In a general sense, saturation mutagenesis is comprised of mutagenizing a complete set of mutagenic cassettes (wherein each cassette is, for example, 1-500 bases in length) in defined polynucleotide sequence to be mutagenized (wherein the sequence to be mutagenized is, for example, from 15 to 100, 000 bases in length). Therefore, a group of mutations (e.g. ranging from 1 to 100 mutations) is introduced into each cassette to be mutagenized. A grouping of mutations to be introduced into one cassette can be different or the same from a second grouping of mutations to be introduced into a second cassette during the application of one round of saturation mutagenesis. Such groupings are exemplified by deletions, additions, groupings of particular codons, and groupings of particular nucleotide cassettes.

Fragment shuffling mutagenesis, also called DNA shuffling, is a way to rapidly propagate beneficial mutations. In an example of a shuffling process, DNAse is used to fragment a set of parent genes into pieces of e.g. about 50-100 bp in length. This is then followed by a polymerase chain reaction (PCR) without primers—DNA fragments with sufficient overlapping homologous sequence will anneal to each other and are then be extended by DNA polymerase. Several rounds of this PCR extension are allowed to occur, after some of the DNA molecules reach the size of the parental genes. These genes can then be amplified with another PCR, this time with the addition of primers that are designed to complement the ends of the strands. The primers may have additional sequences added to their 5′ ends, such as sequences for restriction enzyme recognition sites needed for ligation into a cloning vector. Further examples of shuffling techniques are provided in US20050266541.

Homologous recombination mutagenesis involves recombination between an exogenous DNA fragment and the targeted polynucleotide sequence. After a double-stranded break occurs, sections of DNA around the 5′ ends of the break are cut away in a process called resection. In the strand invasion step that follows, an overhanging 3′ end of the broken DNA molecule then “invades” a similar or identical DNA molecule that is not broken. The method can be used to delete a gene, remove exons, add a gene, and introduce point mutations. Homologous recombination mutagenesis can be permanent or conditional. Typically, a recombination template is also provided. A recombination template may be a component of another vector, contained in a separate vector, or provided as a separate polynucleotide. In some embodiments, a recombination template is designed to serve as a template in homologous recombination, such as within or near a target sequence nicked or cleaved by a site-specific nuclease. A template polynucleotide may be of any suitable length, such as about or more than about 10, 15, 20, 25, 50, 75, 100, 150, 200, 500, 1000, or more nucleotides in length. In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence. Non-limiting examples of site-directed nucleases useful in methods of homologous recombination include zinc finger nucleases, CRISPR nucleases, TALE nucleases, and meganuclease. For a further description of the use of such nucleases, see e.g. U.S. Pat. No. 8,795,965 and US20140301990.

Mutagens that create primarily point mutations and short deletions, insertions, transversions, and/or transitions, including chemical mutagens or radiation, may be used to create genetic variations. Mutagens include, but are not limited to, ethyl methanesulfonate, methylmethane sulfonate, N-ethyl-N-nitrosurea, triethylmelamine, N-methyl-N-nitrosourea, procarbazine, chlorambucil, cyclophosphamide, diethyl sulfate, acrylamide monomer, melphalan, nitrogen mustard, vincristine, dimethylnitrosamine, N-methyl-N′-nitro-Nitrosoguanidine, nitrosoguanidine, 2-aminopurine, 7,12 dimethyl-benz (a) anthracene, ethylene oxide, hexamethylphosphoramide, bisulfan, diepoxyalkanes (diepoxyoctane, diepoxybutane, and the like), 2-methoxy-6-chloro-9 [3-(ethyl-2-chloro-ethyl)aminopropylamino]acridine dihydrochloride and formaldehyde.

Introducing genetic variation may be an incomplete process, such that some bacteria in a treated population of bacteria carry a desired mutation while others do not. In some cases, it is desirable to apply a selection pressure so as to enrich for bacteria carrying a desired genetic variation. Traditionally, selection for successful genetic variants involved selection for or against some functionality imparted or abolished by the genetic variation, such as in the case of inserting antibiotic resistance gene or abolishing a metabolic activity capable of converting a non-lethal compound into a lethal metabolite. It is also possible to apply a selection pressure based on a polynucleotide sequence itself, such that only a desired genetic variation need be introduced (e.g. without also requiring a selectable marker). In this case, the selection pressure can comprise cleaving genomes lacking the genetic variation introduced to a target site, such that selection is effectively directed against the reference sequence into which the genetic variation is sought to be introduced. Typically, cleavage occurs within 100 nucleotides of the target site (e.g. within 75, 50, 25, 10, or fewer nucleotides from the target site, including cleavage at or within the target site). Cleaving may be directed by a site-specific nuclease selected from the group consisting of a Zinc Finger nuclease, a CRISPR nuclease, a TALE nuclease (TALEN), or a meganuclease. Such a process is similar to processes for enhancing homologous recombination at a target site, except that no template for homologous recombination is provided. As a result, bacteria lacking the desired genetic variation are more likely to undergo cleavage that left unrepaired, results in cell death. Bacteria surviving selection may then be isolated for use in exposing to plants for assessing conferral of an improved trait.

A CRISPR nuclease may be used as the site-specific nuclease to direct cleavage to a target site. An improved selection of mutated microbes can be obtained by using Cas9 to kill non-mutated cells. Plants are then inoculated with the mutated microbes to re-confirm symbiosis and create evolutionary pressure to select for efficient symbionts. Microbes can then be re-isolated from plant tissues. CRISPR nuclease systems employed for selection against non-variants can employ similar elements to those described above with respect to introducing genetic variation, except that no template for homologous recombination is provided. Cleavage directed to the target site thus enhances death of affected cells.

Other options for specifically inducing cleavage at a target site are available, such as zinc finger nucleases, TALE nuclease (TALEN) systems, and meganuclease. Zinc-finger nucleases (ZFNs) are artificial DNA endonucleases generated by fusing a zinc finger DNA binding domain to a DNA cleavage domain. ZFNs can be engineered to target desired DNA sequences and this enables zinc-finger nucleases to cleave unique target sequences. When introduced into a cell, ZFNs can be used to edit target DNA in the cell (e.g., the cell's genome) by inducing double stranded breaks. Transcription activator-like effector nucleases (TALENs) are artificial DNA endonucleases generated by fusing a TAL (Transcription activator-like) effector DNA binding domain to a DNA cleavage domain. TALENS can be quickly engineered to bind practically any desired DNA sequence and when introduced into a cell, TALENs can be used to edit target DNA in the cell (e.g., the cell's genome) by inducing double strand breaks. Meganucleases (homing endonuclease) are endodeoxyribonucleases characterized by a large recognition site (double-stranded DNA sequences of 12 to 40 base pairs. Meganucleases can be used to replace, eliminate or modify sequences in a highly targeted way. By modifying their recognition sequence through protein engineering, the targeted sequence can be changed. Meganucleases can be used to modify all genome types, whether bacterial, plant or animal and are commonly grouped into four families: the LAGLIDADG family, the GIY-YIG family, the His-Cyst box family and the HNH family. Exemplary homing endonucleases include I-Scel, I-CeuI, PI-PspI, PI-Sce, I-SceIV, I-CsmI, I-PanI, I-Scell, I-PpoI, I-SceIII, I-Crel, I-TevI, I-TevII and I-TevIII.

### Genetic Alterations to Microbes—Locations and Sources of Genetic Variation

The genetic variation may be introduced into a gene selected from the group consisting of nifA, nifL, ntrB, ntrC, glutamine synthetase, glnA, glnB, glnK, draT, amtB, glutaminase, glnD, glnE, nifJ, nifH, nifD, nifK, nifY, nifE, nifN, nifU, nifS, nifV, nifW, nifZ, nifM, nifF, nifB, and nifQ. The genetic variation may be a variation in a gene encoding a protein with functionality selected from the group consisting of: glutamine synthetase, glutaminase, glutamine synthetase adenylyltransferase, transcriptional activator, anti-transcriptional activator, pyruvate flavodoxin oxidoreductase, flavodoxin, and NAD+-dinitrogen-reductase aDP-D-ribosyltransferase. The genetic variation may be a mutation that results in one or more of: increased expression or activity of nifA or glutaminase; decreased expression or activity of nifL, ntrB, glutamine synthetase, glnB, glnK, draT, amtB; decreased adenylyl-removing activity of GlnE; decreased expression of GlnD; or decreased uridylyl-removing activity of GlnD. The genetic variation may be a variation in a gene selected from the group consisting of: bcsii, bcsiii, yjbE, fhaB, pehA, otsB, treZ, glsA2, and combinations thereof.

In some embodiments, the microbe has a disrupted (e.g., deleted or partially deleted) nifL gene. In some aspects, the microbe has a nifL gene that has been disrupted with the introduction of a promoter sequence that acts on the nifA gene. In some aspects, e.g., when the microbe is a strain of K. variicola, the promoter is a K. variicola PinfC promoter. In some aspects, e.g., when the microbe is a strain of K. sacchari, the promoter is a K. sacchari Prm5 promoter. In some aspects, the microbe has a glnE gene that has been altered to remove the adenylyl-removing (AR) domain, while leaving the coding region for the adenyltransferase (AT) domain, which is functionally expressed. In some aspects, the microbe has a deletion of the glnD gene.

The genetic variation introduced into one or more microorganisms may be a knock-out mutation or it may abolish a regulatory sequence of a target gene, or it may comprise insertion of a heterologous regulatory sequence, for example, insertion of a regulatory sequence found within the genome of the same bacterial species or genus. The regulatory sequence can be chosen based on the expression level of a gene in a bacterial culture or within plant tissue. The genetic variation may be produced by chemical mutagenesis. The plants grown may be exposed to biotic or abiotic stressors. However, in some aspects, the one or more cultured microbes for use with the compositions and methods disclosed herein also envision altering the impact of ATP or O₂ on the circuitry, or replacing the circuitry with other regulatory cascades in the cell, or altering genetic circuits other than nitrogen fixation. Gene clusters can be re-engineered to generate functional products under the control of a heterologous regulatory system. By eliminating native regulatory elements outside of, and within, coding sequences of gene clusters, and replacing them with alternative regulatory systems, the functional products of complex genetic operons and other gene clusters can be controlled and/or moved to heterologous cells, including cells of different species other than the species from which the native genes were derived. Once re-engineered, the synthetic gene clusters can be controlled by genetic circuits or other inducible regulatory systems, thereby controlling the products' expression as desired. The expression cassettes can be designed to act as logic gates, pulse generators, oscillators, switches, or memory devices. The controlling expression cassette can be linked to a promoter such that the expression cassette functions as an environmental sensor, such as an oxygen, temperature, touch, osmotic stress, membrane stress, or redox sensor.

As an example, the nifL, nifA, nifT, and nifX genes can be eliminated from the nif gene cluster. Synthetic genes can be designed by codon randomizing the DNA encoding each amino acid sequence. Codon selection is performed, specifying that codon usage be as divergent as possible from the codon usage in the native gene. Proposed sequences are scanned for any undesired features, such as restriction enzyme recognition sites, transposon recognition sites, repetitive sequences, sigma 54 and sigma 70 promoters, cryptic ribosome binding sites, and rho independent terminators. Synthetic ribosome binding sites are chosen to match the strength of each corresponding native ribosome binding site, such as by constructing a fluorescent reporter plasmid in which the 150 bp surrounding a gene's start codon (from-60 to +90) is fused to a fluorescent gene. This chimera can be expressed under control of the Ptac promoter, and fluorescence measured via flow cytometry. To generate synthetic ribosome binding sites, a library of reporter plasmids using 150 bp (−60 to +90) of a synthetic expression cassette is generated. Briefly, a synthetic expression cassette can consist of a random DNA spacer, a degenerate sequence encoding an RBS library, and the coding sequence for each synthetic gene. Multiple clones are screened to identify the synthetic ribosome binding site that best matched the native ribosome binding site. Synthetic operons that consist of the same genes as the native operons are thus constructed and tested for functional complementation. A further exemplary description of synthetic operons is provided in US20140329326.

Some examples of genetic alterations which may be made in Gram positive microbes include: deleting glnR to remove negative regulation of BNF in the presence of environmental nitrogen, inserting different promoters directly upstream of the nif cluster to eliminate regulation by GlnR in response to environmental nitrogen, mutating glnA to reduce the rate of ammonium assimilation by the GS-GOGAT pathway, deleting amtB to reduce uptake of ammonium from the media, mutating glnA so it is constitutively in the feedback-inhibited (FBI-GS) state, to reduce ammonium assimilation by the GS-GOGAT pathway.

GlnR is the main regulator of N metabolism and fixation in, e.g., Paenibacillus species. In some aspects, the genome of a Paenibacillus species does not contain a gene to produce glnR. In some aspects, the genome of a Paenibacillus species does not contain a gene to produce glnE or glnD. In some aspects, the genome of a Paenibacillus species does contain a gene to produce glnB or glnK. For example, Paenibacillus sp. WLY78 doesn't contain a gene for glnB, or its homologs found in the archaeon Methanococcus maripaludis, nifI1 and nif12. In some aspects, the genomes of Paenibacillus species are variable. For example, Paenibacillus polymixa E681 lacks glnK and gdh, has several nitrogen compound transporters, but only amtB appears to be controlled by GlnR. In another example, Paenibacillus sp. JDR2 has glnK, gdh and most other central nitrogen metabolism genes, has many fewer nitrogen compound transporters, but does have glnPHQ controlled by GlnR. Paenibacillus riograndensis SBR5 contains a standard glnRA operon, an fdx gene, a main nif operon, a secondary nif operon, and an anf operon (encoding iron-only nitrogenase). Putative glnR/tnrA sites were found upstream of each of these operons. GlnR may regulate all of the above operons, except the anf operon. GlnR may bind to each of these regulatory sequences as a dimer.

Paenibacillus N-fixing strains may fall into two subgroups: Subgroup I, which contains only a minimal nif gene cluster and subgroup II, which contains a minimal cluster, plus an uncharacterized gene between nifX and hesA, and often other clusters duplicating some of the nif genes, such as nifH, nifHDK, nifBEN, or clusters encoding vanadaium nitrogenase (vnf) or iron-only nitrogenase (anf) genes.

In some embodiments, the genome of a Paenibacillus species may not contain a gene to produce glnB or glnK. In some aspects, the genome of a Paenibacillus species may contain a minimal nif cluster with 9 genes transcribed from a sigma-70 promoter. In some aspects, a Paenibacillus nif cluster is negatively regulated by nitrogen or oxygen. In some aspects, the genome of a Paenibacillus species does not contain a gene to produce sigma-54. For example, Paenibacillus sp. WLY78 does not contain a gene for sigma-54. In some aspects, a nif cluster is regulated by glnR, and/or TnrA. In some aspects, activity of a nif cluster is altered by altering activity of glnR, and/or TnrA.

In Bacilli, glutamine synthetase (GS) is feedback-inhibited by high concentrations of intracellular glutamine, causing a shift in confirmation (referred to as FBI-GS). Nif clusters contain distinct binding sites for the regulators GlnR and TnrA in several Bacilli species. GlnR binds and represses gene expression in the presence of excess intracellular glutamine and AMP. A role of GlnR may be to prevent the influx and intracellular production of glutamine and ammonium under conditions of high nitrogen availability. TnrA may bind and/or activate (or repress) gene expression in the presence of limiting intracellular glutamine, and/or in the presence of FBI-GS. In some embodiments, the activity of a Bacilli nif cluster is altered by altering the activity of GlnR.

Feedback-inhibited glutamine synthetase (FBI-GS) may bind GlnR and stabilize binding of GlnR to recognition sequences. Several bacterial species have a GlnR/TnrA binding site upstream of the nif cluster. Altering the binding of FBI-GS and GlnR may alter the activity of the nif pathway.

Exemplary engineered genetic variations include a mutation in the gene of interest that may improve the function of the protein encoded by the gene; a constitutionally active promoter that can replace the endogenous promoter of the gene of interest to increase the expression of the gene; a mutation that will inactivate the gene of interest; the insertion of a promoter from within the host's genome into a heterologous location, e.g. insertion of the promoter into a gene that results in inactivation of said gene and upregulation of a downstream gene; and the like. The mutations can be point mutations, insertions, and/or deletions (full or partial deletion of the gene). For example, in some embodiments, to improve the nitrogen fixation activity of the host microbe, a genetic variation may comprise an inactivating mutation of the nifL gene (negative regulator of nitrogen fixation pathway) and/or comprise replacing the endogenous promoter of the nifA and/or nifH gene (nitrogenase iron protein that catalyzes a key reaction to fix atmospheric nitrogen) with a constitutionally active promoter that will drive the expression of the nifA and/or nifH gene constitutively.

In some embodiments, the engineered microbes comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

In some aspects, the engineered microbes are bacteria capable of fixing atmospheric nitrogen in the presence of exogenous nitrogen.

Additional examples of microorganisms and genetic modifications suitable for the microorganisms for use with the compositions and methods of the present disclosure may be found in International Patent Application No. PCT/US22/33002, and International Patent publication Nos. WO/2020/006246A1, WO/2020/118111A1, WO/2021/146209A1, WO/2021/222643A1, and WO2020/014498, the contents of which are herein incorporated by reference in their entirety.

### Compositions

In some embodiments, the compositions disclosed herein are pre-mixed for storage or use as a liquid (e.g., direct application to soil or other surface). The microorganisms mixed with the compositions disclosed herein may be in a liquid or powder form, or may be reconstituted from a powder to a liquid form prior to mixing with the compositions disclosed herein. In some embodiments, the engineered microbes are provided as a microbial composition. In some aspects, the microbial composition has been lyophilized and is provided in a dry powder form. In some aspects, the dry powder has been agglomerated to produce granules. In some aspects, the microbial composition is provided in a liquid form. Additional examples of liquid formulations suitable for the microorganisms for use with the compositions and methods of the present disclosure may be found in International Patent publication No. WO/2021/222643, the content of which is herein incorporated reference in its entirety.

In some aspects, the plurality of engineered bacteria to agricultural biological ratio is between 1:1 and 1:10 by percent volume. In some aspects, the plurality of engineered bacteria to agricultural biological ratio is between 1:1 and 1:5.

In some aspects, the plurality of engineered bacteria are a powder and are reconstituted to a liquid prior to mixture with the agricultural biological at a powder: reconstituted liquid ratio of between 1:4 and 3:10 by weight to volume, including all ranges and subranges therebetween. In some embodiments, the powdered microbes are added directly to the agricultural biological at ratio of 1:1, 1:2, 1:3, 1:4, 1:5, 1:6, 1:7, 1:8, 1:9, 1:10, 1:11, 1:12, 1:15, 1:16, 1:17, 1:18, 1:19, 1:20, 1:21, 1:22, 1:23, 1:24, 1:25, 1:26, 1:27, 1:28, 1:29, or 1:30 by weight to volume (grams to mL).

In some embodiments, the compositions of the present disclosure comprise powdered engineered bacteria at a % weight (grams to mL) to volume of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, including all ranges and subranges therebetween.

In some aspects the plurality of engineered bacteria comprise between 1.0×10⁴ and 1.0×10¹² CFU/mL of the total volume of the mixed composition. In some aspects, the plurality of engineered bacteria are at an initial concentration of 10⁴ to 10¹² CFU/ml. In some aspects the plurality of engineered bacteria are at an initial concentration of 108 to 10¹⁰ CFU/ml. In some aspects the plurality of engineered bacteria are at an initial concentration of about 10⁴, 10⁵, 10⁶, 10⁷, 10⁸, 10⁹, 10¹⁰, 10¹¹, or 10¹² CFU/mL. In some aspects, the plurality of engineered bacteria are at an initial concentration of about 10⁸ CFU/mL. In some aspects, the plurality of engineered bacteria are at an initial concentration of about 10⁹ CFU/mL. In some aspects, the plurality of engineered bacteria are at an initial concentration of about 10¹⁰ CFU/mL. In some aspects, the plurality of engineered bacteria are at an initial concentration of about 10¹¹ CFU/mL. In some aspects, the plurality of engineered bacteria are at an initial concentration of about 10¹² CFU/mL.

In some aspects, the plurality of engineered bacteria are liquid and present in the composition at approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70% (v/v), including all ranges and subranges therebetween.

In some embodiments, agricultural biological comprises at least about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% by weight of the composition.

In some embodiments, the compositions disclosed herein comprises at least one of a polymer, sugar, sugar alcohol, biofilm, and isolated biofilm compositions. In some embodiments, the compositions disclosed herein comprises a stabilizer, buffer, bulking agent, anticaking agent, dispersant, or any combination thereof.

### Buffering Agents

In some embodiments, the compositions disclosed herein comprise a chemical buffer or buffering agent. The buffering agent prevents fluctuations in the pH of the composition, and thus prevents toxic levels of acidity or basicity for microorganisms.

In some embodiments, the chemical buffer maintains the pH of the composition in the pH range of pH 5-9, pH 5-8, pH 5-7, pH 5-6, pH 6-9, pH 6-8, pH 6-7, pH 7-9, or pH 7-8. In some aspects, the chemical buffer maintains the composition at a neutral pH. In some aspects, the chemical buffer comprises potassium phosphate. In some aspects, the chemical buffer is a mixture of monopotassium phosphate (KH₂PO₄) and dipotassium phosphate (K₂HPO₄). While K₂HPO₄ and KH₂PO₄ were used as the buffering salts in the example compositions disclosed herein, one skilled in the art will appreciate that, in some embodiments, other chemical buffers capable of maintaining a neutral or approximately neutral pH may be used.

Non-limiting examples of buffering agents include potassium phosphates, sodium citrate, ascorbate, succinate, lactate, citric acid, boric acid, borax, hydrochloric acid, disodium hydrogen phosphate, acetic acid, formic acid, glycine, bicarbonate, phosphate, tartaric acid, Tris-glycine, Tris-NaCl, Tris-ethylenediamine tetraacetic acid (“EDTA”), Tris-borate, Tris-borate-EDTA, Tris-acteate-EDTA (“TAB”), Tris-buffered saline, 4-(2-hydroxyethyl)-1-piperazineethanesulfonic acid (“HEPES”), 3-(N-morpholino) propanesulfonic acid (“MOPS”), piperazine-1,4-bis(2-ethanesulfonic acid) (“PIPES”), 2-(N-morpholino) ethanesulfonic acid (“MES”), and phosphate buffered saline (“PBS”). Table 5 also provides exemplary buffering agents as well as their pKa values and useful pH ranges.

In some aspects the chemical buffer is at approximately between 0.25% and 1.5% of the total composition volume. In some aspects the chemical buffer is at approximately between 1.5% and 3% of the total composition volume. In some aspects the chemical buffer is at approximately between 3% and 6% of the total composition volume. In some aspects the chemical buffer is at approximately between 6% and 9% of the total composition volume. In some aspects the chemical buffer is at greater than 9% of the total composition volume.

In some aspects, the chemical buffer is present in the composition at approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% (w/w), including all ranges and subranges therebetween.

In some aspects, the chemical buffer is present in the composition at approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% (v/v), including all ranges and subranges therebetween.

TABLE 5
Exemplary buffering agents

| | pKa at | Useful pH |
|---|---|---|
| Common name and/or chemical name | 25° C. | range |
| ACES | 6.78 | 6.1-7.5 |
| Acetic acid (Ethanoic acid) | 4.8 | 3.8-5.8 |
| ADA | 6.59 | 6.0-7.2 |
| AMP | 9.7 | 9.0-10.5 |
| AMPD | 8.8 | 7.8-9.7 |
| AMPSO | 9 | 8.3-9.7 |
| BES | 7.09 | 6.4-7.8 |
| Bicine (2-(bis(2-hydroxyethyl)amino)acetic acid) | 8.35 | 7.6-9.0 |
| Bis-Tris | 6.5 | 5.8-7.2 |
| Bis-Tris Propane | 6.8, 9.0 | 6.3-9.5 |
| Boric acid | 9.24 | 8.25-10.25 |
| CABS | 10.7 | 10.0-11.4 |
| Cacodylate (dimethylarsenic acid) | 6.27 | 5.0-7.4 |
| CAPS | 10.4 | 9.7-11.1 |
| CAPSO | 9.6 | 8.9-10.3 |
| CHES (N-Cyclohexyl-2-aminoethanesulfonic acid) | 9.3 | 8.3-10.3 |
| Citric acid (2-Hydroxypropane-1,2,3-tricarboxylic acid) | 3.13, 4.76, 6.40 | 2.1-7.4 |
| DIPSO | 7.6 | 7.0-8.2 |
| EPPS | 8 | 7.3-8.7 |
| Gly-Gly | 8.2 | 7.5-8.9 |
| HEPBS | 8.3 | 7.6-9.0 |
| HEPES (4-(2-hydroxyethyl)-1-piperazineethanesulfonic | 7.48 | 2.5-3.5 or 6.8-8.2 |
| acid) | | |
| HEPPSO | 7.8 | 7.1-8.5 |
| KH₂PO₄ (Monopotassium phosphate) | 7.2 | 6.2-8.2 |
| K₂HPO₄, | 12.4 | 8.7-9.4 |
| K₃PO₄ | | 11.5-12.3 |
| MES (2-(N-morpholino)ethanesulfonic acid) | 6.15 | 5.5-6.7 |
| MOBS | 7.6 | 6.9-8.3 |
| MOPS (3-(N-morpholino)propanesulfonic acid) | 7.2 | 6.5-7.9 |
| MOPSO | 6.9 | 6.2-7.6 |
| PBS or high buffering capacity PBS | | 5.8-8.0 |
| PIPES | 6.76 | 6.1-7.5 |
| PIPES (piperazine-N,N′-bis(2-ethanesulfonic acid)) | 6.76 | 6.1-7.5 |
| POPSO | 7.8 | 7.2-8.5 |
| TABS | 8.9 | 8.2-9.6 |
| TAPS ([tris(hydroxymethyl)methylamino]propanesulfonic | 8.43 | 7.7-9.1 |
| acid) | | |
| TAPSO (3-[N-tris(hydroxymethyl)methylamino]-2- | 7.635 | 7.0-8.2 |
| hydroxypropanesulfonic acid) | | |
| TEA | 7.8 | 7.3-8.3 |
| TES (2-[[1,3-dihydroxy-2-(hydroxymethyl)propan-2- | 7.4 | 6.8-8.2 |
| yl]amino]ethanesulfonic acid) | | |
| Tricine (N-[tris(hydroxymethyl)methyl]glycine) | 8.05 | 7.4-8.8 |
| Tris (tris(hydroxymethyl)aminomethane) or (2-amino-2- | 8.07 | 7.1-9.1 |
| (hydroxymethyl)propane-1,3-diol) | | |

Additional buffers and instructions on how to prepare them can be found in, e.g., “Common Buffers and Stock Solutions” (2011) Current Protocols in Nucleic Acid Chemistry, A.2A.1-A.2A.14 and in the Sigma Aldrich “Buffer Reference Center” available on the world wide web at sigmaaldrich.com/life-science/core-bioreagents/biological-buffers/learning-center/buffer-reference-center.html, the contents of each of which are incorporated herein in their entirety. Persons having skill in the art will appreciate that the amount of buffer needed to maintain the desired pH will depend on the buffer used, and the total volume of solution.

### Sugars

In some embodiments, the compositions disclosed herein comprise a sugar. In some aspects, the sugar is selected from the group consisting of monosaccharides, disaccharides, trisaccharides, and polysaccharides. In some aspects, the sugar includes one or more of trehalose, sucrose, dextrose, or glycerol. In some aspects, the sugar is a sugar alcohol or non-reducing sugar. In some aspects, the sugar alcohol is selected from the group consisting of sorbitol, mannitol, galactitol, fucitol, iditol, and inositol. In some aspects, the sugar alcohol is sorbitol.

In some aspects, the sugar or sugar alcohol is present in the composition at approximately 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70% 71%, 72%, 73%, 74%, 75%, 76%, 77%, 78%, 79%, 80%, 81%, 82%, or 83% (w/v), including all ranges and subranges therebetween.

### Polymers

In some embodiments, the compositions disclosed herein comprise a polymer. In some aspects, the polymer is selected from the group consisting of polyvinylpyrrolidone (PVP), polyvinylpyrrolidone-vinyl acetate (PVP-VA), carboxymethyl cellulose (CMC), hydroxypropyl methylcellulose, alginate, and combinations thereof. In some aspects, the polymer is polyvinylpyrrolidone-vinyl acetate (PVP-VA). In some aspects, the polymers, engineered microbes, and formulation parameters found in PCT/US2019/064782 (published as WO 2020/118111 A1) are utilized. Whereby PCT/US2019/064782 (published as WO 2020/118111 A1) is herein incorporated by reference in its entirety.

One skilled in the art will appreciate that a large variety of polymers may be used with the compositions, methods, kits, and systems disclosed herein, for example, synthetic polymers, naturally occurring polymers, copolymers, dry-phase polymers, wet-phase polymers, semi-dry polymers, gel polymers, microporous polymers, emulsion polymers, film-forming polymers, allospheres (polymeric nanomaterials), electrospun polymers, cross-linked polymers, water-soluble polymers, and combinations thereof. In some aspects, the polymer is a water-soluble polymer.

In some aspects, the polymer is a naturally occurring polymer. In some aspects, the polymer is produced by a plant or plant part. In some aspects, the polymer is derived from a plant, plant part, or substance therefrom. In some aspects, the polymer is produced by an animal or animal part. In some aspects, the polymer is derived from an animal, animal part, or substance therefrom. In some aspects, the polymer is produced by a microbe such as an algae, protist, bacterium, or fungus. In some aspects, the polymer is derived from a microbe or a substance therefrom. In some aspects, the polymer is an exopolymer. In some aspects, the polymer is an endopolymer.

In some aspects, the polymer contains only repeating units of one type of monomer. In some aspects, the polymer contains repeating units of more than one type of monomer (copolymer). In some aspects, the polymer structure is linear polymer—a linear polymer. In some aspects, the polymer structure is branched polymer—a branched polymer. In some aspects, the polymer structure is network polymer. In some aspects, the polymer is an interpenetrating network polymer.

In some aspects, the polymer is selected from: polyvinylpyrrolidone, polyvinylpyrrolidone-vinyl acetate copolymer 1-(PVP-VA), 2-Pyrrolidinone, ethenylhexadecyl-, homopolymer, carrageenan, sodium alginate, hydroxypropyl methylcellulose (HPMC), polyethylene glycol, gum arabic, maltodextrin, sodium alginate, alginate, xanthan gum, carboxymethyl cellulose (CMC), sodium-carboxymethyl cellulose (Na-CMC), starch BR-07, starch BR-08, starch, and starch-derivatives, pullulan, chitosan, glycosaminoglycans (GAGs), keratin sulfate GAG, hyaluronic acid GAG, heparin sulfate GAG, chondroitin sulfate GAG, polymerized fibrin, polymethylcrylate, polyacrylic acid, polymethacrylic acid, styrene-butadiene, acrylic, styrene-acrylic, vinyl acetate, tocopheryl polyethylene glycol succinate (TPGS)-based polymer, and poly(lactic-co-glycolic acid) (PLGA), etc.

Additional non-limiting examples of polymers that can be used with the compositions, methods, kits, and systems disclosed herein include: polyvinyl acetates, polyvinyl acetate copolymers, ethylene vinyl acetate (EVA) copolymers, polyvinyl alcohols, polyvinyl alcohol copolymers, celluloses (e.g., ethylcelluloses, methylcelluloses, hydroxymethylcelluloses, hydroxypropylcelluloses, and carboxymethylcelluloses), polyvinylpyrolidones, vinyl chloride, vinylidene chloride copolymers, calcium lignosulfonates, acrylic copolymers, polyvinylacrylates, polyethylene oxide, acylamide polymers and copolymers, polyhydroxyethyl acrylate, methylacrylamide monomers, polychloroprene, acrylamide homo- and copolymers, acrylic acid homo- and copolymer, cellulose, methyl cellulose, ethyl cellulose, carboxymethyl cellulose (sodium and other salts), carboxymethyl hydroxyethyl cellulose, hydroxyethyl cellulose, hydrophobically modified hydroxyethyl cellulose, hydroxypropyl cellulose, hydroxypropyl methylcellulose, water-soluble cellulose ethers, carboxy-vinyl copolymers, alginic acid, polyacrylic acid, sodium polyacrylate, partially and fully hydrolyzed polyvinyl alcohols, partially neutralized polyacrylic acid, polyalkylene glycol, polyvinylpyrrolidone and derivatives, starch and its derivatives, vinylpyrrolidone homo- and copolymers, polyacrylamide, attapulgite, montmorillonite, organically modified montmorillonite clays, alumina, precipitated silica, or any mixture thereof.

In some aspects, the polymer is present in the compositions disclosed herein at a % weight to volume of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, including all ranges and subranges therebetween.

In some aspects, the polymer is present in the compositions disclosed herein at a % (wt/wt) of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, or 70%, including all ranges and subranges therebetween.

In some embodiments, the polymer is present in the compositions disclosed herein at a % (wt/wt) of between about 5% and 20%.

### Stabilizers

A microbial stabilizer is an agent that acts to stabilize a microorganism population within a composition. In some embodiments, the compositions disclosed herein comprise a stabilizer. In some embodiments, the microbial stabilizer decreases or slows the decay rate of the microbial population. In some embodiments, the microbial stabilizer accomplishes this change in the decay rate by maintaining the microorganisms in a semi-dormant state. In a semi-dormant state, microorganisms do not respond to environmental conditions as rapidly as they would in an active state.

In some embodiments, the microbial stabilizer improves microbial survival rate, decreases microbial decay, improves microbial metabolic activity, improves microbial catabolic gene expression, improves the microbial colonization rate, or decreases toxin accumulation.

In some embodiments, the microbial stabilizer increases the survival rate of microbial cells after storage, e.g., after 1, 2, 3, 4, 5, or 6 months of storage. In some embodiments, the log loss of CFU/mL of microbes after the storage period is less than 1. In some embodiments, the log loss is less than 1, 0.9, 0.8, 0.7, 0.6, 0.5, 0.4, 0.3, or 0.2.

In some embodiments, the microbial stabilizer improves the metabolic activity and/or catabolic gene expression of the microorganisms in the composition after the storage period.

In some embodiments, the microbial stabilizer improves the colonization rate of the microorganisms in the agricultural plant.

In some embodiments, the microbial stabilizer decreases toxin accumulation. In some embodiments, the toxin is a direct product or byproduct of nitrogen fixation. In some embodiments, the toxin is ammonia or ammonium. In some embodiments, the toxin is produced during cell growth/division.

In some embodiments, the microbial stabilizer is a sugar. In some embodiments, the microbial stabilizer is a non-reducing sugar. Sugars suitable for use include, but are not limited to, sucrose, oligofructose, glucose and fructose. Monosaccharides suitable for use include, but are not limited to, trehalose, sucrose, dextrose, lactose, melibiose, and lactulose. In some embodiments, the microbial stabilizer is trehalose. In some embodiments, the microbial stabilizer is a polysaccharide. Polysaccharides suitable for use include, but are not limited to, maltodextrin, microcrystalline cellulose, and dextran. Additional carbohydrates suitable for use as microbial stabilizers include, but are not limited to, pentoses (e.g., ribose, xylose), hexoses (e.g., mannose, sorbose), oligosaccharides (e.g., raffinose), and oligofructoses. In some embodiments, the microbial stabilizer is a sugar alcohol. Sugar alcohols suitable for use include, but are not limited to, glycerol, mannitol, and sorbitol.

In some embodiments, the microbial stabilizer is an amino acid. In some embodiments, the microbial stabilizer is glycine, proline, glutamate, or cysteine. In some embodiments, the microbial stabilizer is a protein or protein hydrolysate. Proteins or protein hydrolysates suitable for use as microbial stabilizers within the compositions of the present disclosure include, but are not limited to, malt extract, milk powder, casein, whey powder, and yeast extract. In some embodiments, the microbial stabilizer is skimmed milk, starch, humic acid, chitosan, CMC, corn steep liquor, molasses, paraffin, pinolene, NFSM, MgSO4, liquid growth medium, horse serum, or Ficoll.

In some embodiments, the microbial stabilizer is a desiccant. As used herein, a “desiccant” can include any compound or mixture of compounds that can be classified as a desiccant regardless of whether the compound or compounds are used in such concentrations that they in fact have a desiccating effect on the liquid inoculant. Such desiccants are ideally compatible with the microbial population used, and should promote the ability of the microbial population to survive application on the agricultural plant tissues or the environs thereof and to survive desiccation. Examples of suitable desiccants include one or more of trehalose, sucrose, dextrose, glycerol, and methylene glycol. Other suitable desiccants include, but are not limited to, non-reducing sugars and sugar alcohols (e.g., mannitol or sorbitol).

In some embodiments, the microbial stabilizer also acts as a physical stabilizer. In some embodiments, the substance acting as a microbial stabilizer has properties of a thickening agent and therefore also acts as a physical stabilizer. In some embodiments, a composition of the present disclosure comprising both a physical and a microbial stabilizer does so by comprising the same agent that has characteristics of both types of stabilizer.

In some embodiments, the concentration of microbial stabilizer in the compositions disclosed herein is in the range from about 0.1% w/v to about 30% w/v.

In some embodiments, the compositions disclosed herein comprise a physical stabilizer. As used herein, a “physical stabilizer” refers to a substance that improves the homogeneity of the composition, such that the microbial cells are at a similar density throughout the liquid composition. By increasing homogeneity, the physical stabilizer prevents high concentrations of cells and/or toxins from accumulating in any one sub-volume of the dispersion of live microbes.

In some embodiments, the physical stabilizer increases the viscosity of the dispersion of live microbes. In some embodiments, the physical stabilizer is a thickening agent. In some embodiments, the physical stabilizer is an anti-settling agent. In some embodiments, the physical stabilizer is a suspension aid. In some embodiments, the physical stabilizer acts to maintain microbial cells in suspension, improving the cell's resistance to settle statically and flow under shear or rheological shear-thinning. In some embodiments, a physical stabilizer may also have properties of a microbial stabilizer and vice versa.

In some embodiments, the physical stabilizer is a polysaccharide. Polysaccharides suitable for use as physical stabilizers include, but are not limited to, polyethylene glycol (PEG), xanthan gum, pectin, and alginates. In some embodiments, the physical stabilizer is xanthan gum. In some embodiments, the physical stabilizer is a protein or protein hydrolysate. Proteins or protein hydrolysates suitable for use as physical stabilizers include, but are not limited to, gluten, collagen, gelatin, elastin, keratin, and albumin. In some embodiments, the physical stabilizer is a polymer. Polymers suitable for use as physical stabilizers include, but are not limited to, Carbopol® (CBP) polymers, methylene glycol, polyvinyl alcohol (PVA), polyvinylpyrrolidone (PVP), poyacrylate, hydroxyethyl cellulose, or hydroxypropyl methylcellulose. In some embodiments, the physical stabilizer is a gum or its derivative. Gums and their derivatives suitable for use as physical stabilizers include, but are not limited to, guar gum, gum Arabic, gum tragacanth, xanthan gum, derivitized guar, hydroxypropyl guar, and polysaccharide gums. In some embodiments, the physical stabilizer is a CBP polymer.

### Additional Components of the Compositions, Kits, Methods, and Systems Disclosed Herein

In some embodiments, the compositions, methods, kits, and systems disclosed herein may comprise additional components. These additional components may include protectants and beneficial ingredients including but not limited to animal and bird repellants, attractants, baits, herbicides, herbicide safeners, antidessicants, antitranspirants, frost prevention aids, inoculants, dyes, brighteners, markers, synergists, pigments, UV protectants, antioxidants, leaf polish, pigmentation stimulants and inhibitors, surfactants, moisture retention aids, humic acids and humates, lignins and lignates, bitter flavors, irritants, malodorous ingredients, molluscicides (e.g., slugs and snails), nematicides, rodenticides, defoliants, desiccants, sticky traps, IPM (integrated pest management) lures, chemosterilants, plant defense boosters (harpin protein and chitosan), and other beneficial or detrimental agents applied to the surface of the plant seed or tissue. In some embodiments, multiple active agents are readily formulated within a given agricultural composition, for example, multiple active agents may include two or more of any of the following fungicides, fertilizers, pesticides, herbicides, and any type of active ingredient or class of active ingredient.

In some embodiments, the additional component is one or more of a fertilizer, nitrogen stabilizer, or urease inhibitor. Fertilizers include anhydrous ammonia, urea, ammonium nitrate, and urea-ammonium nitrate (UAN) compositions, among many others. In some embodiments, pop-up fertilization and/or starter fertilization is used in combination with the methods and bacteria of the present disclosure.

In some embodiments, nitrogen stabilizers are used in combination with the methods and bacteria of the present disclosure. Nitrogen stabilizers include nitrapyrin, 2-chloro-6-(trichloromethyl)pyridine, N-SERVE 24, INSTINCT, dicyandiamide (DCD). Urease inhibitors include N-(n-butyl)-thiophosphoric triamide (NBPT), AGROTAIN, AGROTAIN PLUS, and AGROTAIN PLUS SC. Further, the disclosure contemplates utilization of AGROTAIN ADVANCED 1.0, AGROTAIN DRI-MAXX, and AGROTAIN ULTRA.

In some embodiments, stabilized forms of fertilizer can be used. For example, a stabilized form of fertilizer is SUPER U, containing 46% nitrogen in a stabilized, urea-based granule, SUPERU contains urease and nitrification inhibitors to guard from denitrification, leaching, and volatilization. Stabilized and targeted foliar fertilizer such as NITAMIN may also be used herein.

Pop-up fertilizers are commonly used in corn fields. Pop-up fertilization comprises applying a few pounds of nutrients with the seed at planting. Pop-up fertilization is used to increase seedling vigor.

Slow- or controlled-release fertilizer that may be used herein entails: A fertilizer containing a plant nutrient in a form which delays its availability for plant uptake and use after application, or which extends its availability to the plant significantly longer than a reference ‘rapidly available nutrient fertilizer’ such as ammonium nitrate or urea, ammonium phosphate or potassium chloride. Such delay of initial availability or extended time of continued availability may occur by a variety of mechanisms. These include controlled water solubility of the material by semi-permeable coatings, occlusion, protein materials, or other chemical forms, by slow hydrolysis of water-soluble low molecular weight compounds, or by other unknown means.

Stabilized nitrogen fertilizer that may be used herein entails: A fertilizer to which a nitrogen stabilizer has been added. A nitrogen stabilizer is a substance added to a fertilizer which extends the time the nitrogen component of the fertilizer remains in the soil in the urea-N or ammoniacal-N form.

Nitrification inhibitor that may be used herein entails: A substance that inhibits the agricultural biological oxidation of ammoniacal-N to nitrate-N. Some examples include: (1) 2-chloro-6-(trichloromethyl-pyridine), common name Nitrapyrin, manufactured by Dow Chemical; (2) 4-amino-1,2,4-6-triazole-HCl, common name ATC, manufactured by Ishihada Industries; (3) 2,4-diamino-6-trichloro-methyltriazine, common name CI-1580, manufactured by American Cyanamid; (4) Dicyandiamide, common name DCD, manufactured by Showa Denko; (5) Thiourea, common name TU, manufactured by Nitto Ryuso; (6) 1-mercapto-1,2,4-triazole, common name MT, manufactured by Nippon; (7) 2-amino-4-chloro-6-methyl-pyramidine, common name AM, manufactured by Mitsui Toatsu; (8) 3,4-dimethylpyrazole phosphate (DMPP), from BASF; (9) 1-amide-2-thiourea (ASU), from Nitto Chemical Ind.; (10) Ammoniumthiosulphate (ATS); (11) 1H-1,2,4-triazole (HPLC); (12) 5-ethylene oxide-3-trichloro-methly 1,2,4-thiodiazole (Terrazole), from Olin Mathieson; (13) 3-methylpyrazole (3-MP); (14) 1-carbamoyle-3-methyl-pyrazole (CMP); (15) Neem; and (16) DMPP.

Urease inhibitor that may be used herein entails: A substance that inhibits hydrolytic action on urea by the enzyme urease. Thousands of chemicals have been evaluated as soil urease inhibitors (Kiss and Simihaian, 2002). However, only a few of the many compounds tested meet the necessary requirements of being nontoxic, effective at low concentration, stable, and compatible with urea (solid and solutions), degradable in the soil and inexpensive. They can be classified according to their structures and their assumed interaction with the enzyme urease (Watson, 2000, 2005). Four main classes of urease inhibitors have been proposed: (a) reagents which interact with the sulphydryl groups (sulphydryl reagents), (b) hydroxamates, (c) agricultural crop protection chemicals, and (d) structural analogues of urea and related compounds. N-(n-Butyl) thiophosphoric triamide (NBPT), phenylphosphorodiamidate (PPD/PPDA), and hydroquinone are probably the most thoroughly studied urease inhibitors (Kiss and Simihaian, 2002). Research and practical testing has also been carried out with N-(2-nitrophenyl) phosphoric acid triamide (2-NPT) and ammonium thiosulphate (ATS). The organo-phosphorus compounds are structural analogues of urea and are some of the most effective inhibitors of urease activity, blocking the active site of the enzyme (Watson, 2005).

In some embodiments, the compositions, methods, kits, and systems disclosed herein may comprise trace metal ions, such as molybdenum ions, iron ions, manganese ions, or combinations of these ions. The concentration of ions in examples of compositions as described herein may between about 0.1 mM and about 50 mM. In some embodiments, the compositions, methods, kits, and systems disclosed herein may comprise additional carriers, besides those which may be included in the microbial compositions. Additional carriers may include beta-glucan, carboxylmethyl cellulose (CMC), bacterial extracellular polymeric substance (EPS), sugar, trehalose, maltose, animal milk, milk powder, or other suitable carriers. In some embodiments, peat or planting materials can be used as a carrier, or biopolymers in which a composition is entrapped in the biopolymer can be used as a carrier.

Additional components for inclusion in the compositions, methods, kits, and systems disclosed herein may be found in International Patent Publication No. WO/2020/006064A3, and International Patent Application No. PCT/US22/33002, the contents of which are herein incorporated by reference in their entirety for all purposes.

### Kits

The ingredients for the compositions disclosed herein may be packaged together as a kit. In some embodiments, the disclosure relates to kits comprising a plurality of engineered bacteria having at least one genetic variation introduced into at least one gene, or non-coding polynucleotide, of the nitrogen fixation or assimilation genetic regulatory network; and an agricultural biological.

In some embodiments a dry microbial powder from one or more species of engineered bacteria could be encased in commercial-grade water-soluble packaging (commonly seen in the detergent industry) and provided in a kit with an agricultural biological. In some embodiments, the plurality of engineered bacteria may be provided in a kit with an aqueous solution of microbes. The end-user would then mix the components of the kit prior to use on a plant, for example, as a seed coating, or an in-furrow treatment.

In some aspects, all of the ingredients for the composition are provided in a dry form, with the end-user combining with water or another liquid to create a liquid composition. In some aspects, parts of the composition are provided premixed in a liquid form.

In some aspects, and the kit further comprises a buffer for reconstitution of the powdered plurality of engineered bacteria. In some aspects, the ingredients for the buffer are provided in a dry form to be mixed by the end user. In some aspects, the ingredients for the buffer are provided pre-mixed in a liquid form.

In some aspects, the kits disclosed herein may comprise powder forms of microbes and or dried ingredients encased in a safe, convenient and eco-friendly water-soluble package. In some embodiments, the disclosure provides the water-soluble packages described in any one of: U.S. Pat. Nos. 7,357,891, 8,617,589, WO 2014/202412, WO 2014/202412, WO 2010/0088112,

EP 1375637, EP 1394065, and US 2001/0033883, the packages comprising any one or more of the dehydrated microbes disclosed herein.

In some embodiments, the water-soluble packages disclosed herein improve the shelf stability of the microbes contained therein. In some embodiments, the microbes in the water-soluble packages disclosed herein have improved shelf stability relative to comparable liquid formulations, dry powders or granules comprising the same microbes which are not encapsulated by the water-soluble packages disclosed herein.

As used herein, “encapsulating” refers to enclosing the microbes, compositions, or specific components of the kits of the present disclosure within water-soluble film package. Encapsulation can be done by any method known in the art for the purpose, or any method that can be conceived to result in the microbes, compositions, or specific components of the kits being encapsulated within the package.

When the water-soluble packages disclosed herein are brought into contact with a liquid (such as, water or an aqueous solution), the package disintegrates, releasing the powder microbes and/or ingredients contained therein into the liquid, thereby forming a liquid that can be applied to seeds or plant propagating material and/or mixed with the compositions disclosed herein to generate a seed treatment coating.

The use of water-soluble packages has several advantages. First, the use of the water-soluble packages disclosed herein obviates the need for direct handling of the dry microbial powder by the end-user, such as a seed treater or a farmer, thus eliminating any real or perceived safety concerns due to the potential inhalation or contact of the microbial powder with skin or eye. Second, the water-soluble packages disclosed herein can be designed to contain a standardized unit of microbes for simplified dosing. Third, the use of the water-soluble packages disclosed herein can promote uniform dispersion of the powdered microbe. For instance, in some embodiments, the components of the water-soluble packages help stabilize the microbes during dry storage and improve dispersion of the microbe in liquids. Therefore, the use of the water-soluble packages promotes consistency in application results.

Fourth, the water-soluble packages disclosed herein enhance the shelf life of the microbes contained therein, since they provide an effective barrier between the microbial powder, and moisture and/or oxygen. Fifth, the water-soluble packages are environment-friendly and reduce packaging waste, while having the potential to be aesthetically pleasing. Sixth, they allow for single dose administration of multiple components that may not be amenable to comingling during storage, or which benefit from different administration timings. Finally, one or more components of the water-soluble packages disclosed herein (such as, polyvinyl alcohols) are released into the dispersion of live microbes upon contact with the liquid. Such components may enhance the survival of the microbes in the dispersion and/or on seed, when the dispersion is applied as a seed treatment.

The water-soluble packages disclosed herein enable the co-administration of components that may not be amenable to being in contact with each other prior to the time of administration, during storage, and/or for long periods of time. Furthermore, when the disclosed agricultural components are brought in contact with a liquid to form a dispersion that can be applied to plants or plant parts, it may be desirable to bring different components in contact with each other in a timed and/or ordered fashion. This kind of regulation of the contact (as well as the timing and/or the order of the contact) between the components can be achieved using the compartmentalization of the disclosed water-soluble packages, as described below.

In some embodiments, contact among the separate ingredients of the composition is prevented until use by placing, for example, the ingredients in separate compartments of the disclosed packages. In some embodiments, the water-soluble package comprises two or more compartment(s).

In some embodiments, the biological of the kit is a biostimulant, biopesticide, or biofertilizer. In some embodiments, the biological is a biostimulant. In some embodiments, the biological of the kit is a biopesticide. In some embodiments, the biological of the kit is a biofertilizer.

In some embodiments, the biological of the kit is a biostimulant that comprises humic substances, hormones, cell signaling molecules, seaweed extract, and/or amino acids. In some embodiments, the biological of the kit is biopesticide selected from the group consisting of a biochemical pesticide, a microbial pesticide, and a plant-incorporated-protectants pesticide. In some embodiments, the biological of the kit is a biofertilizer selected from the group consisting of a bacterial, algal, and fungal biofertilizer. In some embodiments, the biological of the kit is a biofertilizer that comprises at least one of a nitrogen fixer, a phosphate solubilizer, a nutrient mobilizer, plant growth-promoting bacteria, and plant growth-regulating bacteria.

In some embodiments, the agricultural biological of the kit comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

In some embodiments, the agricultural biological of the kit comprises a mycorrhizal fungi selected from Glomus intraradices, Glomus mosseae, Glomus aggregatum, Glomus etunicatum, Glomus clarus, and Rhizophagus intraradices.

In some embodiments, the agricultural biological of the kit comprises a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

In some embodiments, the agricultural biological of the kit comprises a species of Gluconacetobacter selected from G. azotocaptans, G. diazotrophicus, G. johannae, and G. sacchari.

In some embodiments, the agricultural biological of the kit comprises Azotobacter vinelandii and Clostridium pasteurianum.

In some embodiments, the agricultural biological of the kit comprises a mycorrhizal fungi, Beauveria bassiana, Azospirillum sp., Azotobacter sp., and Rhodopseudomonas palustris.

In some embodiments, the agricultural biological of the kit comprises a species of Bacillus selected from B. subtilis, B. simplex, B. methylotrophicus, B. amyloliquefaciens, B. megaterium, and B. licheniformis.

In some embodiments, the agricultural biological of the kit comprises a mycorrhizal fungi, Clostridium sp., and Azotobacter sp.

In some embodiments, the agricultural biological of the kit comprises Bacillus amyloliquefaciens and Trichoderma virens.

In some embodiments, the agricultural biological of the kit comprises a species of Trichoderma selected from T. harzianum, T. atroviride, T. asperellum, and T. hamatum.

In some embodiments, the agricultural biological of the kit comprises Ochrobactrum anthropic, Bacillus Subtillus, and Bacillus simplex.

In some embodiments, the agricultural biological of the kit comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

In some embodiments, the agricultural biological of the kit comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii.

In some embodiments, the agricultural biological of the kit comprises a seaweed extract from Reynoutria sachalinensis and/or Ascophyllum nodosum.

In some embodiments, the agricultural biological of the kit is a biostimulant comprising a compound of Formula (2) or a salt, solvent, or isomer thereof.

In some embodiments, the agricultural biological of the kit is a biostimulant comprising a compound of Formula (3) or a salt, solvent, or isomer thereof.

In some embodiments, the agricultural biological of the kit is a biostimulant comprising a maltol compound. In some embodiments, the agricultural biological of the kit is a biostimulant comprising a lactone compound. In some embodiments, the agricultural biological of the kit is a biostimulant comprising a maltol lactone compound.

In some embodiments, the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

In some embodiments, the plurality of engineered bacteria of the kit comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

In some embodiments, the engineered bacteria of the kit comprise at least one microbial species capable of fixing atmospheric nitrogen in the presence of exogenous nitrogen. In some embodiments, the engineered bacteria of the kit comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

In some embodiments, the engineered bacteria of the kit comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

In some embodiments, the engineered bacteria of the kit comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

In some embodiments, the engineered bacteria of the kit do not comprise genetic material that originates from a different species than the remodeled bacteria. In some embodiments, the engineered bacteria of the kit are epiphytes. In some embodiments, the engineered bacteria of the kit are endophytes. In some embodiments, the engineered bacteria of the kit are rhizophytes.

In some embodiments, the engineered bacteria of the kit comprise at least one microbial species that is a non-intergeneric remodeled microbial species comprising an introduced control sequence operably linked to at least one gene of the nitrogen fixation or assimilation genetic regulatory network.

In some embodiments, the engineered bacteria of the kit comprise at least one microbial species that is a non-intergeneric remodeled microbial species comprising a heterologous promoter operably linked to at least one gene of the nitrogen fixation or assimilation genetic regulatory network.

In some embodiments, the engineered bacteria of the kit comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

In some embodiments, the engineered bacteria of the kit comprise at least one microbial species that is a non-intergeneric remodeled microbial species having a mutated nifL gene that has been altered to comprise a heterologous promoter inserted into said nifL gene.

In some embodiments, the engineered bacteria of the kit comprise at least one microbial species that is a non-intergeneric remodeled microbial species comprising a mutated glnE gene that results in a truncated GlnE protein lacking an adenylyl-removing (AR) domain.

In some embodiments, the engineered bacteria of the kit comprise at least one microbial species that is a non-intergeneric remodeled microbial species comprising a mutated amtB gene that results in the lack of expression of said amtB gene.

In some embodiments, the engineered bacteria of the kit comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

In some embodiments, the engineered bacteria of the kit comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

In some embodiments, the engineered bacteria of the kit comprise Kosakonia sacchari. In some embodiments, the engineered bacteria of the kit comprise Kosakonia sacchari PTA-126743.

In some embodiments, the engineered bacteria of the kit comprise Klebsiella variicola. In some embodiments, the engineered bacteria of the kit comprise Klebsiella variicola PTA-126740.

In some embodiments, the engineered bacteria of the kit comprise Klebsiella variicola PTA-126740 and Kosakonia sacchari PTA-126743.

In some embodiments, the engineered bacteria of the kit are a liquid formulation. In some embodiments, the engineered bacteria of the kit are reconstituted from a previous powder formulation.

In some embodiments, the engineered bacteria of the kit are at a concentration of between about 1.0×10⁴ and about 1.0×10¹² CFU/mL of the total volume of the composition. In some embodiments, the engineered bacteria of the kit are a powder formulation of

lyophilized microbes.

In some embodiments, the engineered bacteria and/or the agricultural biological are encapsulated within a water-soluble package.

In some embodiments, the engineered bacteria and/or the agricultural biological comprise at least one of a polymer, buffer, sugar, sugar alcohol, stabilizer, bulking agent, anti-caking agent, dispersant, biofilm, and isolated biofilm composition.

In some embodiments, the engineered bacteria and/or the agricultural biological comprise a sugar or sugar alcohol. In some embodiments, the sugar or sugar alcohol is selected from sucrose, lactose, trehalose, sorbitol, mannitol, galactitol, fucitol, iditol, inositol, and combinations thereof.

In some embodiments, the engineered bacteria and/or the agricultural biological comprise a buffer. In some embodiments, the buffer is selected from potassium phosphate, dipotassium phosphate, monopotassium phosphate, and combinations thereof.

In some embodiments, the engineered bacteria and/or the agricultural biological comprise a polymer. In some embodiments, the polymer is selected from polyvinylpyrrolidone (PVP), polyvinylpyrrolidone-vinyl acetate (PVP-VA), carboxymethyl cellulose (CMC), hydroxypropyl methylcellulose, alginate, and combinations thereof.

In some embodiments, the engineered bacteria and/or the agricultural biological comprise an agriculturally acceptable adjuvant, excipient, or carrier.

In some embodiments, the kit further comprises instructions for mixing, and/or applying the engineered bacteria and the agricultural biological to a plant or to an area in which a plant will be grown or is growing.

### Farm Administrative Systems

In some embodiments, the disclosure provides a farm administration system, comprising: a) a plurality of engineered bacteria having at least one genetic variation introduced into at least one gene, or non-coding polynucleotide, of the nitrogen fixation or assimilation genetic regulatory network; b) an agricultural biological; and c) instructions for using a) and b) to treat a plant.

In some embodiments, the agricultural biological of the farm administration system is a biostimulant, biopesticide, or biofertilizer.

In some embodiments, the agricultural biological of the farm administration system is a biostimulant that comprises humic substances, hormones, cell signaling molecules, seaweed extract, and/or amino acids.

In some embodiments, the agricultural biological of the farm administration system is a biopesticide selected from the group consisting of a biochemical pesticide, a microbial pesticide, and a plant-incorporated-protectants pesticide.

In some embodiments, the agricultural biological of the farm administration system is a biofertilizer selected from the group consisting of a bacterial, algal, and fungal biofertilizer.

In some embodiments, the agricultural biological of the farm administration system is a biofertilizer that comprises at least one of a nitrogen fixer, a phosphate solubilizer, a nutrient mobilizer, plant growth-promoting bacteria, and plant growth-regulating bacteria.

In some embodiments, the agricultural biological of the farm administration system comprise sone or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

In some embodiments, the agricultural biological of the farm administration system is a mycorrhizal fungi selected from Glomus intraradices, Glomus mosseae, Glomus aggregatum, Glomus etunicatum, Glomus clarus, and Rhizophagus intraradices.

In some embodiments, the agricultural biological of the farm administration system is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

In some embodiments, the agricultural biological of the farm administration system is a species of Gluconacetobacter selected from G. azotocaptans, G. diazotrophicus, G. johannae, and G. sacchari.

In some embodiments, the agricultural biological of the farm administration system comprises Azotobacter vinelandii and Clostridium pasteurianum.

In some embodiments, the agricultural biological of the farm administration system comprises a mycorrhizal fungi, Beauveria bassiana, Azospirillum sp., Azotobacter sp., and Rhodopseudomonas palustris.

In some embodiments, the agricultural biological of the farm administration system is a species of Bacillus selected from B. subtilis, B. simplex, B. methylotrophicus, B. amyloliquefaciens, B. megaterium, and B. licheniformis.

In some embodiments, the agricultural biological of the farm administration system comprises a mycorrhizal fungi, Clostridium sp., and Azotobacter sp. In some embodiments, the agricultural biological of the farm administration system comprises Bacillus amyloliquefaciens and Trichoderma virens. In some embodiments, the agricultural biological of the farm administration system is a species of Trichoderma selected from T. harzianum, T. atroviride, T. asperellum, and T. hamatum. In some embodiments, the agricultural biological of the farm administration system comprises Ochrobactrum anthropic, Bacillus Subtillus, and Bacillus simplex.

In some embodiments, the agricultural biological of the farm administration system comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

In some embodiments, the agricultural biological of the farm administration system comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii.

In some embodiments, the agricultural biological of the farm administration system is a seaweed extract from Reynoutria sachalinensis and/or Ascophyllum nodosum.

In some embodiments, the agricultural biological of the farm administration system is a biostimulant comprising a compound of Formula (2) or a salt, solvent, or isomer thereof, as disclosed herein.

In some embodiments, the agricultural biological of the farm administration system is a biostimulant comprising a compound of Formula (3), as disclosed herein.

In some embodiments, the agricultural biological of the farm administration system is a biostimulant comprising a maltol compound. In some embodiments, the agricultural biological of the farm administration system is a biostimulant comprising a lactone compound. In some embodiments, the agricultural biological of the farm administration system is a biostimulant comprising a maltol lactone compound.

In some embodiments, the plurality of engineered bacteria of the farm administration system are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

In some embodiments, the plurality of engineered bacteria of the farm administration system comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

In some embodiments, the plurality of engineered bacteria of the farm administration system comprise at least one microbial species capable of fixing atmospheric nitrogen in the presence of exogenous nitrogen.

In some embodiments, the plurality of engineered bacteria of the farm administration system comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

In some embodiments, the plurality of engineered bacteria of the farm administration system comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

In some embodiments, the plurality of engineered bacteria of the farm administration system comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

In some embodiments, the plurality of engineered bacteria of the farm administration system do not comprise genetic material that originates from a different species than the remodeled bacteria.

In some embodiments, the plurality of engineered bacteria of the farm administration system comprise an epiphyte. In some embodiments, the plurality of engineered bacteria of the farm administration system comprise an endophyte. In some embodiments, the plurality of engineered bacteria of the farm administration system comprise a rhizophyte.

In some embodiments, the plurality of engineered bacteria of the farm administration system comprise at least one microbial species that is a non-intergeneric remodeled microbial species comprising an introduced control sequence operably linked to at least one gene of the nitrogen fixation or assimilation genetic regulatory network.

In some embodiments, the plurality of engineered bacteria of the farm administration system comprise at least one microbial species that is a non-intergeneric remodeled microbial species comprising a heterologous promoter operably linked to at least one gene of the nitrogen fixation or assimilation genetic regulatory network.

In some embodiments, the plurality of engineered bacteria of the farm administration system comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

In some embodiments, the plurality of engineered bacteria of the farm administration system comprise at least one microbial species that is a non-intergeneric remodeled microbial species having a mutated nifL gene that has been altered to comprise a heterologous promoter inserted into said nifL gene.

In some embodiments, the plurality of engineered bacteria of the farm administration system comprise at least one microbial species that is a non-intergeneric remodeled microbial species comprising a mutated glnE gene that results in a truncated GlnE protein lacking an adenylyl-removing (AR) domain.

In some embodiments, the plurality of engineered bacteria of the farm administration system comprise at least one microbial species that is a non-intergeneric remodeled microbial species comprising a mutated amtB gene that results in the lack of expression of said amtB gene.

In some embodiments, the plurality of engineered bacteria of the farm administration system comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

In some embodiments, the plurality of engineered bacteria of the farm administration system comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

In some embodiments, the plurality of engineered bacteria of the farm administration system comprise Kosakonia sacchari. In some embodiments, the plurality of engineered bacteria of the farm administration system comprise Kosakonia sacchari PTA-126743. In some embodiments, the plurality of engineered bacteria of the farm administration system comprise Klebsiella variicola. In some embodiments, the plurality of engineered bacteria of the farm administration system comprise Klebsiella variicola PTA-126740. In some embodiments, the plurality of engineered bacteria of the farm administration system comprise Klebsiella variicola PTA-126740 and Kosakonia sacchari PTA-126743.

In some embodiments, the plurality of engineered bacteria of the farm administration system are a liquid formulation. In some embodiments, the plurality of engineered bacteria of the farm administration system are reconstituted from a previous powder formulation.

In some embodiments, the plurality of engineered bacteria of the farm administration system are at a concentration of between about 1.0×10⁴ and about 1.0×10¹² CFU/mL of the total volume of the composition.

In some embodiments, the plurality of engineered bacteria of the farm administration system are a powder formulation of lyophilized microbes. In some embodiments, the plurality of engineered bacteria of the farm administration system and/or the agricultural biological are encapsulated within a water-soluble package.

In some embodiments, the instructions of the farm administration system comprise a recommended dose of the engineered bacteria and the agricultural biological to treat a plant species of Hordeum, Oryza, Zea, Sorghum, Brassica, or Triticeae. In some embodiments, the plant is corn. In some embodiments, the plant is soybean. In some embodiments, the plant is rice. In some embodiments, the plant is wheat. In some embodiments, the plant is rapeseed. In some embodiments, the plant is sweet corn, flint corn, popcorn, dent corn, pod corn, or flour corn.

In some embodiments, the instructions of the farm administration system comprise recommended application methods. In some embodiments, the recommended application methods of the instructions are selected from in-furrow, seed treatment, seedling root dip, broadcast, and foliar spray.

In some embodiments, the instructions of the farm administration system comprise a recommended order of applying the engineered bacteria and the agricultural biological.

### Methods of Treating a Plant

In some embodiments, the disclosure teaches a method of treating a plant, comprising applying a plurality of engineered bacteria having at least one genetic variation introduced into at least one gene, or non-coding polynucleotide, of the nitrogen fixation or assimilation genetic regulatory network to a plant or to an area in which a plant will be grown or is growing; and applying an agricultural biological to the plant or to an area in which a plant will be grown or is growing.

In some embodiments, the method comprises applying the compositions disclosed herein. In some embodiments, the method comprises using the kits disclosed herein. In some embodiments, the method comprises using the farm administrative systems disclosed herein.

In some embodiments, the agricultural biological used in the methods disclosed herein is a biostimulant, biopesticide, or biofertilizer.

In some embodiments, the biological used in the methods disclosed herein is a biostimulant that comprises humic substances, hormones, cell signaling molecules, seaweed extract, and/or amino acids.

In some embodiments, the biological used in the methods disclosed herein is biopesticide selected from the group consisting of a biochemical pesticide, a microbial pesticide, and a plant-incorporated-protectants pesticide.

In some embodiments, the biological used in the methods disclosed herein is a biofertilizer selected from the group consisting of a bacterial, algal, and fungal biofertilizer. In some embodiments, the biological is a biofertilizer that comprises at least one of a nitrogen fixer, a phosphate solubilizer, a nutrient mobilizer, plant growth-promoting bacteria, and plant growth-regulating bacteria.

In some embodiments, the agricultural biological used in the methods disclosed herein comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

In some embodiments, the agricultural biological used in the methods disclosed herein is a mycorrhizal fungi selected from Glomus intraradices, Glomus mosseae, Glomus aggregatum, Glomus etunicatum, Glomus clarus, and Rhizophagus intraradices.

In some embodiments, the agricultural biological used in the methods disclosed herein is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

In some embodiments, the agricultural biological used in the methods disclosed herein is a species of Gluconacetobacter selected from G. azotocaptans, G. diazotrophicus, G. johannae, and G. sacchari.

In some embodiments, the agricultural biological used in the methods disclosed herein comprises Azotobacter vinelandii and Clostridium pasteurianum.

In some embodiments, the agricultural biological used in the methods disclosed herein comprises a mycorrhizal fungi, Beauveria bassiana, Azospirillum sp., Azotobacter sp., and Rhodopseudomonas palustris.

In some embodiments, the agricultural biological used in the methods disclosed herein is a species of Bacillus selected from B. subtilis, B. simplex, B. methylotrophicus, B. amyloliquefaciens, B. megaterium, and B. licheniformis.

In some embodiments, the agricultural biological used in the methods disclosed herein comprises a mycorrhizal fungi, Clostridium sp., and Azotobacter sp. In some embodiments, the agricultural biological used in the methods disclosed herein comprises Bacillus amyloliquefaciens and Trichoderma virens. In some embodiments, the agricultural biological is a species of Trichoderma selected from T. harzianum, T. atroviride, T. asperellum, and T. hamatum. In some embodiments, the agricultural biological comprises Ochrobactrum anthropic, Bacillus Subtillus, and Bacillus simplex.

In some embodiments, the agricultural biological used in the methods disclosed herein comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

In some embodiments, the agricultural biological used in the methods disclosed herein comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii

In some embodiments, the agricultural biological used in the methods disclosed herein is a seaweed extract from Reynoutria sachalinensis and/or Ascophyllum nodosum.

In some embodiments, the biological used in the methods disclosed herein is a biostimulant comprising a compound of Formula (2) or a salt, solvent, or isomer thereof, as disclosed herein. In some embodiments, the biological used in the methods disclosed herein is a biostimulant comprising a compound of Formula (3), as disclosed herein.

In some embodiments, the biological used in the methods disclosed herein is a biostimulant comprising a maltol compound. In some embodiments, the biological is a biostimulant comprising a lactone compound. In some embodiments, the biological is a biostimulant comprising a maltol lactone compound.

In some embodiments, the plurality of non-intergeneric remodeled bacteria used in the methods disclosed herein are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

In some embodiments, the plurality of engineered bacteria used in the methods disclosed herein comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

In some embodiments, the engineered bacteria used in the methods disclosed herein comprise at least one microbial species capable of fixing atmospheric nitrogen in the presence of exogenous nitrogen.

In some embodiments, the engineered bacteria used in the methods disclosed herein comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

In some embodiments, the engineered bacteria used in the methods disclosed herein comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

In some embodiments, the engineered bacteria used in the methods disclosed herein comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

In some embodiments, the engineered bacteria used in the methods disclosed herein do not comprise genetic material that originates from a different species than the remodeled bacteria.

In some embodiments, the engineered bacteria used in the methods disclosed herein comprise an epiphyte. In some embodiments, the engineered bacteria used in the methods disclosed herein comprise an endophyte. In some embodiments, the engineered bacteria used in the methods disclosed herein comprise a rhizophyte.

In some embodiments, the engineered bacteria used in the methods disclosed herein comprise at least one microbial species that is a non-intergeneric remodeled microbial species comprising an introduced control sequence operably linked to at least one gene of the nitrogen fixation or assimilation genetic regulatory network.

In some embodiments, the engineered bacteria used in the methods disclosed herein comprise at least one microbial species that is a non-intergeneric remodeled microbial species comprising a heterologous promoter operably linked to at least one gene of the nitrogen fixation or assimilation genetic regulatory network.

In some embodiments, the engineered bacteria used in the methods disclosed herein comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

In some embodiments, the engineered bacteria used in the methods disclosed herein comprise at least one microbial species that is a non-intergeneric remodeled microbial species having a mutated nifL gene that has been altered to comprise a heterologous promoter inserted into said nifL gene.

In some embodiments, the engineered bacteria used in the methods disclosed herein comprise at least one microbial species that is a non-intergeneric remodeled microbial species comprising a mutated glnE gene that results in a truncated GlnE protein lacking an adenylyl-removing (AR) domain.

In some embodiments, the engineered bacteria used in the methods disclosed herein comprise at least one microbial species that is a non-intergeneric remodeled microbial species comprising a mutated amtB gene that results in the lack of expression of said amtB gene.

In some embodiments, the engineered bacteria used in the methods disclosed herein comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

In some embodiments, the engineered bacteria used in the methods disclosed herein comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaß, pehA, otsB, treZ, glsA2, or combinations thereof.

In some embodiments, the engineered bacteria used in the methods disclosed herein comprise Kosakonia sacchari. In some embodiments, the engineered bacteria used in the methods disclosed herein comprise Kosakonia sacchari PTA-126743. In some embodiments, the engineered bacteria used in the methods disclosed herein comprise Klebsiella variicola. In some embodiments, the engineered bacteria used in the methods disclosed herein comprise Klebsiella variicola PTA-126740. In some embodiments, the engineered bacteria used in the methods disclosed herein comprise Klebsiella variicola PTA-126740 and Kosakonia sacchari PTA-126743.

In some embodiments, the engineered bacteria used in the methods disclosed herein are a liquid formulation. In some embodiments, the plurality of engineered bacteria used in the methods disclosed herein are reconstituted from a previous powder formulation.

In some embodiments, the engineered bacteria used in the methods disclosed herein are at a concentration of between about 1.0×10⁴ and about 1.0×10¹² CFU/mL of the total volume of the composition when it is applied to a plant or area in which a plant will be grown or is growing.

In some embodiments, the engineered bacteria used in the methods disclosed herein are a powder formulation of lyophilized microbes. In some embodiments, the engineered bacteria are encapsulated within a water-soluble package.

In some embodiments, the method comprises mixing the engineered bacteria and the agricultural biological prior to application to a plant or to an area in which a plant will be grown or is growing.

In some embodiments, the agricultural biological is applied in-furrow, as a seed treatment, as a seedling root dip, as a broadcast, or as a foliar spray.

In some embodiments, the engineered bacteria are applied in-furrow, as a seed treatment, as a seedling root dip, as a broadcast, or as a foliar spray.

Conventional or otherwise suitable coating equipment or techniques may be used to coat the seeds or plant propagating material with the seed coating treatments described above. Suitable equipment is deemed to include drum coaters, fluidized beds, rotary coaters, side vended pan, tumble mixers and spouted beds, but any suitable equipment or technique may be used. Additionally, various coating machines are available to a person skilled in the art.

In some embodiments, the seed coating comprises engineered bacteria at a concentration of about 1×10⁴ to about 1×10¹¹ CFU per seed at the time of planting, when planted within 28 days of application. In some aspects, the engineered bacteria are at a concentration of about 1×10⁵ to about 1×10⁷ CFU per seed at the time of planting. In some aspects, the engineered bacteria are at a concentration of about 1×10⁶ CFU per seed.

In some aspects, the method comprises coating a seed or plant propagating material with a plurality of engineered bacteria and/or an agricultural biological wherein the seed or other plant propagation material has at least one pre-treatment. In some aspects, the pre-treatment is a plant enhancing agent. In some embodiments, the pre-treatment is an insecticide, herbicide, fungicide, biocide, or nematicide.

In some embodiments, the engineered bacteria and the agricultural biological are applied simultaneously. For example, one may be applied as a root drench and the other as a foliar spray.

In some embodiments, the engineered bacteria and the agricultural biological are applied sequentially. For example, one may be applied as a seed treatment, while the other is applied at a later timepoint as a seedling root dip.

In some embodiments, application of the engineered bacteria and the agricultural biological are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

In some embodiments, application of the engineered bacteria and the agricultural biological are separated by 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days.

In some embodiments, application of the engineered bacteria and the agricultural biological are separated by 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 days.

### Improvement of Plant Traits

The disclosure provides compositions, methods, kits, and systems to improve plant traits and decrease fertilizer use. The synergistic effect of nitrogen fixing microbes and agricultural biologicals can improve a variety of desirable traits in a plant.

In some embodiments, the compositions, methods, kits, and systems increase nitrogen fixation in a plant or plant part compared to control plants, which have not been treated with the compositions, methods, kits, and systems disclosed herein, or which have only been treated with nitrogen fixing microbes and no agricultural biological, and vice versa.

Examples of traits that may be introduced or improved include: root biomass, root length, height, shoot length, leaf number, water use efficiency, overall biomass, yield, fruit size, grain size, photosynthesis rate, tolerance to drought, heat tolerance, salt tolerance, resistance to nematode stress, resistance to a fungal pathogen, resistance to a bacterial pathogen, resistance to a viral pathogen, level of a metabolite, and proteome expression. The desirable traits, including height, overall biomass, root and/or shoot biomass, seed germination, seedling survival, photosynthetic efficiency, transpiration rate, seed/fruit number or mass, plant grain or fruit yield, leaf chlorophyll content, photosynthetic rate, root length, or any combination thereof, can be used to measure growth, and compared with the growth rate of reference agricultural plants (e.g., plants without the improved traits) grown under identical conditions. In some aspects, the compositions, methods, kits, and systems described herein can improve plant traits, such as promoting plant growth, maintaining high chlorophyll content in leaves, increasing fruit or seed numbers, and increasing fruit or seed unit weight. In some aspects, the plant grown from the treated seed or plant material has improved health, yield, stress resistance, growth, or agronomic characteristics relative to a control plant.

Traits that may be improved by the compositions and methods disclosed herein include any observable characteristic of the seed or the plant resulting therefrom, including, for example, growth rate, height, weight, color, taste, smell, changes in the production of one or more compounds by the plant (including for example, metabolites, proteins, drugs, carbohydrates, oils, and any other compounds). In some aspects, the compositions and methods disclosed herein may result in a change in genotypic information (for example, a change in the pattern of plant gene expression such as those associated with increased nitrogen fixation, in response to the microbes). In some aspects, the plants show the absence, suppression or inhibition of a certain feature or trait (such as an undesirable feature or trait) as opposed to the presence of a certain feature or trait (such as a desirable feature or trait).

In some embodiments a wilting of the treated plant is reduced or delayed as compared to an untreated plant, a turgidity of the treated plant is prolonged or maintained as compared to an untreated plant, a loss of one or more petals of the treated plant is reduced or delayed as compared to an untreated plant, a chlorophyll content of the treated plant is maintained as compared to an untreated plant, a loss of the chlorophyll content of the treated plant is reduced or delayed as compared to an untreated plant, a chlorophyll content of the treated plant is increased as compared to an untreated plant, a salinity tolerance of the treated plant is increased as compared to an untreated plant, a water consumption of the treated plant is reduced as compared to an untreated plant, a drought tolerance of the treated plant is increased as compared to an untreated plant, a pest resistance of the treated plant is increased as compared to an untreated plant, a pesticides consumption of the treated plant is reduced as compared to an untreated plant, or any combination thereof.

The trait improved may be nitrogen fixation, including in a plant not previously capable of nitrogen fixation. In some embodiments, enhanced levels of nitrogen fixation are achieved in the presence of fertilizer supplemented with glutamine, ammonia, or other chemical source of nitrogen. Methods for assessing degree of nitrogen fixation are known and may be employed to assess the methods described herein.

In some embodiments, application of the compositions, methods, kits, and systems disclosed herein increases plant nutrient uptake compared to an untreated plant.

In some embodiments, application of the compositions, methods, kits, and systems disclosed herein increases plant available nitrogen, phosphorus, or potassium compared to an untreated plant.

In some embodiments, application of the compositions, methods, kits, and systems disclosed herein increases whole plant nitrogen.

In some embodiments, application of the compositions, methods, kits, and systems disclosed herein result in increased nitrogen fixation activity in a field as compared to un-remodeled bacteria of the same species, in the same field. In some aspects, the field has a fixed nitrogen concentration of at least 0.01 mM. In some aspects, the field has a fixed nitrogen concentration of at least 0.1 mM. In some aspects, the field has a fixed nitrogen concentration of at least 0.5 mM.

In some embodiments, application of the compositions, methods, kits, and systems disclosed herein decreases the amount of fertilizer required compared to an untreated plant.

In some embodiments, application of the compositions, methods, kits, and systems disclosed herein increases plant yield compared to an untreated plant.

In some embodiments, application of the compositions, methods, kits, and systems disclosed herein increases chlorophyll content compared to an untreated plant.

In some embodiments, application of the compositions, methods, kits, and systems disclosed herein increases resistance to an abiotic or biotic stressor. In some aspects, the abiotic stressor is low or high temperature, deficient or excessive water, high salinity, heavy metals, or ultraviolet radiation.

Plant Species for Use with the Disclosed Compositions, Kits, Systems, and Methods

The compositions, methods, kits, and systems disclosed herein can be applied to a number of seeds and plant parts. In some embodiments, the seed or plant part is an agricultural crop. In some embodiments the seed or plant part is a monocot. In some embodiments, the seed or plant part is a dicot. In some embodiments, the seed is a corn seed. In some embodiments, the corn seed comprises a pre-treatment.

It will be understood by one skilled in the art that seed or plant propagating material of any agriculturally important crop could be used with the compositions, methods, kits, and systems disclosed herein, including but not limited to, rice, sorghum, canola, tomato, strawberry, and barley.

In some embodiments, the plant belongs to the genera Hordeum, Oryza, Zea, Brassica, and Triticeae. Non-limiting examples of crop plants include maize, rice, wheat, rapeseed, barley, sorghum, millet, oats, rye triticale, buckwheat, sweet corn, sugar cane, onions, tomatoes, strawberries, asparagus, canola, soybean, potato, vegetables, cereals, and oilseeds. In some embodiments, the plant is a genetically modified organism (GMO), non-GMO, organic, or conventional plant. In some embodiments, the compositions, methods, kits, and systems described herein are suitable for plant tissues from any of a variety of transgenic plants, non-transgenic plants, and hybrid plants thereof.

In some embodiments, the plants are important or interesting for agriculture, horticulture, biomass for the production of biofuel molecules and other chemicals, and/or forestry. Some examples of these plants may include pineapple, banana, coconut, lily, grasspeas and grass; and dicotyledonous plants, such as, for example, peas, alfalfa, tomatillo, melon, chickpea, chicory, clover, kale, lentil, soybean, tobacco, potato, sweet potato, radish, cabbage, rape, apple trees, grape, cotton, sunflower, thale cress, canola, citrus (including orange, mandarin, kumquat, lemon, lime, grapefruit, tangerine, tangelo, citron, and pomelo), pepper, bean, lettuce, Panicum virgatum (switch), Sorghum bicolor (sorghum, sudan), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp. Sorghum spp., Miscanthus spp., Saccharum spp., Erianthus spp., Populus spp., Secale cereale (rye), Salix spp. (willow), Eucalyptus spp. (eucalyptus), Triticosecale spp. (triticum-25 wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brussel sprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea 5 spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), Hordeum vulgare (barley), and Lolium spp. (rye).

In some embodiments, plant tissues or plant parts, e.g., seeds, from a monocotyledonous plant are treated. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales. In some embodiments, the monocotyledonous plant can be selected from the group consisting of a maize, rice, wheat, barley, rapeseed, and sugarcane.

In some embodiments, plant tissues or plant parts, e.g., seeds, from a dicotyledonous plant are treated, including those belonging to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Middles, Juglandales, Lamiales, Laurales, Lecythidales, Leitneriales, Magniolales, Malvales, Myricales, Myrtales, Nymphaeales, Papeverales, Piperales, Plantaginales, Plumb aginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violates. In some embodiments, the dicotyledonous plant can be selected from the group consisting of cotton, soybean, pepper, and tomato.

Additional plants and seeds acceptable for use within the methods and compositions of the present disclosure may be found in International Publication Nos. WO/2020/006246A1 and WO/2020/006064A3, the contents of each of which are herein incorporated by reference in their entirety.

The compositions, methods, kits, and systems described herein are suitable for any of a variety of non-genetically modified maize plants or parts thereof. In some embodiments, the corn is organic. The compositions, methods, kits, and systems described herein are suitable for any non-genetically modified hybrids, varieties, lineages, etc. Corn varieties generally fall under six categories: sweet corn, flint corn, popcorn, dent corn, pod corn, and flour corn.

### Sweet Corn

Yellow su varieties include Earlivee, Early Sunglow, Sundance, Early Golden Bantam, Iochief, Merit, Jubilee, and Golden Cross Bantam. White su varieties include True Platinum, Country Gentleman, Silver Queen, and Stowell's Evergreen. Bicolor su varieties include Sugar & Gold, Quickie, Double Standard, Butter & Sugar, Sugar Dots, Honey & Cream. Multicolor su varieties include Hookers, Triple Play, Painted Hill, Black Mexican/Aztec.

Yellow se varieties include Buttergold, Precocious, Spring Treat, Sugar Buns, Colorow, Kandy King, Bodacious R/M, Tuxedo, Incredible, Merlin, Miracle, and Kandy Korn EH. White se varieties include Spring Snow, Sugar Pearl, Whiteout, Cloud Nine, Alpine, Silver King, and Argent. Bicolor se varieties include Sugar Baby, Fleet, Bon Jour, Trinity, Bi-Licious, Temptation, Luscious, Ambrosia, Accord, Brocade, Lancelot, Precious Gem, Peaches and Cream Mid EH, and Delectable R/M. Multicolor se varieties include Ruby Queen.

Yellow sh2 varieties include Extra Early Super Sweet, Takeoff, Early Xtra Sweet, Raveline, Summer Sweet Yellow, Krispy King, Garrison, Illini Gold, Challenger, Passion, Excel, Jubilee SuperSweet, Illini Xtra Sweet, and Crisp 'N Sweet. White sh2 varieties include Summer Sweet White, Tahoe, Aspen, Treasure, How Sweet It Is, and Camelot. Bicolor sh2 varieties include Summer Sweet Bicolor, Radiance, Honey 'N Pearl, Aloha, Dazzle, Hudson, and Phenomenal.

Yellow sy varieties include Applause, Inferno, Honeytreat, and Honey Select. White sy varieties include Silver Duchess, Cinderella, Mattapoisett, Avalon, and Captivate. Bicolor sy varieties include Pay Dirt, Revelation, Renaissance, Charisma, Synergy, Montauk, Kristine, Serendipity/Providence, and Cameo.

Yellow augmented supersweet varieties include Xtra-Tender 1ddA, Xtra-Tender 11dd, Mirai 131Y, Mirai 130Y, Vision, and Mirai 002. White augmented supersweet varieties include Xtra-Tender 3dda, Xtra-Tender 31dd, Mirai 421W, XTH 3673, and Devotion. Bicolor augmented supersweet varieties include Xtra-Tender 2dda, Xtra-Tender 21dd, Kickoff XR, Mirai 308BC, Anthem XR, Mirai 336BC, Fantastic XR, Triumph, Mirai 301BC, Stellar, American Dream, Mirai 350BC, and Obsession.

### Flint Corn

Flint corn varieties include Bronze-Orange, Candy Red Flint, Floriani Red Flint, Glass Gem, Indian Ornamental (Rainbow), Mandan Red Flour, Painted Mountain, Petmecky, Cherokee White Flour,

### Popcorn

Popcorn varieties include Monarch Butterfly, Yellow Butterfly, Midnight Blue, Ruby Red, Mixed Baby Rice, Queen Mauve, Mushroom Flake, Japanese Hull-less, Strawberry, Blue Shaman, Miniature Colored, Miniature Pink, Pennsylvania Dutch Butter Flavor, and Red Strawberry.

### Dent Corn

Dent corn varieties include Bloody Butcher, Blue Clarage, Ohio Blue Clarage, Cherokee White Eagle, Hickory Cane, Hickory King, Jellicorse Twin, Kentucky Rainbow, Daymon Morgan's Knt. Butcher, Leaming, Leaming's Yellow, McCormack's Blue Giant, Neal Paymaster, Pungo Creek Butcher, Reid's Yellow Dent, Rotten Clarage, and Tennessee Red Cob.

In some embodiments, corn varieties include P1618W, P1306W, P1345, P1151, P1197, P0574, P0589, and P0157. W=white corn.

In some embodiments, the compositions, methods, kits, and systems described herein are suitable for any hybrid of the maize varieties set forth herein.

The compositions, methods, kits, and systems described herein are suitable for any of a hybrid, variety, lineage, etc. of genetically modified maize plants or part thereof. Furthermore, the compositions, methods, kits, and systems described herein are suitable for any of the following genetically modified maize events, which have been approved in one or more countries, or any new genetically modified corn event, which may include Bt traits, glufosinate resistance, glyphosate resistance, etc.: 32138 (32138 SPT Maintainer), 3272 (ENOGEN), 3272x Bt11, 3272 x bt11 x GA21, 3272 x Bt11 x MIR604, 3272 x Bt11 x MIR604 x GA21, 3272 x Bt11 x MIR604 x TC1507 x 5307 x GA21, 3272 x GA21, 3272 x MIR604, 3272 x MIR604 x GA21, 4114, 5307 (AGRISURE Duracade), 5307 x GA21, 5307 x MIR604 x Bt11 x TC1507x GA21 (AGRISURE Duracade 5122), 5307 x MIR604 x Bt11 x TC1507 x GA21 x MIR162 (AGRISURE Duracade 5222), 59122 (Herculex RW), 59122 x DAS40278, 59122 x GA21, 59122 x MIR604, 59122 x MIR604 x GA21, 59122 x MIR604 x TC1507, 59122 x MIR604 x TC1507 x GA21, 59122 x MON810, 59122 x MON810 x MIR604, 59122 x MON810 x NK603, 59122 x MON810 x NK603 x MIR604, 59122 x MON88017, 59122 x MON88017 x DAS40278, 59122 x NK603 (Herculex RW ROUNDUP READY 2), 59122 x NK603 x MIR604, 59122 x TC1507 x GA21, 676, 678, 680, 3751 IR, 98140, 98140 x 59122, 98140 x TC1507, 98140 x TC1507 x 59122, Bt10 (Bt10), Bt11 [X4334CBR, X4734CBR] (AGRISURE CB/LL), Bt11 x 5307, Bt11 x 5307 x GA21, Bt11 x 59122 x MIR604, Br11 x 59122 x MIR604x GA21, Bt11 x 59122 x MIR604 x TC1507, M53, M56, DAS-59122-7, Bt11 x 59122 x MIR604 x TC1507 x GA21, Bt11 x 59122 x TC1507, TC1507 x DAS-59122-7, Bt11 x 59122x TC1507 x GA21, Bt11 x GA21 (AGRISURE GT/CB/LL), Bt11 x MIR162 (AGRISURE Viptera 2100), BT11 x MIR162 x 5307, Bt11 x MIR162 x 5307 x GA21, Bt11 x MIR162 x GA21 (AGRISURE Viptera 3110), Bt11 x MIR162 x MIR604 (AGRISURE Viptera 3100), Bt11 x MIR162 x MIR604 x 5307, Bt11 x MIR162 x MIR604 x 5307 x GA21, Bt11 x MIR162x MIR604 x GA21 (AGRISURE Viptera 3111/AGRISURE Viptera 4), Bt11, MIR162 x MIR604 x MON89034 x 5307 x GA21, Bt11 x MIR162 x MIR604 x TC1507, Bt11 x MIR 162x MIR604 x TC1507 x 5307, Bt11 x MIR162 x MIR604 x TC1507 x GA21, Bt11 x MIR162x MON89034, Bt11 x MIR162 x MON89034 x GA21, Bt11 x MIR162 x TC1507, Bt11 x MIR162 x TC1507 x 5307, Bt11 x MIR162 x TC1507 x 5307 x GA21, Bt11 x MR162 x TC1507 x GA21 (AGRISURE Viptera 3220), BT11 x MIR604 (Agrisure BC/LL/RW), Bt11x MIR604 x 5307, Bt11 x MIR604 x 5307 x GA21, Bt11 x MIR604 x GA21, Bt11 x MIR604x TC1507, Bt11 x MIR604 x TC1507 x 5307, Bt11 x MIR604 x TC1507 x GA21, Bt11 x MON89034 x GA21, Bt11 x TC1507, Bt11 x TC1507 x 5307, Bt11 x TC1507 x GA21, Bt176 (NaturGard KnockOut/Maximizer), BVLA430101, CBH-351 (STARLINK Maize), DAS40278 (ENLIST Maize), DAS40278 x NK603, DBT418 (Bt Xtra Maize), DLL25 [B16], GA21 (ROUNDUP READY Maize/AGRISURE GT), GA21 x MON810 (ROUNDUP READY Yieldgard Maize), GA21 x T25, HCEM485, LY038 (MAVERA Maize), LY038 x MON810 (MAVERA Yieldgard Maize), MIR162 (AGRISURE Viptera), MIR162 x 5307, MIR162 x 5307 x GA21, MIR162 x GA21, MIR162 x MIR604, MIR162 x MIR604 x 5307, MIR 162 x MIR604 x 5307 x GA21, MIR 162 x MIR604 x GA21, MIR 162 x MIR604 x TC1507 x 5307, MIR162 x MIR604 x TC1507 x 5307 x GA21, MIR162 x MIR604 x TC1507 x GA21, MIR162 x MON89034, MIR162 x NK603, MIR162 x TC1507, MIR162 x TC1507 x 5307, MIR 162 x TC1507 x 5307 x GA21, MIR162 x TC1507 x GA21, MIR604 (AGRISURE RW), MIR604 x 5307, MIR604 x 5307 x GA21, MIR604 x GA21 (AGRISURE GT/RW), MIR604x NK603, MIR604 x TC1507, MIR604 x TC1507 x 5307, MIR604 x TC1507 x 5307 xGA21, MIR604 x TC1507 x GA21, MON801 [MON80100], MON802, MON809, MON810 (YIELDGARD, MAIZEGARD), MON810 x MIR162, MON810 x MIR162 x NK603, MON810 x MIR604, MON810 x MON88017 (YIELDGARD VT Triple), MON810 x NK603x MIR604, MON832 (ROUNDUP READY Maize), MON863 (YIELDGARD Rootworm RW, MAXGARD), MON863 x MON810 (YIELDGARD Plus), MON863 x MON810 x NK603 (YIELDGARD Plus with RR), MON863 x NK603 (YIELDGARD RW+RR), MON87403, MON87411, MON87419, MON87427 (ROUNDUP READY Maize), MON87427 x 59122, MON87427 x MON88017, MON87427 x MON88017 x 59122, MON87427 x MON89034, MON87427 x MON89034 x 59122, MON87427 x MON89034 x MIR162 x MON87411, MON87427 x MON89034 x MON88017, MON87427 x MON89034 x MON88017 x 59122, MON87427 x MON89034 x NK603, MON87427 x MON89034 x TC1507, MON87427 x MON89034 x TC1507 x 59122, MON87427 x MON89034 x TC1507 x MON87411 x 59122, MON87427 x MON89034 x TC1507 x MON87411 x 59122 x DAS40278, MON87427 x MON89034 x TC1507 x MON88017, MON87427 x MON89034 x MIR162 x NK603, MON87427 x MON89034 x TC1507 x MON88017 x 59122, MON87427 x TC1507, MON87427 x TC1507 x 59122, MON87427 x TC1507 x MON88017, MON87427 x TC1507x MON88017 x 59122, MON87460 (GENUITY DROUGHTGARD), MON87460 x MON88017, MON87460 x MON89034 x MON88017, MON87460 x MON89034 x NK603, MON87460 x NK603, MON88017, MON88017 x DAS40278, MON89034, MON89034 x 59122, MON89034 x 59122 x DAS40278, MON89034 x 59122 x MON88017, MON89034 x 59122 x MON88017 x DAS40278, MON89034 x DAS40278, MON89034 x MON87460, MON89034 x MON88017 (GENUITY VT Triple Pro), MON89034 x MON88017 x DAS40278, MON89034 x NK603 (GENUITY VT Double Pro), MON89034 x NK603 x DAS40278, MON89034 x TC1507, MON89034 x TC1507 x 59122, MON89034 x TC1507 x 59122 x DAS40278, MON89034 x TC1507 x DAS40278, MON89034 x TC1507 x MON88017, MON89034 x TC1507 x MON88017 x 59122 (GENUITY SMARTSTAX), MON89034 x TC1507 x MON88017 x 59122 x DAS40278, MON89034 x TC1507 x MON88017 x DAS40278, MON89034 x TC1507 x NK603 (POWER CORE), MON89034 x TC1507 x NK603 x DAS40278, MON89034 x TC1507 x NK603 x MIR162, MON89034 x TC1507 x NK603 x MIR162 x DAS40278, MON89034 x GA21, MS3 (INVIGOR Maize), MS6 (INVIGOR Maize), MZHGOJG, MZIR098, NK603 (ROUNDUP READY 2 Maize), NK603 x MON810 x 4114 x MIR604, NK603 x MON810 (YIELDGARD CB+RR), NK603x T25 (ROUNDUP READY LIBERTY LINK Maize), T14 (LIBERTY LINK Maize), T25 (LIBERTY LINK Maize), T25 x MON810 (LIBERTY LINK YIELDGARD Maize), TC1507 (HERCULEX I, HERCULEX CB), TC1507 x 59122 x MON810 x MIR604 x NK603 (OPTIMUM INTRASECT XTREME), TC1507 x MON810 x MIR604 x NK603, TC1507 x 5307, TC1507 x 5307 x GA21, TC1507 x 59122 (HERCULEX XTRA), TC1507 x 59122 x DAS40278, TC1507 x 59122 x MON810, TC1507 x 59122 x MON810 x MIR604, TC1507 x 59122 x MON810 x NK603 (OPTIMUM INTRASECT XTRA), TC1507 x 59122 x MON88017, TC1507 x 59122 x MON88017 x DAS40278, TC1507 x 59122 x NK603 (HERCULEX XTRA RR), TC1507 x 59122 x NK603 x MIR604, TC1507 x DAS40278, TC1507 x GA21, TC1507 x MIR162 x NK603, TC1507 x MIR604 x NK603 (OPTIMUM TRISECT), TC1507 x MON810, TC1507 x MON810 x MIR162, TC1507 x MON810 x MIR162 x NK603, TC1507 x MON810 x MIR604, TC1507 x MON810 x NK603 (OPTIMUM INTRASECT), TC1507 x MON810 x NK603 x MIR604, TC1507 x MON88017, TC1507 x MON88017 x DAS40278, TC1507 x NK603 (HERCULEX I RR), TC1507 x NK603 x DAS40278, TC6275, and VCO-01981-5.

In some embodiments, the present disclosure relates to a plant treated with the composition, kits, systems, and methods disclosed herein. In some embodiments, the plant is a corn seed.

### Budapest Treaty on the International Recognition of the Deposit of Microorganisms for the Purpose of Patent Procedures

The microbial deposits of the present disclosure were made under the provisions of the Budapest Treaty on the International Recognition of the Deposit of Microorganisms for the Purpose of Patent Procedure (Budapest Treaty).

Applicants state that pursuant to 37 C.F.R. § 1.808 (a) (2) “all restrictions imposed by the depositor on the availability to the public of the deposited material will be irrevocably removed upon the granting of the patent.” This statement is subject to paragraph (b) of this section (i.e. 37 C.F.R. § 1.808 (b)).

The Enterobacter sacchari has now been reclassified as Kosakonia sacchari, the name for the organism may be used interchangeably throughout the present disclosure.

Some microbes of the present disclosure are derived from two wild-type strains. Strain CI006 is a bacterial species previously classified in the genus Enterobacter (see aforementioned reclassification into Kosakonia). Strain CI019 is a bacterial species classified in the genus Rahnella. The deposit information for the CI006 Kosakonia wild type (WT) and CI019 Rahnella WT are found in Table 4.

Some microorganisms described in this application were deposited on Jan. 6, 2017 or Aug. 11, 2017 with the Bigelow National Center for Marine Algae and Microbiota (NCMA), located at 60 Bigelow Drive, East Boothbay, Maine 04544, USA. As aforementioned, all deposits were made under the terms of the Budapest Treaty on the International Recognition of the Deposit of Microorganisms for the Purposes of Patent Procedure. The Bigelow National Center for Marine Algae and Microbiota accession numbers and dates of deposit for the aforementioned Budapest Treaty deposits are provided in Table 4.

Biologically pure cultures of Kosakonia sacchari (WT), Rahnella aquatilis (WT), and a variant/remodeled Kosakonia sacchari strain were deposited on Jan. 6, 2017 with the Bigelow National Center for Marine Algae and Microbiota (NCMA), located at 60 Bigelow Drive, East Boothbay, Maine 04544, USA, and assigned NCMA Patent Deposit Designation numbers 201701001, 201701003, and 201701002, respectively. The applicable deposit information is found in Table 4.

Biologically pure cultures of variant/remodeled Kosakonia sacchari strains were deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation numbers 201708004, 201708003, and 201708002, respectively. The applicable deposit information is found in Table 4.

A biologically pure culture of Klebsiella variicola (WT) was deposited on Aug. 11, 2017 with the Bigelow National Center for Marine Algae and Microbiota (NCMA), located at 60 Bigelow Drive, East Boothbay, Maine 04544, USA, and assigned NCMA Patent Deposit Designation number 201708001. Biologically pure cultures of two Klebsiella variicola variants/remodeled strains were deposited on Dec. 20, 2017 with the Bigelow National Center for Marine Algae and Microbiota (NCMA), located at 60 Bigelow Drive, East Boothbay, Maine 04544, USA, and assigned NCMA Patent Deposit Designation numbers 201712001 and 201712002, respectively. The applicable deposit information is found in Table 4.

Biologically pure cultures of two Kosakonia sacchari variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126575 and PTA-126576. Biologically pure cultures of four Klebsiella variicola variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126577, PTA-126578, PTA-126579 and PTA-126580. A biologically pure culture of a Paenibacillus polymyxa (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126581. A biologically pure culture of a Paraburkholderia tropica (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126582. A biologically pure culture of a Herbaspirillum aquaticum (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126583. Biologically pure cultures of four Metakosakonia intestini variants/remodeled strains were deposited on Dec. 23, 2019 with the American Type Culture Collection (ATCC), located at 10801 University Boulevard, Manassas, Virginia 20110-2209, USA and assigned ATCC Patent Deposit Numbers PTA-126584, PTA-126586, PTA-126587 and PTA-126588. A biologically pure culture of a Metakosakonia intestini (WT) strain was deposited on Dec. 23, 2019 with the American Type Culture Collection (ATCC), located at 10801 University Boulevard, Manassas, Virginia 20110-2209, USA and assigned ATCC Patent Deposit Number PTA-126585. A biologically pure culture of a Klebsiella variicola variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126740. A biologically pure culture of a Kosakonia sacchari variant/remodeled strain was deposited on Mar. 25, 2020 with the American Type Culture Collection (ATCC), located at 10801 University Boulevard, Manassas, Virginia 20110-2209, USA and assigned ATCC Patent Deposit Number PTA-126743. The applicable deposit information is found in Table 4.

### EXAMPLES

### Example 1: Generation of Non-Intergeneric Remodeled Bacteria Strain Deposited as PTA-126743

Kosakonia sacchari strain PTA-126743 was generated using the suicide plasmid mutagenesis method, and comprises a partial deletion of nifL; the initial 30 bp and last 83 bp were kept. The native Prm5 promoter (313 bp) was inserted in place of the deleted section of nifL. It further comprises complete deletion of the glnD gene (2,676 bp), including start and stop codons, and Partial deletion of glnE gene (first 1287 bp).

In nitrogen-fixing bacteria, the nif regulon includes the genes that encode the regulatory proteins and the catalytic enzymes in the nitrogen-fixation pathway. NifA is an integral regulator of the nif regulon and is encoded by the gene nifA, which is located immediately downstream of the gene nifL. NifL is an inhibitor that primarily acts in nitrogen-rich conditions to disrupt the activity of NifA, thus decreasing the nitrogenase activity.

Loss of NifL function consequently removes this repression and results in increased NifA activity through this derepression. Since the genetic sequences of nifL and nifA overlap, nifL cannot be fully deleted. Instead, the gene was disrupted by retaining the first 30 base pairs and the last 83 base pairs, removing the middle region of the nifL coding sequence, and inserting a promoter (Prm5). Prm5 is the promoter that natively drives ompX and was found to be a strong and constitutive promoter based on RNA expression level studies done (data not shown). Prm5 is naturally present elsewhere in the genome of the wild-type strain. With this design, Prm5 drives expression of NifA constitutively to increase its activity.

The glnD gene encodes a bifunctional enzyme that can uridylate and deuridylate down-stream signaling proteins based on cell's nitrogen status. When nitrogen is limited, GlnD uridylates PII signaling protein GlnB, which activates the autophosphorylation of NtrB, and subsequently the phosphorylation of the transcriptional regulator NtrC3. Phosphorylated NtrC activates the transcription of the nifLA operon along with more than 30 genes that play role in nitrogen fixation and assimilation. While NifA activates the transcription of the nif regulon, NifL binds NifA and keeps its activity under control. This inhibitory interaction between NifL and NifA is disrupted under nitrogen limited conditions by the uridylated PII protein GlnK, allowing NifA to activate the transcription of the nif regulon.

Aside from activating the nitrogen fixation genes, uridylated PII proteins also activate the assimilation of the reduced nitrogen in the form of glutamine by modulating the activity of the GlnE enzyme. GlnE is a bifunctional enzyme that modulates the activity of the glutamine synthetase (GlnA) through adenylation and deadenylation depending on the nitrogen status of the cells. When nitrogen is limited, the uridylated PII protein GlnB activates GlnA activity through the deadenylase activity of GlnE. When sufficient levels of glutamine are reached, GlnD deuridylates the PII proteins, which in turn inhibits ammonia assimilation through GlnA by activating the adenylase activity of GlnE. Deuridylated PII proteins also down-regulate the NifA activity both at the transcriptional and post-translational levels, leading to a quick turn-off of the nitrogen fixation and assimilation pathways.

To remove the glnD dependent nitrogen sensing pathways, the coding sequence of glnD gene was deleted by removing all of the 2,676 nucleotides, including start and stop codons. Because rest of the protein remains unchanged, no new protein coding sequence is added, and no frame-shift mutations have occurred, this strain contains no novel protein sequences and is unlikely to introduce a non-native enzymatic activity to the cell.

The glnE gene encodes an enzyme, known as glutamine synthetase adenylyl transferase or glutamine-ammonia-ligase adenylyl transferase, that regulates the activity of glutamine synthetase (GS) in response to intracellular levels of glutamine. The GlnE protein consists of two domains with independent and distinct enzymatic activities: an adenylyltransferase (ATase) domain, which covalently modifies the GS protein with an adenylyl group, thus reducing GS activity; and an adenylyl-removing (AR) domain, which removes the adenylyl group from GS, thus increasing its activity. The first 1287 bp following the ATG start codon of the glnE gene were deleted, resulting in a GlnE protein lacking the AR domain but functionally expressing the ATase domain. The remaining sequence of the glnE gene and surrounding sequences remain unchanged. Because the ATase domain remains unchanged, no new protein coding sequence is added, and no frame-shift mutations have occurred, this strain contains no novel protein sequences and is unlikely to introduce a non-native enzymatic activity to the cell.

### Example 2: Generation of Non-Intergeneric Remodeled Bacteria Strain Deposited as PTA-126740

Klebsiella variicola strain PTA-126740 was generated using the suicide plasmid mutagenesis method, and comprises partial deletion of nifL gene; the native CI137 promoter (500 bp) is inserted in place of the deleted section of nifL. It further comprises complete deletion of NT glnD-UTase domain (975 nucleotides).

NifA upregulates the nif gene complex and drives nitrogen fixation when there is insufficient fixed nitrogen available to the microbe. NifL inhibits NifA when there is sufficient fixed N available to the microbe. The nifL and nifA genes are present in an operon and are driven by the same promoter upstream of nifL (Dixon and Kahn 2004). In this strain, most of the nifL coding sequence has been deleted and replaced it with a constitutive promoter naturally present elsewhere in the genome of the wild-type strain. The inserted constitutive promoter is a 500 bp non-coding sequence which contains the native promoter for the infC gene, which is highly expressed in nitrogen-replete conditions in the wild-type strain. In this strain, the native promoter of the infC gene has been left intact, while the 500 bp promoter sequence has been duplicated and inserted in the nifL deletion upstream of nifA. This allows NifA to be both expressed and active in nitrogen-replete conditions, such as a fertilized field. In this strain, both the native and inserted promoter can drive nifA transcription; however, in fertilized conditions, the native nifLA operon promoter is not active because it is repressed by exogenous nitrogen, and therefore nifA transcription is driven only by the inserted promoter.

As described above in Example 1, the glnD gene encodes a bifunctional enzyme that can uridylate and deuridylate down-stream signaling proteins based on cell's nitrogen status The GlnD protein has four domains: an N-terminal uridyl-transferase (UTase) domain; a central uridyl-removal (UR) domain, and two C-terminal ACT domains. The UTase activity is localized to the N-terminal NT domain. This domain has a distinct amino acid residue pattern with conserved glycine (G) and aspartate (D) residues that are important for nucleotidyltransferase activity and binding of metal ions respectively. Both activities are essential for substrate catalysis. Most substitutions for conserved glycine and aspartate residues in this domain abolish glnD's UTase activity, preventing this enzyme from activating PII dependent nitrogen fixation and assimilation pathways. To down-regulate nitrogen assimilation pathways, the NT glnD-UTase domain was deleted by removing 975 nucleotides after the start codon. Because rest of the protein remains unchanged, no new protein coding sequence is added, and no frame-shift mutations have occurred, this strain contains no novel protein sequences and is unlikely to introduce a non-native enzymatic activity to the cell.

### Example 3: Application of a Plurality of Non-Intergeneric Remodeled Bacteria Combined with a Biofertilizer Comprising Plant Growth-Promoting Fungi

Non-intergeneric remodeled N fixing bacteria, such as those described herein and above in Examples 1 and 2 can be applied as a seed treatment, or mixed into an application tank, and applied in-furrow at planting. Agricultural biologicals, such as biofertilizers comprising plant-growth promoting fungi can be intermixed in the same application tank, or applied separately, for synergistic plant beneficial effects.

For example, Rootella®X and other mycorrhizal products have been shown to increase root mass and provide “extended root structure” via fungal hyphae. This combination creates greater root volume, which could provide more room for colonization and greater N fixation by the non-intergeneric remodeled N fixing bacteria described herein. This in turn would allow the plants roots to be in the presence of a greater amount of nitrogen and would increase the plants' ability to uptake nitrogen.

Thus, combined application of the non-intergeneric remodeled N fixing bacteria, such as those described in Examples 1 and 2, with mycorrhizal products could lead to greater plant yield, increased whole plant nitrogen, increased resistance to abiotic and biotic stressors, reduced fertilizer requirements, and increased nitrogen uptake and use efficiency.

Examples of other mycorrhizal fungi products include, but are not limited to, BioFlex, EndoFuse, and MycoApply products, such as MycoGold.

### Example 4: Application of a Plurality of Non-Intergeneric Remodeled Bacteria Combined with a Biofertilizer Comprising Plant-Growth Promoting Rhizobacteria

Non-intergeneric remodeled N fixing bacteria, such as those described herein and above in Examples 1 and 2 can be applied as a seed treatment, or mixed into an application tank, and applied in-furrow at planting. Agricultural biologicals, such as biofertilizers comprising plant-growth promoting bacteria can be intermixed in the same application tank, or applied separately, for synergistic plant beneficial effects.

For example, Terrasym 450 comprises Methylobacterium gregans, a species of plant growth-promoting rhizobacteria. It has been shown to speed up germination and increase early season root development. This directly results in the plants ability uptake nutrients throughout the growing season.

With the early season root development, the non-intergeneric remodeled N fixing bacteria, such as those described herein, will be able to quickly colonize the roots as they form. The quicker the root structure develops, the quicker the microbes can feed off of the root exudates and begin to colonize. Speed of germination and quick development of a larger root structure will decrease the amount of time that the non-intergeneric remodeled N fixing bacteria must survive in the soil or on the seed before they are able to feed off the root exudates and begin colonization.

Additionally, the larger early season root structure that has been reported from using Terrasym 450 will provide more root structure to be able to be colonized, and increased nitrogen directly around the roots. This could provide increased nitrogen fixation and enhanced nitrogen uptake in the plant as the roots will be in the presence of a greater amount of fixed nitrogen.

Thus, combined application of the non-intergeneric remodeled N fixing bacteria, such as the lines described in Examples 1 and 2, with methylotrophs could lead to greater plant yield, increased whole plant nitrogen, reduced fertilizer requirements, and biotic and abiotic stresses

Examples of other products comprising methylotrophs include, but are not limited to, Utrisha™.

### Example 5: Application of a Plurality of Non-Intergeneric Remodeled Bacteria Combined with Biostimulants

Non-intergeneric remodeled N fixing bacteria, such as those described herein, and above in Examples 1 and 2 can be applied as a seed treatment, or mixed into an application tank and applied in-furrow. Biostimulants can be applied simultaneously or separately, and/or at a later time point, for synergistic plant beneficial effects.

For example, SOURCE® is a biostimulant that is applied as a foliar spray. It sends a signal to bacteria in the soil which mirrors the plants natural microbe signals. These signals activate bacteria present in the soil and increases their activity. This in turn allows for nitrogen fixing bacteria and phosphorous solubilizing bacteria to provide these nutrients in a greater amount to the plant and make them more available to the plant.

Thus, combined application of the non-intergeneric remodeled N fixing bacteria, such as the lines described in Examples 1 and 2, with biostimulants could lead to greater plant yield, increased whole plant nitrogen, and reduced fertilizer requirements.

Examples of other biostimulant products include, but are not limited to, Pacesetter, Toggle, Accomplish® Max, MaritimeR, Ferticell® Calcium 880 Plus, Ferticell® Microelements 1-0-0, Ferticell® Nutri-Plus 2.5-0-0, Ferticell® Universal 0-0-1, Norkelp, Phyco Terra®, N-Ext RGS™.

### INCORPORATION BY REFERENCE

All references, articles, publications, patents, patent publications, and patent applications cited herein are incorporated by reference in their entireties for all purposes. PCT published applications WO2017/011602, WO2018/132774, WO2020/006064, WO2020/118111, WO2020/014498, WO2020/163251, WO2021/146209, WO2021/222643, WO2022/029661, and WO2022/261433, and U.S. Pat. Nos. 11,678,667, and 11,678,668, are also hereby incorporated by reference in its entirety for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS

1. A composition, comprising:
a) a plurality of engineered bacteria having at least one genetic variation introduced into at least one gene, or non-coding polynucleotide, of the nitrogen fixation or assimilation genetic regulatory network; and
b) an agricultural biological.

2. The composition of embodiment 1, wherein the plurality of engineered bacteria are non-intergeneric remodeled bacteria.

3 The composition of any one of embodiments 1-2, wherein the plurality of engineered bacteria are transgenic bacteria.

4. The composition of any one of embodiments 1-3, wherein the biological is a biostimulant, biopesticide, or biofertilizer.

5. The composition of any one of embodiments 1-4, wherein the biological is a biostimulant.

6. The composition of any one of embodiments 1-4, wherein the biological is a biopesticide.

7. The composition of any one of embodiments 1-4, wherein the biological is a biofertilizer.

8 The composition of any one of embodiments 1-4, wherein the biological is a biostimulant that comprises humic substances, hormones, cell signaling molecules, seaweed extract, and/or amino acids.

9 The composition of any one of embodiments 1-4, wherein the biological is biopesticide selected from the group consisting of a biochemical pesticide, a microbial pesticide, and a plant-incorporated-protectants pesticide.

10. The composition of any one of embodiments 1-4, wherein the biological is a biofertilizer selected from the group consisting of a bacterial, algal, and fungal biofertilizer.

11. The composition of any one of embodiments 1-4, wherein the biological is a biofertilizer that comprises at least one of a nitrogen fixer, a phosphate solubilizer, a nutrient mobilizer, plant growth-promoting bacteria, and plant growth-regulating bacteria.

12. The composition of any one of embodiments 1-11, wherein the agricultural biological comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

13. The composition of any one of embodiments 1-11, wherein the agricultural biological is a mycorrhizal fungi selected from Glomus intraradices, Glomus mosseae, Glomus aggregatum, Glomus etunicatum, Glomus clarus, and Rhizophagus intraradices.

14. The composition of any one of embodiments 1-11, wherein the agricultural biological is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

15. The composition of any one of embodiments 1-11, wherein the agricultural biological is a species of Gluconacetobacter selected from G. azotocaptans, G. diazotrophicus, G. johannae, and G. sacchari.

16. The composition of any one of embodiments 1-15, wherein the agricultural biological comprises Azotobacter vinelandii and Clostridium pasteurianum.

17. The composition of any one of embodiments 1-16, wherein the agricultural biological comprises a mycorrhizal fungi, Beauveria bassiana, Azospirillum sp., Azotobacter sp., and Rhodopseudomonas palustris.

18. The composition of any one of embodiments 1-17, wherein the agricultural biological comprises a species of Bacillus selected from B. subtilis, B. simplex, B. methylotrophicus, B. amyloliquefaciens, B. megaterium, and B. licheniformis.

19. The composition of any one of embodiments 1-18, wherein the agricultural biological comprises a mycorrhizal fungi, Clostridium sp., and Azotobacter sp.

20. The composition of any one of embodiments 1-19, wherein the agricultural biological comprises Bacillus amyloliquefaciens and Trichoderma virens.

21. The composition of any one of embodiments 1-20, wherein the agricultural biological comprises a species of Trichoderma selected from T. harzianum, T. atroviride, T. asperellum, and T. hamatum.

22. The composition of any one of embodiments 1-21, wherein the agricultural biological comprises Ochrobactrum anthropic, Bacillus Subtillus, and Bacillus simplex.

23. The composition of any one of embodiments 1-22, wherein the agricultural biological comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

24. The composition of any one of embodiments 1-23, wherein the agricultural biological comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii

25. The composition of any one of embodiments 1-24, wherein the agricultural biological comprises a seaweed extract from Reynoutria sachalinensis and/or Ascophyllum nodosum.

26. The composition of any one of embodiments 1-25, wherein the biological is a biostimulant comprising a compound of Formula (2) or a salt, solvent, or isomer thereof, wherein Formula (2) comprises:

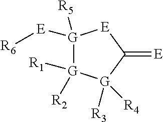

each E is independently O, S, or —NR₇;
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈,

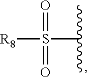

or a lone electron pair, wherein

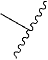

indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and

R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

27. The composition of embodiment 24, wherein
each E is independently O,
each G is independently C,
R₁ and R₅, are each independently H,
R₂ and R₃ together form a bond
R₄ is substituted or unsubstituted alkyl, and
R₆ is substituted or unsubstituted heterocycloalkyl.

28. The composition of any one of embodiments 1-27, wherein the biological is a biostimulant comprising a compound of Formula (3), wherein Formula (3) comprises:

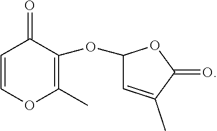 [SMILES: CC1=CC(Oc2c(C)occc2=O)OC1=O]

29. The composition of any one of embodiments 1-28, wherein the biological is a biostimulant comprising a maltol compound.

30. The composition of any one of embodiments 1-29, wherein the biological is a biostimulant comprising a lactone compound.

31. The composition of any one of embodiments 1-30, wherein the biological is a biostimulant comprising a maltol lactone compound.

32. The composition of any one of embodiments 1-31, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

33. The composition of any one of embodiments 1-32, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

34. The composition of any one of embodiments 1-33, wherein the engineered bacteria comprise at least one microbial species capable of fixing atmospheric nitrogen in the presence of exogenous nitrogen.

35. The composition of any one of embodiments 1-34, wherein the engineered bacteria comprise an engineered diazotroph having increased nitrogen fixation activity as compared to an unmodified organism of the same species as said engineered, diazotroph, and wherein the genetic variation comprises genetic material that originates from at least one organism of the same species as said engineered diazotroph.

36. The composition of any one of embodiments 1-35, wherein the engineered bacteria comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

37. The composition of any one of embodiments 1-36, wherein the engineered bacteria comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

38. The composition of any one of embodiments 1-37, wherein the engineered bacteria do not comprise genetic material that originates from a different species than the remodeled bacteria.

39. The composition of any one of embodiments 1-38, wherein the engineered bacteria comprise an epiphyte.

40. The composition of any one of embodiments 1-39, wherein the engineered bacteria comprise an endophyte.

41. The composition of any one of embodiments 1-40, wherein the engineered bacteria comprise a rhizophyte.

42. The composition of any one of embodiments 1-41, wherein the engineered bacteria comprise at least one microbial species that is a non-intergeneric remodeled microbial species comprising an introduced control sequence operably linked to at least one gene of the nitrogen fixation or assimilation genetic regulatory network.

43. The composition of any one of embodiments 1-42, wherein the engineered bacteria comprise at least one microbial species that is a non-intergeneric remodeled microbial species comprising a heterologous promoter operably linked to at least one gene of the nitrogen fixation or assimilation genetic regulatory network.

44. The composition of any one of embodiments 1-43, wherein the engineered bacteria comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

45. The composition of any one of embodiments 1-44, wherein the engineered bacteria comprise at least one microbial species that is a non-intergeneric remodeled microbial species having a mutated nifL gene that has been altered to comprise a heterologous promoter inserted into said nifL gene.

46. The composition of any one of embodiments 1-45, wherein the engineered bacteria comprise at least one microbial species that is a non-intergeneric remodeled microbial species comprising a mutated glnE gene that results in a truncated GlnE protein lacking an adenylyl-removing (AR) domain.

47. The composition of any one of embodiments 1-46, wherein the engineered bacteria comprise at least one microbial species that is a non-intergeneric remodeled microbial species comprising a mutated amtB gene that results in the lack of expression of said amtB gene.

48. The composition of any one of embodiments 1-47, wherein the engineered bacteria comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

49. The composition of any one of embodiments 1-48, wherein the engineered bacteria comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

50. The composition of any one of embodiments 1-49, wherein the engineered bacteria comprise Kosakonia sacchari.

51. The composition of any one of embodiments 1-50, wherein the engineered bacteria comprise Kosakonia sacchari PTA-126743.

52. The composition of any one of embodiments 1-51, wherein the engineered bacteria

53. The composition of any one of embodiments 1-52, wherein the engineered bacteria comprise Klebsiella variicola PTA-126740.

54. The composition of any one of embodiments 1-53, wherein the engineered bacteria comprise Klebsiella variicola PTA-126740 and Kosakonia sacchari PTA-126743.

55. The composition of any one of embodiments 1-54, wherein the composition comprises the engineered bacteria in a liquid formulation.

56. The composition of any one of embodiments 1-55, wherein the plurality of engineered bacteria are reconstituted from a previous powder formulation.

57. The composition of any one of embodiments 1-56, wherein the engineered bacteria are at a concentration of between about 1.0×10⁴ and about 1.0×10¹² CFU/mL of the total volume of the composition.

58. The composition of any one of embodiments 1-57, wherein the composition comprises the engineered bacteria in a powder formulation of lyophilized microbes.

59. The composition of embodiment 58, wherein the composition is encapsulated within a water-soluble package.

60. The composition of any one of embodiments 1-59, further comprising at least one of a polymer, buffer, sugar, sugar alcohol, stabilizer, bulking agent, anti-caking agent, dispersant, biofilm, and isolated biofilm composition.

61. The composition of any one of embodiments 1-60, further comprising a sugar or sugar alcohol.

62. The composition of embodiment 61, wherein the sugar or sugar alcohol is selected from sucrose, lactose, trehalose, sorbitol, mannitol, galactitol, fucitol, iditol, inositol, and combinations thereof.

63. The composition of any one of embodiments 1-62, further comprising a buffer.

64. The composition of embodiment 63, wherein the buffer is selected from potassium phosphate, dipotassium phosphate, monopotassium phosphate, and combinations thereof.

65. The composition of any one of embodiments 1-64, further comprising a polymer.

66. The composition of embodiment 65, wherein the polymer is selected from polyvinylpyrrolidone (PVP), polyvinylpyrrolidone-vinyl acetate (PVP-VA), carboxymethyl cellulose (CMC), hydroxypropyl methylcellulose, alginate, and combinations thereof.

67. The composition of any one of embodiments 1-66, wherein the composition further comprises an agriculturally acceptable adjuvant, excipient, or carrier.

68. A method of treating a plant, comprising: applying the composition of any one of embodiments 1-67 to a plant or plant part or to an area in which a plant will be grown or is growing.

69. The method of embodiment 68, wherein the composition is applied in-furrow, as a seed treatment, as a seedling root dip, as a broadcast, or as a foliar spray.

70. The method of embodiment 68 or 69, wherein the plant is a species of Hordeum, Oryza, Zea, Sorghum, Brassica, or Triticeae.

71. The method of embodiment 68 or 69, wherein the plant is corn.

72. The method of embodiment 68 or 69, wherein the plant is soybean.

73. The method of embodiment 68 or 69, wherein the plant is rice.

74. The method of embodiment 68 or 69, wherein the plant is wheat.

75. The method of embodiment 68 or 69, wherein the plant is rapeseed.

76. The method of embodiment 68 or 69, wherein the plant is sweet corn, flint corn, popcorn, dent corn, pod corn, or flour corn.

77. The method of any one of embodiments 68-76, wherein the method increases plant nutrient uptake compared to an untreated plant.

78. The method of any one of embodiments 68-77, wherein the method increases plant available nitrogen, phosphorus, or potassium compared to an untreated plant.

79. The method of any one of embodiments 68-78, wherein the area is a field, and wherein the non-intergeneric remodeled bacteria have increased nitrogen fixation activity in the field as compared to un-remodeled bacteria of the same species, in the same field.

80. The method of embodiment 79, wherein the field has a fixed nitrogen concentration of at least 0.01 mM.

81. The method of embodiment 79, wherein the field has a fixed nitrogen concentration of at least 0.1 mM. 82. The method of embodiment 79, wherein the field has a fixed nitrogen concentration of at least 0.5 mM.

83. The method of any one of embodiments 68-82, wherein the method decreases the amount of fertilizer required compared to an untreated plant.

84. The method of any one of embodiments 68-83, wherein the method increases plant yield compared to an untreated plant.

85. The method of any one of embodiments 68-84, wherein the method increases chlorophyll content compared to an untreated plant.

86. The method of any one of embodiments 68-85, wherein the method increases resistance to an abiotic or biotic stressor.

87. The method of embodiment 86, wherein the abiotic stressor is low or high temperature, deficient or excessive water, high salinity, heavy metals, or ultraviolet radiation.

88. A method of treating a plant, comprising:
applying a plurality of engineered bacteria having at least one genetic variation introduced into at least one gene, or non-coding polynucleotide, of the nitrogen fixation or assimilation genetic regulatory network to a plant or to an area in which a plant will be grown or is growing; and
applying an agricultural biological to the plant or to an area in which a plant will be grown or is growing.

89. The method of embodiment 88, wherein the plurality of engineered bacteria are non-intergeneric remodeled bacteria.

90. The method of embodiment 88 or 89, wherein the plurality of engineered bacteria are transgenic bacteria.

91. The method of any one of embodiments 88-90, wherein the biological is a biostimulant, biopesticide, or biofertilizer.

92. The method of any one of embodiments 88-91, wherein the biological is a biostimulant.

93. The method of any one of embodiments 88-91, wherein the biological is a biopesticide.

94. The method of any one of embodiments 88-91, wherein the biological is a biofertilizer.

95. The method of any one of embodiments 88-91, wherein the biological is a biostimulant that comprises humic substances, hormones, cell signaling molecules, seaweed extract, and/or amino acids.

96. The method of any one of embodiments 88-91, wherein the biological is biopesticide selected from the group consisting of a biochemical pesticide, a microbial pesticide, and a plant-incorporated-protectants pesticide.

97. The method of any one of embodiments 88-91, wherein the biological is a biofertilizer selected from the group consisting of a bacterial, algal, and fungal biofertilizer.

98. The method of any one of embodiments 88-91, wherein the biological is a biofertilizer that comprises at least one of a nitrogen fixer, a phosphate solubilizer, a nutrient mobilizer, plant growth-promoting bacteria, and plant growth-regulating bacteria.

99. The method of any one of embodiments 88-98, wherein the agricultural biological comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

100. The method of any one of embodiments 88-98, wherein the agricultural biological is a mycorrhizal fungi selected from Glomus intraradices, Glomus mosseae, Glomus aggregatum, Glomus etunicatum, Glomus clarus, and Rhizophagus intraradices.

101. The method of any one of embodiments 88-98, wherein the agricultural biological is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

102. The method of any one of embodiments 88-98, wherein the agricultural biological is a species of Gluconacetobacter selected from G. azotocaptans, G. diazotrophicus, G. johannae, and G. sacchari.

103. The method of any one of embodiments 88-102, wherein the agricultural biological comprises Azotobacter vinelandii and Clostridium pasteurianum.

104. The method of any one of embodiments 88-93, wherein the agricultural biological comprises a mycorrhizal fungi, Beauveria bassiana, Azospirillum sp., Azotobacter sp., and Rhodopseudomonas palustris.

105. The method of any one of embodiments 88-104, wherein the agricultural biological comprises a species of Bacillus selected from B. subtilis, B. simplex, B. methylotrophicus, B. amyloliquefaciens, B. megaterium, and B. licheniformis.

106. The method of any one of embodiments 88-105, wherein the agricultural biological comprises a mycorrhizal fungi, Clostridium sp., and Azotobacter sp.

107. The method of any one of embodiments 88-106, wherein the agricultural biological comprises Bacillus amyloliquefaciens and Trichoderma virens.

108. The method of any one of embodiments 88-107, wherein the agricultural biological comprises a species of Trichoderma selected from T. harzianum, T. atroviride, T. asperellum, and T. hamatum.

109. The method of any one of embodiments 88-108, wherein the agricultural biological comprises Ochrobactrum anthropic, Bacillus Subtillus, and Bacillus simplex.

110. The method of any one of embodiments 88-109, wherein the agricultural biological comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

111. The method of any one of embodiments 88-110, wherein the agricultural biological comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii

112. The method of any one of embodiments 88-111, wherein the agricultural biological comprises a seaweed extract from Reynoutria sachalinensis and/or Ascophyllum nodosum.

113. The method of any one of embodiments 88-112, wherein the biological is a biostimulant comprising a compound of Formula (2) or a salt, solvent, or isomer thereof, wherein Formula (2) comprises:

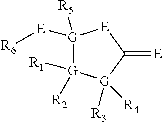

each E is independently O, S, or —NR₇;
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈,

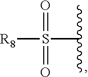

or a lone electron pair, wherein

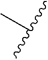

indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and
R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

114. The method of embodiment 113, wherein
each E is independently O,
each G is independently C,
R₁ and R₅, are each independently H,
R₂ and R₃ together form a bond
R₄ is substituted or unsubstituted alkyl, and
R₆ is substituted or unsubstituted heterocycloalkyl.

115. The method of any one of embodiments 88-114, wherein the biological is a biostimulant comprising a compound of Formula (3), wherein Formula (3) comprises:

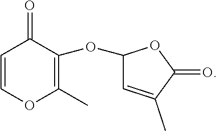 [SMILES: CC1=CC(Oc2c(C)occc2=O)OC1=O]

116. The method of any one of embodiments 88-115, wherein the biological is a biostimulant comprising a maltol compound.

117. The method of any one of embodiments 88-116, wherein the biological is a biostimulant comprising a lactone compound.

118. The method of any one of embodiments 88-117, wherein the biological is a biostimulant comprising a maltol lactone compound.

119. The method of any one of embodiments 88-118, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Kosakonia, Mesorhizobium, Microbacterium, Enterobacter, Klebsiella, Kluyvera, Pseudomonas, Rahnella, Rhizobium, Sinorhizobium, and combinations thereof.

120. The method of any one of embodiments 88-119, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

121. The method of any one of embodiments 88-120, wherein the engineered bacteria comprise at least one microbial species capable of fixing atmospheric nitrogen in the presence of exogenous nitrogen.

122. The method of any one of embodiments 88-121, wherein the engineered bacteria comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

123. The method of any one of embodiments 88-122, wherein the engineered bacteria comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

124. The method of any one of embodiments 88-123, wherein the engineered bacteria comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

125. The method of any one of embodiments 88-124, wherein the engineered bacteria does not comprise genetic material that originates from a different species than the remodeled bacteria.

126. The method of any one of embodiments 88-125, wherein the engineered bacteria comprise an epiphyte.

127. The method of any one of embodiments 88-126, wherein the engineered bacteria comprise an endophyte.

128. The method of any one of embodiments 88-127, wherein the engineered bacteria comprise a rhizophyte.

129. The method of any one of embodiments 88-128, wherein the engineered bacteria comprise at least one microbial species that is a non-intergeneric remodeled microbial species comprising an introduced control sequence operably linked to at least one gene of the nitrogen fixation or assimilation genetic regulatory network.

130. The method of any one of embodiments 88-129, wherein the engineered bacteria comprise at least one microbial species that is a non-intergeneric remodeled microbial species comprising a heterologous promoter operably linked to at least one gene of the nitrogen fixation or assimilation genetic regulatory network.

131. The method of any one of embodiments 88-130, wherein the engineered bacteria comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

132. The method of any one of embodiments 88-131, wherein the engineered bacteria comprise at least one microbial species that is a non-intergeneric remodeled microbial species having a mutated nifL gene that has been altered to comprise a heterologous promoter inserted into said nifL gene.

133. The method of any one of embodiments 88-132, wherein the engineered bacteria comprise at least one microbial species that is a non-intergeneric remodeled microbial species comprising a mutated glnE gene that results in a truncated GlnE protein lacking an adenylyl-removing (AR) domain.

134. The method of any one of embodiments 88-133, wherein the engineered bacteria comprise at least one microbial species that is a non-intergeneric remodeled microbial species comprising a mutated amtB gene that results in the lack of expression of said amtB gene.

135. The method of any one of embodiments 88-134, wherein the engineered bacteria comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

136. The method of any one of embodiments 88-135, wherein the engineered bacteria comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifHI, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

137. The method of any one of embodiments 88-136, wherein the engineered bacteria comprise Kosakonia sacchari.

138. The method of any one of embodiments 88-137, wherein the engineered bacteria comprise Kosakonia sacchari PTA-126743.

139. The method of any one of embodiments 88-138, wherein the engineered bacteria

140. The method of any one of embodiments 88-139, wherein the engineered bacteria comprise Klebsiella variicola PTA-126740.

141. The method of any one of embodiments 88-140, wherein the engineered bacteria comprise Klebsiella variicola PTA-126740 and Kosakonia sacchari PTA-126743.

142. The method of any one of embodiments 88-141, wherein the engineered bacteria are a liquid formulation.

143. The method of any one of embodiments 88-142, wherein the plurality of engineered bacteria are reconstituted from a previous powder formulation.

144. The method of any one of embodiments 88-143, wherein the engineered bacteria are at a concentration of between about 1.0×10⁴ and about 1.0×10¹² CFU/mL of the total volume of the composition.

145. The method of any one of embodiments 88-144, wherein the engineered bacteria are a powder formulation of lyophilized microbes.

146. The method of embodiment 145, wherein the engineered bacteria are encapsulated within a water-soluble package.

147. The method of any one of embodiments 88-146, wherein the method comprises mixing the engineered bacteria and the agricultural biological prior to application to a plant or to an area in which a plant will be grown or is growing.

148. The method of any one of embodiments 88-147, wherein the agricultural biological is applied in-furrow, as a seed treatment, as a seedling root dip, as a broadcast, or as a foliar spray.

149. The method of any one of embodiments 88-148, wherein the engineered bacteria are applied in-furrow, as a seed treatment, as a seedling root dip, as a broadcast, or as a foliar spray.

150. The method of any one of embodiments 88-149, wherein the engineered bacteria and the agricultural biological are applied simultaneously.

151. The method of embodiments 88-149, wherein the engineered bacteria and the agricultural biological are applied sequentially.

152. The method of embodiment 151, wherein application of the engineered bacteria and the agricultural biological are separated by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days.

153. The method of any one of embodiments 88-152, wherein the plant is a species of Hordeum, Oryza, Zea, Sorghum, Brassica, or Triticeae.

154. The method of any one of embodiments 88-152, wherein the plant is corn.

155. The method of any one of embodiments 88-152, wherein the plant is soybean.

156. The method of any one of embodiments 88-152, wherein the plant is rice.

157. The method of any one of embodiments 88-152, wherein the plant is wheat.

158. The method of any one of embodiments 88-152, wherein the plant is rapeseed.

159. The method of any one of embodiments 88-152, wherein the plant is sweet corn, flint corn, popcorn, dent corn, pod corn, or flour corn.

160. The method of any one of embodiments 88-159, wherein the method increases plant nutrient uptake compared to an untreated plant.

161. The method of any one of embodiments 88-160, wherein the method increases plant available nitrogen, phosphorus, or potassium compared to an untreated plant.

162. The method of any one of embodiments 88-161, wherein the method increases whole plant nitrogen compared to an untreated plant.

163. The method of any one of embodiments 88-162, wherein the method decreases the amount of fertilizer required compared to an untreated plant.

164. The method of any one of embodiments 88-163, wherein the method increases plant yield compared to an untreated plant.

165. The method of any one of embodiments 88-164, wherein the method increases chlorophyll content compared to an untreated plant.

166. The method of any one of embodiments 88-165, wherein the method increases resistance to an abiotic or biotic stressor compared to an untreated plant.

167. The method of embodiment 166, wherein the abiotic stressor is low or high temperature, deficient or excessive water, high salinity, heavy metals, or ultraviolet radiation.

168. The method of any one of embodiments 88-167, wherein the method increases plant nutrient uptake compared to a plant treated with nitrogen fixing bacteria without an agricultural biological, or a plant treated with an agricultural biological without nitrogen fixing bacteria.

169. The method of any one of embodiments 88-168, wherein the method increases plant available nitrogen, phosphorus, or potassium compared to a plant treated with nitrogen fixing bacteria without an agricultural biological, or a plant treated with an agricultural biological without nitrogen fixing bacteria.

170. The method of any one of embodiments 88-169, wherein the method increases whole plant nitrogen compared to a plant treated with nitrogen fixing bacteria without an agricultural biological, or a plant treated with an agricultural biological without nitrogen fixing bacteria.

171. The method of any one of embodiments 88-170, wherein the method decreases the amount of fertilizer required compared to a plant treated with nitrogen fixing bacteria without an agricultural biological, or a plant treated with an agricultural biological without nitrogen fixing bacteria.

172. The method of any one of embodiments 88-171, wherein the method increases plant yield compared to a plant treated with nitrogen fixing bacteria without an agricultural biological, or a plant treated with an agricultural biological without nitrogen fixing bacteria.

173. The method of any one of embodiments 88-172, wherein the method increases chlorophyll content compared to a plant treated with nitrogen fixing bacteria without an agricultural biological, or a plant treated with an agricultural biological without nitrogen fixing bacteria.

174. The method of any one of embodiments 88-173, wherein the method increases resistance to an abiotic or biotic stressor compared to a plant treated with nitrogen fixing bacteria without an agricultural biological, or a plant treated with an agricultural biological without nitrogen fixing bacteria.

175. The method of embodiment 174, wherein the abiotic stressor is low or high temperature, deficient or excessive water, high salinity, heavy metals, or ultraviolet radiation.

176. The method of any one of embodiments 88-175, wherein the area is a field, and wherein the engineered bacteria have increased nitrogen fixation activity in the field as compared to un-remodeled bacteria of the same species, in the same field.

177. The method of embodiment 176, wherein the field has a fixed nitrogen concentration of at least 0.01 mM.

178. The method of embodiment 176, wherein the field has a fixed nitrogen concentration of at least 0.1 mM.

179. The method of embodiment 176, wherein the field has a fixed nitrogen concentration of at least 0.5 mM.

180. A kit comprising:
a) a plurality of engineered bacteria having at least one genetic variation introduced into at least one gene, or non-coding polynucleotide, of the nitrogen fixation or assimilation genetic regulatory network; and
b) an agricultural biological.

181. The kit of embodiment 180, wherein the plurality of engineered bacteria are non-intergeneric remodeled bacteria.

182. The kit of embodiment 180 or 181, wherein the plurality of engineered bacteria are transgenic bacteria.

183. The kit of any one of embodiments 180-182, wherein the biological is a biostimulant, biopesticide, or biofertilizer.

184. The kit of any one of embodiments 180-183, wherein the biological is a biostimulant. 185. The kit of any one of embodiments 180-183, wherein the biological is a biopesticide.

186. The kit of any one of embodiments 180-183, wherein the biological is a biofertilizer.

187. The kit of any one of embodiments 180-183, wherein the biological is a biostimulant that comprises humic substances, hormones, cell signaling molecules, seaweed extract, and/or amino acids.

188. The kit of any one of embodiments 180-183, wherein the biological is biopesticide selected from the group consisting of a biochemical pesticide, a microbial pesticide, and a plant-incorporated-protectants pesticide.

189. The kit of any one of embodiments 180-183, wherein the biological is a biofertilizer selected from the group consisting of a bacterial, algal, and fungal biofertilizer.

190. The kit of any one of embodiments 180-183, wherein the biological is a biofertilizer that comprises at least one of a nitrogen fixer, a phosphate solubilizer, a nutrient mobilizer, plant growth-promoting bacteria, and plant growth-regulating bacteria.

191. The kit of any one of embodiments 180-190, wherein the agricultural biological comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

192. The kit of any one of embodiments 180-190, wherein the agricultural biological is a mycorrhizal fungi selected from Glomus intraradices, Glomus mosseae, Glomus aggregatum, Glomus etunicatum, Glomus clarus, and Rhizophagus intraradices.

193. The kit of any one of embodiments 180-190, wherein the agricultural biological is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

194. The kit of any one of embodiments 180-190, wherein the agricultural biological is a species of Gluconacetobacter selected from G. azotocaptans, G. diazotrophicus, G. johannae, and G. sacchari.

195. The kit of any one of embodiments 180-194, wherein the agricultural biological comprises Azotobacter vinelandii and Clostridium pasteurianum.

196. The kit of any one of embodiments 180-195, wherein the agricultural biological comprises a mycorrhizal fungi, Beauveria bassiana, Azospirillum sp., Azotobacter sp., and Rhodopseudomonas palustris.

197. The kit of any one of embodiments 180-196, wherein the agricultural biological comprises a species of Bacillus selected from B. subtilis, B. simplex, B. methylotrophicus, B. amyloliquefaciens, B. megaterium, and B. licheniformis.

198. The kit of any one of embodiments 180-197, wherein the agricultural biological comprises a mycorrhizal fungi, Clostridium sp., and Azotobacter sp.

199. The kit of any one of embodiments 180-198, wherein the agricultural biological comprises Bacillus amyloliquefaciens and Trichoderma virens.

200. The kit of any one of embodiments 180-199, wherein the agricultural biological comprises a species of Trichoderma selected from T. harzianum, T. atroviride, T. asperellum, and T. hamatum. 201. The kit of any one of embodiments 180-200, wherein the agricultural biological comprises Ochrobactrum anthropic, Bacillus Subtillus, and Bacillus simplex.

202. The kit of any one of embodiments 180-201, wherein the agricultural biological comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

203. The kit of any one of embodiments 180-202, wherein the agricultural biological comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii

204. The kit of any one of embodiments 180-203, wherein the agricultural biological comprises a seaweed extract from Reynoutria sachalinensis and/or Ascophyllum nodosum.

205. The kit of any one of embodiments 180-204, wherein the biological is a biostimulant comprising a compound of Formula (2) or a salt, solvent, or isomer thereof, wherein Formula (2) comprises:

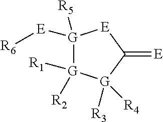

each E is independently O, S, or —NR₇;
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈,

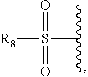

or a lone electron pair, wherein

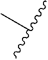

indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and

R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

206. The kit of embodiment 205, wherein
each E is independently O,
each G is independently C,
R₁ and R₅, are each independently H,
R₂ and R₃ together form a bond
R₄ is substituted or unsubstituted alkyl, and
R₆ is substituted or unsubstituted heterocycloalkyl.

207. The kit of any one of embodiments 180-206, wherein the biological is a biostimulant comprising a compound of Formula (3), wherein Formula (3) comprises:

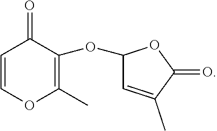 [SMILES: CC1=CC(Oc2c(C)occc2=O)OC1=O]

208. The kit of any one of embodiments 180-207, wherein the biological is a biostimulant comprising a maltol compound.

209. The kit of any one of embodiments 180-208, wherein the biological is a biostimulant comprising a lactone compound.

210. The kit of any one of embodiments 180-209, wherein the biological is a biostimulant comprising a maltol lactone compound.

211. The kit of any one of embodiments 180-210, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Kosakonia, Mesorhizobium, Microbacterium, Enterobacter, Klebsiella, Kluyvera, Pseudomonas, Rahnella, Rhizobium, Sinorhizobium, and combinations thereof.

212. The kit of any one of embodiments 180-211, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

213. The kit of any one of embodiments 180-212, wherein the engineered bacteria comprise at least one microbial species capable of fixing atmospheric nitrogen in the presence of exogenous nitrogen.

214. The kit of any one of embodiments 180-213, wherein the engineered bacteria comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

215. The kit of any one of embodiments 180-214, wherein the engineered bacteria comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

216. The kit of any one of embodiments 180-215, wherein the engineered bacteria comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

217. The kit of any one of embodiments 180-216, wherein the engineered bacteria does not comprise genetic material that originates from a different species than the remodeled bacteria.

218. The kit of any one of embodiments 180-217, wherein the engineered bacteria comprise an epiphyte.

219. The kit of any one of embodiments 180-218, wherein the engineered bacteria comprise an endophyte.

220. The kit of any one of embodiments 180-219, wherein the engineered bacteria comprise a rhizophyte.

221. The kit of any one of embodiments 180-220, wherein the engineered bacteria comprise at least one microbial species that is a non-intergeneric remodeled microbial species comprising an introduced control sequence operably linked to at least one gene of the nitrogen fixation or assimilation genetic regulatory network.

222. The kit of any one of embodiments 180-221, wherein the engineered bacteria comprise at least one microbial species that is a non-intergeneric remodeled microbial species comprising a heterologous promoter operably linked to at least one gene of the nitrogen fixation or assimilation genetic regulatory network.

223. The kit of any one of embodiments 180-222, wherein the engineered bacteria comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

224. The kit of any one of embodiments 180-223, wherein the engineered bacteria comprise at least one microbial species that is a non-intergeneric remodeled microbial species having a mutated nifL gene that has been altered to comprise a heterologous promoter inserted into said nifL gene.

225. The kit of any one of embodiments 180-224, wherein the engineered bacteria comprise at least one microbial species that is a non-intergeneric remodeled microbial species comprising a mutated glnE gene that results in a truncated GlnE protein lacking an adenylyl-removing (AR) domain.

226. The kit of any one of embodiments 180-225, wherein the engineered bacteria comprise at least one microbial species that is a non-intergeneric remodeled microbial species comprising a mutated amtB gene that results in the lack of expression of said amtB gene.

227. The kit of any one of embodiments 180-226, wherein the engineered bacteria comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

228. The kit of any one of embodiments 180-227, wherein the engineered bacteria comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

229. The kit of any one of embodiments 180-228, wherein the engineered bacteria comprise Kosakonia sacchari.

230. The kit of any one of embodiments 180-229, wherein the engineered bacteria comprise Kosakonia sacchari PTA-126743.

231. The kit of any one of embodiments 180-230, wherein the engineered bacteria comprise Klebsiella variicola.

232. The kit of any one of embodiments 180-231, wherein the engineered bacteria comprise Klebsiella variicola PTA-126740.

233. The kit of any one of embodiments 180-232, wherein the engineered bacteria comprise Klebsiella variicola PTA-126740 and Kosakonia sacchari PTA-126743.

234. The kit of any one of embodiments 180-233, wherein the engineered bacteria are a liquid formulation.

235. The kit of any one of embodiments 180-234, wherein the plurality of engineered bacteria are reconstituted from a previous powder formulation.

236. The kit of any one of embodiments 180-235, wherein the engineered bacteria are at a concentration of between about 1.0×10⁴ and about 1.0×10¹² CFU/mL of the total volume of the composition.

237. The kit of any one of embodiments 180-236, wherein the engineered bacteria are a powder formulation of lyophilized microbes.

238. The kit of any one of embodiments 180-237, wherein the engineered bacteria and/or the agricultural biological are encapsulated within a water-soluble package.

239. The kit of any one of embodiments 180-238, wherein the engineered bacteria and/or the agricultural biological comprise at least one of a polymer, buffer, sugar, sugar alcohol, stabilizer, bulking agent, anti-caking agent, dispersant, biofilm, and isolated biofilm composition.

240. The kit of any one of embodiments 180-239, wherein the engineered bacteria and/or the agricultural biological comprise a sugar or sugar alcohol.

241. The kit of embodiment 240, wherein the sugar or sugar alcohol is selected from sucrose, lactose, trehalose, sorbitol, mannitol, galactitol, fucitol, iditol, inositol, and combinations thereof.

242. The kit of any one of embodiments 180-241, wherein the engineered bacteria and/or the agricultural biological comprise a buffer.

243. The kit of embodiment 242, wherein the buffer is selected from potassium phosphate, dipotassium phosphate, monopotassium phosphate, and combinations thereof.

244. The kit of any one of embodiments 180-243, wherein the engineered bacteria and/or the agricultural biological comprise a polymer.

245. The kit of embodiment 244, wherein the polymer is selected from polyvinylpyrrolidone (PVP), polyvinylpyrrolidone-vinyl acetate (PVP-VA), carboxymethyl cellulose (CMC), hydroxypropyl methylcellulose, alginate, and combinations thereof.

246. The kit of any one of embodiments 180-245, wherein the engineered bacteria and/or the agricultural biological comprise an agriculturally acceptable adjuvant, excipient, or carrier.

247. The kit of any one of embodiments 180-246, further comprising instructions for applying the engineered bacteria and/or the agricultural biological to a plant or to an area in which a plant will be grown or is growing.

248. The kit of any one of embodiments 180-247, wherein the plant is corn, soybean, rice, wheat, or rapeseed.

249. A farm administration system, comprising:
a) a plurality of engineered bacteria having at least one genetic variation introduced into at least one gene, or non-coding polynucleotide, of the nitrogen fixation or assimilation genetic regulatory network;
b) an agricultural biological; and
c) instructions for using a) and b) to treat a plant.

250. The farm administration system of embodiment 249, wherein the plurality of engineered bacteria are non-intergeneric remodeled bacteria.

251. The farm administration system of embodiment 249 or 250, wherein the plurality of engineered bacteria are transgenic bacteria.

252. The farm administration system of any one of embodiments 249-251, wherein the biological is a biostimulant, biopesticide, or biofertilizer.

253. The farm administration system of any one of embodiments 249-252, wherein the biological is a biostimulant.

254. The farm administration system of any one of embodiments 249-252, wherein the biological is a biopesticide.

255. The farm administration system of any one of embodiments 249-252, wherein the biological is a biofertilizer.

256. The farm administration system of any one of embodiments 249-252, wherein the biological is a biostimulant that comprises humic substances, hormones, cell signaling molecules, seaweed extract, and/or amino acids.

257. The farm administration system of any one of embodiments 249-252, wherein the biological is biopesticide selected from the group consisting of a biochemical pesticide, a microbial pesticide, and a plant-incorporated-protectants pesticide.

258. The farm administration system of any one of embodiments 249-252, wherein the biological is a biofertilizer selected from the group consisting of a bacterial, algal, and fungal biofertilizer.

259. The farm administration system of any one of embodiments 249-252, wherein the biological is a biofertilizer that comprises at least one of a nitrogen fixer, a phosphate solubilizer, a nutrient mobilizer, plant growth-promoting bacteria, and plant growth-regulating bacteria.

260. The farm administration system of any one of embodiments 249-259, wherein the agricultural biological comprises one or more species of cultured microbe selected from Methylobacterium, mycorrhizal fungi, Gluconacetobacter, Achromobacter, Agrobacterium, Anabaena, Azorhizobium, Azospirillum, Azotobacter, Bacillus, Beauveria, Bradyrhizobium, Kluyvera, Kosakonia, Mesorhizobium, Clostridium, Enterobacter, Klebsiella, Microbacterium, Ochrobactrum, Penicillium, Pseudomonas, Rahnella, Rhizobium, Rhodopseudomonas Sinorhizobium, Trichoderma, and combinations thereof.

261. The farm administration system of any one of embodiments 249-259, wherein the agricultural biological is a mycorrhizal fungi selected from Glomus intraradices, Glomus mosseae, Glomus aggregatum, Glomus etunicatum, Glomus clarus, and Rhizophagus intraradices.

262. The farm administration system of any one of embodiments 249-259, wherein the agricultural biological is a species of Methylobacterium selected from M. gregans, M. adhaesivum, M. aerolatum, M. ajmalii, M. aquaticum M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile.

263. The farm administration system of any one of embodiments 249-259, wherein the agricultural biological is a species of Gluconacetobacter selected from G. azotocaptans, G. diazotrophicus, G. johannae, and G. sacchari.

264. The farm administration system of any one of embodiments 249-263, wherein the agricultural biological comprises Azotobacter vinelandii and Clostridium pasteurianum.

265. The farm administration system of any one of embodiments 249-264, wherein the agricultural biological comprises a mycorrhizal fungi, Beauveria bassiana, Azospirillum sp., Azotobacter sp., and Rhodopseudomonas palustris.

266. The farm administration system of any one of embodiments 249-265, wherein the agricultural biological comprises a species of Bacillus selected from B. subtilis, B. simplex, B. methylotrophicus, B. amyloliquefaciens, B. megaterium, and B. licheniformis.

267. The farm administration system of any one of embodiments 249-266, wherein the agricultural biological comprises a mycorrhizal fungi, Clostridium sp., and Azotobacter sp.

268. The farm administration system of any one of embodiments 249-267, wherein the agricultural biological comprises Bacillus amyloliquefaciens and Trichoderma virens.

269. The farm administration system of any one of embodiments 249-268, wherein the agricultural biological comprises a species of Trichoderma selected from T. harzianum, T. atroviride, T. asperellum, and T. hamatum.

270. The farm administration system of any one of embodiments 249-269, wherein the agricultural biological comprises Ochrobactrum anthropic, Bacillus Subtillus, and Bacillus simplex

271. The farm administration system of any one of embodiments 249-270, wherein the agricultural biological comprises a species of Azospirillum selected from A. brasilense, A. amazonense, A. irakense, A. lipoferum, A. largimobile, A. halopraeferens, A. oryzae, A. canadensis, A. doebereinerae, and A. melinis.

272. The farm administration system of any one of embodiments 249-271, wherein the agricultural biological comprises a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii

273. The farm administration system of any one of embodiments 249-272, wherein the agricultural biological is a seaweed extract from Reynoutria sachalinensis and/or Ascophyllum nodosum.

274. The farm administration system of any one of embodiments 249-273, wherein the biological is a biostimulant comprising a compound of Formula (2) or a salt, solvent, or isomer thereof, wherein Formula (2) comprises:

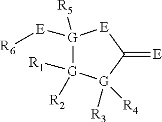

each E is independently O, S, or —NR₇;
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R⁸,

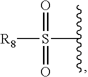

or a lone electron pair, wherein

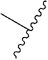

indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and
R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

275. The farm administration system of embodiment 274, wherein
each E is independently O,
each G is independently C,
R₁ and R₅, are each independently H,
R₂ and R₃ together form a bond
R₄ is substituted or unsubstituted alkyl, and
R₆ is substituted or unsubstituted heterocycloalkyl.

276. The farm administration system of any one of embodiments 249-275, wherein the biological is a biostimulant comprising a compound of Formula (3), wherein Formula (3) comprises:

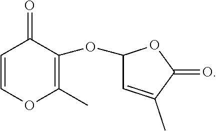 [SMILES: CC1=CC(Oc2c(C)occc2=O)OC1=O]

277. The farm administration system of any one of embodiments 249-276, wherein the biological is a biostimulant comprising a maltol compound.

278. The farm administration system of any one of embodiments 249-277, wherein the biological is a biostimulant comprising a lactone compound.

279. The farm administration system of any one of embodiments 249-278, wherein the biological is a biostimulant comprising a maltol lactone compound.

280. The farm administration system of any one of embodiments 249-279, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

281. The farm administration system of any one of embodiments 249-280, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743 and combinations thereof.

282. The farm administration system of any one of embodiments 249-281, wherein the engineered bacteria comprise at least one microbial species capable of fixing atmospheric nitrogen in the presence of exogenous nitrogen.

283. The farm administration system of any one of embodiments 249-282, wherein the engineered bacteria comprise an engineered diazotroph having increased nitrogen fixation activity as compared to an unmodified organism of the same species as said engineered, diazotroph, and wherein the genetic variation comprising genetic material that originates from at least one organism of the same species as said engineered diazotroph.

284. The farm administration system of any one of embodiments 249-283, wherein the engineered bacteria comprise an engineered, non-intergeneric diazotroph, wherein the genetic material of said engineered, non-intergeneric diazotroph consists essentially of genetic material that originates from at least one organism of the same species as said engineered, non-intergeneric diazotroph.

285. The farm administration system of any one of embodiments 249-284, wherein the engineered bacteria comprise an engineered diazotroph comprising at least one genetic variation introduced in a nitrogen fixation genetic regulatory network, whereby the engineered bacteria comprises increased expression or activity of nifH, increased expression or activity of nifA, and decreased expression or activity of NifL, wherein the at least one genetic variation comprises genetic material that originates from the same genus as said engineered diazotroph, whereby the engineered diazotroph has increased nitrogen fixation activity as compared to an unmodified organism of the same species as the engineered diazotroph.

286. The farm administration system of any one of embodiments 249-285, wherein the engineered bacteria does not comprise genetic material that originates from a different species than the remodeled bacteria.

287. The farm administration system of any one of embodiments 249-286, wherein the engineered bacteria comprise an epiphyte.

288. The farm administration system of any one of embodiments 249-287, wherein the engineered bacteria comprise an endophyte.

289. The farm administration system of any one of embodiments 249-288, wherein the engineered bacteria comprise a rhizophyte.

290. The farm administration system of any one of embodiments 249-289, wherein the engineered bacteria comprise at least one microbial species that is a non-intergeneric remodeled microbial species comprising an introduced control sequence operably linked to at least one gene of the nitrogen fixation or assimilation genetic regulatory network.

291. The farm administration system of any one of embodiments 249-290, wherein the engineered bacteria comprise at least one microbial species that is a non-intergeneric remodeled microbial species comprising a heterologous promoter operably linked to at least one gene of the nitrogen fixation or assimilation genetic regulatory network.

292. The farm administration system of any one of embodiments 249-291, wherein the engineered bacteria comprise at least one microbial species that is a non-intergeneric remodeled microbial species having at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD.

293. The farm administration system of any one of embodiments 249-292, wherein the engineered bacteria comprise at least one microbial species that is a non-intergeneric remodeled microbial species having a mutated nifL gene that has been altered to comprise a heterologous promoter inserted into said nif gene.

294. The farm administration system of any one of embodiments 249-293, wherein the engineered bacteria comprise at least one microbial species that is a non-intergeneric remodeled microbial species comprising a mutated glnE gene that results in a truncated GlnE protein lacking an adenylyl-removing (AR) domain.

295. The farm administration system of any one of embodiments 249-294, wherein the engineered bacteria comprise at least one microbial species that is a non-intergeneric remodeled microbial species comprising a mutated amtB gene that results in the lack of expression of said amtB gene.

296. The farm administration system of any one of embodiments 249-295, wherein the engineered bacteria comprise at least one microbial species that is a non-intergeneric remodeled microbial species comprising at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof.

297. The farm administration system of any one of embodiments 249-296, wherein the engineered bacteria comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, or combinations thereof.

298. The farm administration system of any one of embodiments 249-297, wherein the engineered bacteria comprise Kosakonia sacchari.

299. The farm administration system of any one of embodiments 249-298, wherein the engineered bacteria comprise Kosakonia sacchari PTA-126743.

300. The farm administration system of any one of embodiments 249-299, wherein the engineered bacteria comprise Klebsiella variicola.

301. The farm administration system of any one of embodiments 249-300, wherein the engineered bacteria comprise Klebsiella variicola PTA-126740.

302. The farm administration system of any one of embodiments 249-301, wherein the engineered bacteria comprise Klebsiella variicola PTA-126740 and Kosakonia sacchari PTA-126743.

303. The farm administration system of any one of embodiments 249-302, wherein the engineered bacteria are a liquid formulation.

304. The farm administration system of any one of embodiments 249-303, wherein the plurality of engineered bacteria are reconstituted from a previous powder formulation.

305. The farm administration system of any one of embodiments 249-304, wherein the engineered bacteria are at a concentration of between about 1.0×10⁴ and about 1.0×10¹² CFU/mL of the total volume of the composition.

306. The farm administration system of any one of embodiments 249-305, wherein the engineered bacteria are a powder formulation of lyophilized microbes.

307. The farm administration system of any one of embodiments 249-306, wherein the engineered bacteria and/or the agricultural biological are encapsulated within a water-soluble package.

308. The farm administration system of any one of embodiments 249-307, wherein the instructions comprise a recommended dose of the engineered bacteria and the agricultural biological to treat a plant species of Hordeum, Oryza, Zea, Sorghum, Brassica, or Triticeae.

309. The farm administration system of any one of embodiments 249-307, wherein the plant is corn.

310. The farm administration system of any one of embodiments 249-307, wherein the plant is soybean.

311. The farm administration system of any one of embodiments 249-307, wherein the plant is rice.

312. The farm administration system of any one of embodiments 249-307, wherein the plant is wheat.

313. The farm administration system of any one of embodiments 249-307, wherein the plant is rapeseed.

314. The farm administration system of any one of embodiments 249-307, wherein the plant is sweet corn, flint corn, popcorn, dent corn, pod corn, or flour corn.

315. The farm administration system of any one of embodiments 249-314, wherein the instructions comprise recommended application methods.

316. The farm administration system of embodiment 315, wherein the recommended application methods are selected from in-furrow, seed treatment, seedling root dip, broadcast, and foliar spray.

317. The farm administration system of any one of embodiments 249-316, wherein the instructions comprise a recommended order of applying the engineered bacteria and the agricultural biological.

1. A composition, comprising:
a) a plurality of engineered bacteria having at least one genetic variation introduced into at least one gene, or non-coding polynucleotide, of the nitrogen fixation or assimilation genetic regulatory network; and
b) an agricultural biological.

2. The composition of claim 1, wherein the plurality of engineered bacteria are non-intergeneric remodeled bacteria.

3.-7. (canceled)

8. The composition of claim 1, wherein the agricultural biological is:
a biostimulant that comprises humic substances, hormones, cell signaling molecules, seaweed extract, and/or amino acids,
a biochemical pesticide, a microbial pesticide, and a plant-incorporated-protectants pesticide, or
a biofertilizer selected from the group consisting of a bacterial, algal, and fungal biofertilizer.

9.-11. (canceled)

12. The composition of claim 1, wherein the agricultural biological comprises one or more species of cultured microbe selected from Methylobacterium, a mycorrhizal fungus, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

13. The composition of claim 1, wherein the agricultural biological is a mycorrhizal fungi selected from Glomus intraradices, Glomus mosseae, Glomus aggregatum, Glomus etunicatum, Glomus clarus, and Rhizophagus intraradices.

14. The composition of claim 1, wherein the agricultural biological is:
a species of Methylobacterium selected from M. gregans, M. adhaesivum, M. aerolatum, M. ajmalii, M. aquaticum aquaticum, M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile,
a species of Gluconacetobacter selected from G. azotocaptans, G. diazotrophicus, G. johannae, and G. sacchari,
a species of Bacillus selected from B. subtilis, B. simplex, B. methylotrophicus, B. amyloliquefaciens, B. megaterium, and B. licheniformis,
a species of Azospirillum selected from A. brasilense, A. amazonense, A. irakense, A. lipoferum, A. largimobile, A. halopraeferens, A. oryzae, A. canadensis, A. doebereinerae, and A. melinis, or
a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii.

15. (canceled)

16. The composition of claim 1, wherein the agricultural biological comprises
Azotobacter vinelandii and Clostridium pasteurianum or
a mycorrhizal fungus, Beauveria bassiana, Azospirillum sp., Azotobacter sp., and Rhodopseudomonas palustris or
a mycorrhizal fungus, Clostridium sp., and Azotobacter sp. or
Bacillus amyloliquefaciens and Trichoderma virens or
a species of Trichoderma selected from T. harzianum, T. atroviride, T. asperellum, and T. hamatum or
Ochrobactrum anthropic, Bacillus subtillus, and Bacillus simplex or
a seaweed extract from Reynoutria sachalinensis and/or Ascophyllum nodosum.

17.-25. (canceled)

26. The composition of claim 1, wherein the agricultural biological is a biostimulant comprising a compound of Formula (2) or a salt, solvent, or isomer thereof, wherein Formula (2) comprises: 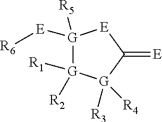
each E is independently O, S, or —NR₇;
each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈, 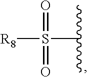
or a lone electron pair, wherein 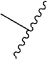
indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and
R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

27. The composition of claim 26, wherein
each E is independently O, each G is independently C,
R₁ and R₅, are each independently H, R₂ and R₃ together form a bond,
R₄ is substituted or unsubstituted alkyl, and
R₆ is substituted or unsubstituted heterocycloalkyl.

28. The composition of claim 1, wherein the agricultural biological is a biostimulant comprising a compound of Formula (3), wherein Formula (3) comprises:

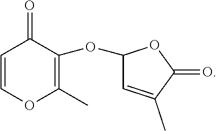 [SMILES: CC1=CC(Oc2c(C)occc2=O)OC1=O]

29.-31. (canceled)

32. The composition of claim 1, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

33. The composition of claim 1, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

34.-43. (canceled)

44. The composition of claim 1, wherein the engineered bacteria comprise at least one microbial species that is a non-intergeneric remodeled microbial species comprising:
at least one genetic variation introduced into at least one gene, or non-coding polynucleotide, of the nitrogen fixation or assimilation genetic regulatory network that results in one or more of: increased expression or activity of NifA or glutaminase; decreased expression or activity of NifL, NtrB, glutamine synthetase, GlnB, GlnK, DraT, AmtB; decreased adenylyl-removing activity of GlnE; or decreased uridylyl-removing activity of GlnD;
at least one of: a mutated nifL gene that has been altered to comprise a heterologous promoter inserted into said nifL gene; a mutated glnE gene that results in a truncated GlnE protein lacking an adenylyl-removing (AR) domain; a mutated amtB gene that results in the lack of expression of said amtB gene; a mutated glnD gene that results in a truncated GlnD protein lacking a uridyl-transferase domain or lack of expression of said glnD gene, and combinations thereof; or
at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifO, a gene associated with biosynthesis of a nitrogenase enzyme, bcsii, bcsiii, yjbE, fhaB, pehA, otsB, treZ, glsA2, and combinations thereof.

45.-51. (canceled)

52. The composition of claim 1, wherein the engineered bacteria comprises one or more of Enterobacter sacchari, Klebsiella variicola, Kosakonia sacchari, and Rahnella aquatilis.

53. (canceled)

54. The composition of claim 1, wherein the engineered bacteria comprise Klebsiella variicola PTA-126740 and Kosakonia sacchari PTA-126743.

55.-67. (canceled)

68. A method of treating a plant, comprising: applying the composition of claim 1 to a plant or plant part or to an area in which a plant will be grown or is growing.

69. (canceled)

70. The method of claim 68, wherein the plant is a species of Hordeum, Oryza, Zea, Sorghum, Brassica, or Triticeae.

71. The method of claim 68, wherein the plant is corn, soybean, rice, wheat, rapeseed, or any combination thereof.

72.-87. (canceled)

88. A method of treating a plant, comprising:
applying a plurality of engineered bacteria having at least one genetic variation introduced into at least one gene, or non-coding polynucleotide, of the nitrogen fixation or assimilation genetic regulatory network to a plant or to an area in which a plant will be grown or is growing; and
applying an agricultural biological to the plant or to an area in which a plant will be grown or is growing.

89. The method of claim 88, wherein the plurality of engineered bacteria are non-intergeneric remodeled bacteria.

90.-94. (canceled)

95. The method of claim 88, wherein the agricultural biological is:
a biostimulant that comprises humic substances, hormones, cell signaling molecules, seaweed extract, and/or amino acids,
a biochemical pesticide, a microbial pesticide, and a plant-incorporated-protectants pesticide, or
a biofertilizer selected from the group consisting of a bacterial, algal, and fungal biofertilizer.

96.-98. (canceled)

99. The method of claim 88, wherein the agricultural biological comprises one or more species of cultured microbe selected from Methylobacterium, a mycorrhizal fungus, Gluconacetobacter, Achromobacter, Agrobacterium, Anabaena, Azorhizobium, Azospirillum, Azotobacter, Bacillus, Beauveria, Bradyrhizobium, Clostridium, Enterobacter, Klebsiella, Kluyvera, Kosakonia, Mesorhizobium, Microbacterium, Ochrobactrum, Penicillium, Pseudomonas, Rahnella, Rhizobium, Rhodopseudomonas Sinorhizobium, Trichoderma, and combinations thereof.

100. The method of claim 88, wherein the agricultural biological is a mycorrhizal fungi selected from Glomus intraradices, Glomus mosseae, Glomus aggregatum, Glomus etunicatum, Glomus clarus, and Rhizophagus intraradices.

101. The method of claim 88, wherein the agricultural biological is:
a species of Methylobacterium selected from M. gregans, M. adhaesivum, M. aerolatum, M. ajmalii, M. aquaticum, M. brachiatum, M. brachythecii, M. bullatum, M. cerastii, M. crusticola, M. currus, M. dankookense, M. durans, M. frigidaeris, M. fujisawaense, M. funariae, M. gnaphalii, M. goesingense, M. gossipiicola, M. haplocladii, M. hispanicum, M. indicum, M. iners, M. isbiliense, M. jeotgali, M. komagatae, M. longum, M. marchantiae, M. mesophilicum, M. nodulans, M. nonmethylotrophicum, M. organophilum, M. oryzae, M. oryzihabitans, M. oxalidis, M. persicinum, M. phyllosphaerae, M. phyllostachyos, M. planium, M. platani, M. pseudosasicola, M. radiotolerans corrig., M. segetis, M. soli, M. symbioticum, M. tardum, M. tarhaniae, M. terrae, M. terricola, M. thuringiense, M. trifolii, and M. variabile variabile,
a species of Gluconacetobacter selected from G. azotocaptans, G. diazotrophicus, G. johannae, and G. sacchari,
a species of Bacillus selected from B. subtilis, B. simplex, B. methylotrophicus, B. amyloliquefaciens, B. megaterium, and B. licheniformis,
a species of Trichoderma selected from T. harzianum, T. atroviride, T. asperellum, and T. hamatum,
a species of Azospirillum selected from A. brasilense, A. amazonense, A. irakense, A. lipoferum, A. largimobile, A. halopraeferens, A. oryzae, A. canadensis, A. doebereinerae, and A. melinis, or
a species of Penicillium selected from P. bilaiae. P. brevicompactum, P. brocae, P. cecidicola, P. citrinum, P. coffeae, P. commune, P. crustosum, P. funiculosum, P. janthinellum, P. olsonii, P. oxalicum, P. radicum, P. ruqueforti, P. sclerotiorum, P. simplicissimum, and P. steckii.

102. (canceled)

103. The method of claim 88, wherein the agricultural biological comprises
Azotobacter vinelandii and Clostridium pasteurianum or
a mycorrhizal fungus, Beauveria bassiana, Azospirillum sp., Azotobacter sp., and Rhodopseudomonas palustris or
a mycorrhizal fungus, Clostridium sp., and Azotobacter sp. or
Bacillus amyloliquefaciens and Trichoderma virens or
Ochrobactrum anthropic, Bacillus subtillus, and Bacillus simplex or
a seaweed extract from Reynoutria sachalinensis and/or Ascophyllum nodosum.

104.-112. (canceled)

113. The method of claim 88, wherein the agricultural biological is a biostimulant comprising a compound of Formula (2) or a salt, solvent, or isomer thereof, wherein Formula (2) comprises: 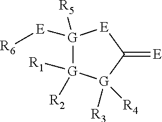
each E is independently O, S, or —NR₇; each G is independently C or N;
R₁, R₄, R₅, and R₆ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, substituted or unsubstituted heterocycloalkyl, —OR₈, —C(O)R₈, 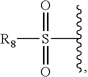
or a lone electron pair, wherein 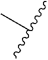
indicates a single bond;
R₂ and R₃ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl, or a lone electron pair; or R₂ and R₃ together form a bond, or form a substituted or unsubstituted aryl; and
R₇ and R₈ are each independently H, amino, halo, substituted or unsubstituted alkyl, substituted or unsubstituted aryl, substituted or unsubstituted heteroalkyl, substituted or unsubstituted arylalkyl, substituted or unsubstituted heteroaryl, substituted or unsubstituted heteroarylalkyl, substituted or unsubstituted cycloalkyl, or substituted or unsubstituted heterocycloalkyl.

114. The method of claim 113, wherein
each E is independently O,
each G is independently C,
R₁ and R₅, are each independently H,
R₂ and R₃ together form a bond,
R₄ is substituted or unsubstituted alkyl, and
R₆ is substituted or unsubstituted heterocycloalkyl.

115. The method of claim 88, wherein the agricultural biological is a biostimulant comprising a compound of Formula (3), wherein Formula (3) comprises:

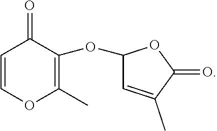 [SMILES: CC1=CC(Oc2c(C)occc2=O)OC1=O]

116.-118. (canceled)

119. The method of claim 88, wherein the plurality of engineered bacteria are selected from a species of the following genera: Achromobacter, Agrobacterium, Anabaena, Azorhizobium, Azospirillum, Azotobacter, Bacillus, Bradyrhizobium, Clostridium, Enterobacter, Klebsiella, Kluyvera, Kosakonia, Mesorhizobium, Microbacterium, Pseudomonas, Rahnella, Rhizobium, Sinorhizobium, and combinations thereof.

120. The method of claim 88, wherein the plurality of engineered bacteria comprise bacteria selected from: bacteria deposited as NCMA 201701002, bacteria deposited as NCMA 201708004, bacteria deposited as NCMA 201708003, bacteria deposited as NCMA 201708002, bacteria deposited as NCMA 201712001, bacteria deposited as NCMA 201712002, bacteria deposited as PTA-126575, bacteria deposited as PTA-126576, bacteria deposited as PTA-126577, bacteria deposited as PTA-126578, bacteria deposited as PTA-126579, bacteria deposited as PTA-126580, bacteria deposited as PTA-126584, bacteria deposited as PTA-126586, bacteria deposited as PTA-126587, bacteria deposited as PTA-126588, bacteria deposited as PTA-126740, bacteria deposited as PTA-126743, and combinations thereof.

121.-135. (canceled)

136. The method of claim 88, wherein the engineered bacteria comprises at least one genetic variation introduced into a member selected from the group consisting of: nifA, nifL, ntrB, ntrC, polynucleotide encoding glutamine synthetase, glnA, glnB, glnK, drat, amtB, polynucleotide encoding glutaminase, glnD, glnE, nifJ, nifH, nifD, nifK, nifY, nifE, nifN, nifU, nifS, nifV, nifW, nifZ, nifM, nifF, nifB, nifQ, a gene associated with biosynthesis of a nitrogenase enzyme, bcsii, bcsiii, yjbE, fhaB, pehA, otsB, treZ, glsA2, and combinations thereof.

137. (canceled)

138. (canceled)

139. The method of claim 88, wherein the engineered bacteria comprises one or more of Enterobacter sacchari, Klebsiella variicola, Kosakonia sacchari, and Rahnella aquatilis.

140. (canceled)

141. The method of claim 88, wherein the engineered bacteria comprise Klebsiella variicola PTA-126740 and Kosakonia sacchari PTA-126743.

142.-317. (canceled)
